(19) United States
(12) Patent Application Publication
(10) Pub. No.: US 2004/0110226 A1
(43) Pub. Date: Jun. 10, 2004

(54) Antibody optimization

(75) Inventors: Gregory Alan Lazar, Glendale, CA (US); John Rudolf Desjarlais, Pasadena, CA (US); Shannon Alicia Marshall, Pasadena, CA (US); Bassil I. Dahiyat, Altadena, CA (US)

Correspondence Address: Robin M. Silva, Dorsey & Whitney LLP, Intellectual Property Department, Four embarcadero Center, Suite 300, San Francisco, CA, 94111-4187, US

(73) Assignee: Xencor

(21) Appl. No.: 10/379,392

(22) Filed: Mar. 3, 2003

Related U.S. Application Data
(60) Provisional application No. 60/360,843, filed on Mar. 1, 2002.
(60) Provisional application No. 60/384,197, filed on May 29, 2002.

(51) Int. Cl. G01N 33/53; G06F 19/00; G01N 33/48; G01N 33/50

(52) U.S. Cl. 435/7.1; 702/19

(57) ABSTRACT

The present invention relates to the use of computational screening methods to optimize the physico-chemical properties of antibodies, including stability, solubility, and antigen binding affinity.

This application claims the benefit of the filing date of Ser. No. 60/360,843, filed Mar. 1, 2002 and Ser. No. 60/384,197, filed May 29, 2002, both of which are expressly incorporated by reference in their entirety.

### FIELD OF THE INVENTION

The present invention relates to the use of computational screening methods to optimize the physico-chemical properties of antibodies, including stability, solubility, and antigen binding affinity.

### BACKGROUND OF THE INVENTION

Monoclonal antibodies are in widespread use as therapeutics, diagnostics, and research reagents. As therapeutics, antibodies are used to treat a variety of conditions including cancer, autoimmune diseases, and cardiovascular disease. There are currently over ten approved antibody products on the US market, with over a hundred in development. Despite such acceptance and promise, there remains significant need for optimization of the structural and functional properties of antibodies.

The physical and chemical properties of antibody therapeutics significantly determine their performance during development, manufacturing, and clinical use. Antibodies may suffer from the stability and solubility issues similar to all proteins. Since fully developed antibody therapeutics require high levels of stability and solubility in order to retain activity through purification, formulation, storage, and administration, there is a need for effective methods to optimize antibody properties. Antibodies may be exposed to a variety of stresses, for example changes in temperature or pH, that may cause protein unfolding, destroy activity, or make the protein sensitive to proteolytic degradation. Proteins may be reengineered such that structure and activity are substantially more robust with respect to such stresses, for example, by optimizing intramolecular and interdomain interactions and by altering protease recognition sites.

Solubility is also of critical importance to antibody efficacy. Antibodies are typically formulated and administered at high concentration, conditions under which antibodies may form aggregates. Aggregates typically have poor activity and bioavailability, and are associated with increased immunogenicity. Solubility may also dictate which routes of administration are feasible. In many cases, antibody therapeutics have been limited to intravenous administration, because the antibody is not sufficiently soluble to allow formulation of an effective dose in the small volumes that are used for alternate routes of administration. In most cases, solubility obstacles have been considered as formulation problems that may be surmounted with exhaustive protein chemistry effort. However, such methods are inefficient, inconsistent, and time-consuming, often failing to yield soluble protein even following a significant expenditure of resources. Engineering approaches are beginning to emerge for the generation of soluble proteins; for example, in some cases solubility may be improved by replacing solvent exposed nonpolar residues with structurally compatible polar residues.

Another property of antibodies that frequently demands optimization is antigen-binding affinity. The binding affinity of an antibody for its biological target is a critical parameter for therapeutic efficacy. One particular case in which higher affinity is often sought is following humanization, herein defined as the reengineering of nonhuman antibodies to be more human-like in sequence. Humanization is carried out to reduce the immunogenicity of antibody therapeutics, but often results in loss of binding affinity for antigen. Regaining this affinity is typically desired during drug development. The main approach for enhancement of antigen affinity, herein referred to as affinity maturation, involves the engineering of mutations at positions that either directly contact antigen or indirectly influence binding. The demand for increased affinity for antigen is not, however, limited to humanization. Affinity maturation is frequently desired for therapeutic antibodies in general, whether they are derived from human, humanized, chimeric, or nonhuman sources.

Strategies for antibody optimization are sometimes carried out using random mutagenesis. In these cases positions are chosen randomly, or amino acid changes are made using simplistic rules. For example all residues may be mutated to alanine, referred to as alanine scanning. This can be used, for example, to map the antigen binding residues of an antibody (Kelley et al., 1993, Biochemistry 32:6828-6835; Vajdos et al., 2002, J. Mol. Biol. 320:415-428). The high level of sequence and structural similarity and large amount of sequence and structural information enable sequence-based methods of optimization. For example, sequence analysis has allowed significant characterization of the determinants of antibody stability and solubility (Ewert et al., 2003, J. Mol. Biol. 325:531-553; Ewert et al., 2003, Biochemistry 42:1517-1528), and can enable sequence-based methods of affinity maturation (see, U.S. Pat. No. 2003/0,022,240A1 and U.S. Pat. No. 2002/0,177,170A1, both hereby incorporated by reference). Sequence and structural information can be coupled with site-directed mutagenesis to engineer antibodies with enhanced biophysical properties (Worn & Plückthun, 2001, J. Mol. Biol. 305:989-1010; Wirtz & Steipe, 1999, Protein Sci. 8:2245-2250). More sophisticated engineering approaches for implementing antibody optimization strategies employ selection methods to screen higher levels of sequence diversity. As is well known in the art, there are a variety of selection technologies which may be used for such approaches, including, for example, display technologies such as phage display, ribosome display, yeast display, and the like. Selection methods coupled with random or rational mutagenesis have found utility for optimizing antibody stability (Jung et al., 1999, J. Mol. Biol. 294:163-180) and particularly for affinity maturation (Wu et al., 1999, J. Mol. Biol. 294:151-162; Schier et al., 1996, J. Mol. Biol. 255:28-43).

Despite some success, these current engineering strategies for antibody optimization suffer from three main obstacles. First, the level of sequence diversity that is wanted or needed can dramatically exceed that which is accessible by these technologies. The number of possible protein sequences grows exponentially with the number of positions that are randomized. Practical considerations including experimental and physical constraints such as transformation efficiency, instrumentation limits, and the like can significantly limit library size. Even for methods capable of screening large combinatorial libraries, this presents an obstacle. For example, the upper limit of diversity accessible by phage display is approximately 10⁹, which limits mutations to 7 positions if a fully random (all 20 amino acids) library is used.

A second limitation of current antibody engineering efforts is that experimental screens used to assess the fitness of antibody variants are not efficient, and therefore engineering optimized antibodies can be time- and resource-intensive, with no guarantee of success. Nor do current experimental screens always have the capacity to be implemented as a selection. For example, antibody stability is not a property that is readily selected for using a display technology. Screening for more stable antibodies would require purifying individual variants and determining their thermodynamic stability using time consuming biophysical methods.

A final limitation of current antibody engineering efforts is that constraints on proteins are not distinct. Instead, the determinants of antibody stability, solubility, and affinity for antigen are overlapping and the interactions that contribute to these properties are related. Thus, affinity maturation of an antibody may result in decreased stability, and optimization of an antibody's solubility may cause a loss in affinity for its antigen. This issue has important ramifications for antibody engineering because current experimental antibody optimization methods are poorly suited for simultaneous optimization of multiple, related properties. Consequently, a large portion of the candidates in experimental libraries are unsuitable. For example, a large fraction of sequence space encodes unfolded, misfolded, incompletely folded, partially folded, or aggregated proteins. Even among sequences that are folded and active, many will be less active, less soluble, or less stable than the wild type protein. In effect, current antibody engineering efforts generate experimental libraries that are composed of a large amount of “wasted” sequence space. More significantly, the probability of finding a suitable sequence decreases dramatically as the number of properties that are considered increases. Thus, there is a need for computational screening methods to optimize the physico-chemical properties of antibodies, including stability, solubility, and antigen binding affinity.

### SUMMARY OF THE INVENTION

The present invention provides methods of computational screening that may be applied to enhance the stability of antibodies, the solubility of antibodies, and the affinity of antibodies for antigen.

More specifically, the present invention discloses a method for optimizing at least one physico-chemical property of an antibody, wherein the method is executed by a computer under the control of a program, and the computer including a memory for storing said program, said method comprising the steps of: a. receiving a template antibody structure; b. selecting at least one variable position which belongs to said template antibody structure; c. selecting at least one amino acid to be considered at said variable positions; d. analyzing the interaction of each of said amino acids at each variable position with at least part of the remainder of said antibody, including said amino acids at other variable positions; and e. identifying a set of at least one antibody sequence with at least one optimized physico-chemical property.

The method of the present invention also optionally includes generating a library from the set of at least one antibody sequence and experimentally screening the library.

Computational screening methods have demonstrated their utility and success for the optimization of a broad array of protein properties. Application of these methods to antibodies represents a significant improvement because there are well known and established engineering strategies that are uniquely suited to antibodies. Computational screening is a hypothesis-driven method for engineering proteins, and thus the validity of the employed design strategies are critical to success. The application of these established engineering strategies as computational screening design strategies is not necessarily straightforward. However, as will be provided in detail, a number of aspects and parameters of the computational screening method may be adjusted to enable implementation of established antibody engineering strategies. Because all antibodies share a common structural template and high sequence similarity, and because of the enormous amount of sequence and structural information available, successful design strategies for the use of computational screening to optimize antibody stability, solubility, and affinity for antigen are broadly applicable to the entire family of antibodies. Finally, antibodies are often comprised of multiple similar domains. As a result, computational screening methods are uniquely modular for antibodies, that is to say that optimizations can be applied in an additive manner to engineer antibodies with a breadth of simultaneously enhanced functional and biophysical properties in multiple structural regions.

Computational screening methods of the present invention overcome the limitations of current antibody engineering methods. These methods are capitalizing on enormous recent advances in understanding of protein structure and function, substantial increases in the availability of high-resolution structures, and dramatic improvements in computing power. These methods offer a mechanism to explore sequence combinations that extend far beyond natural diversity, up to 10⁵⁰ or more sequences. Computational screening also enables the exploration of combinatorial complexity in the absence of experimentally selectable function, and thus biophysical properties such as stability and solubility, which are difficult to screen or select for, may be rationally screened in silico. Finally, computational screening methods offer the ability to algorithmically couple multiple constraints for simultaneous optimization of several protein properties. Thus experimental libraries that are designed using computational screening are composed primarily of productive sequence space. Computational screening may enrich experimental libraries with quality diversity, whether such experimental libraries are small such that members may be screened individually, or they are large such that selection methods are required for screening. As a result, computational screening increases the chances of identifying antibodies that are broadly optimized for stability, solubility, and affinity for antigen.

An additional benefit of computational screening methodology is that it is hypothesis driven (dash here). Thus successful strategies may be reapplied to antibodies as a whole, saving discovery cost and time. This is particularly relevant for antibodies because all antibodies share a common structural template and high sequence similarity, and because of the enormous amount of sequence and structural information available.

It is an object of the present invention to provide design strategies for the application of computational screening methods to enhance the stability of antibodies, to enhance the solubility of antibodies, and to affinity mature antibodies. Said design strategies describe the theoretical and/or experimental basis for their use, how the choice of variable positions and amino acids considered at those positions are carried out for their implementation, and ways in which experimental and sequence information may be used.

It is a further object of the present invention to provide computational methods for the application of computational screening methods to enhance the stability of antibodies, to enhance the solubility of antibodies, and to affinity mature antibodies. These computational methods describe a broad array of scoring functions, optimization algorithms, and the like for implementing computer programs to optimize antibodies. The computational methods further describe ways by which computational output may be used to generate experimental libraries of variants for experimental validation.

It is another object of the present invention to provide experimental methods for the application of computational screening technology to enhance the stability of antibodies, to enhance the solubility of antibodies, and to affinity mature antibodies. The experimental methods describe a broad array of molecular biology, protein production, and screening techniques that may be used to experimentally validate antibody variants that have been optimized for improved properties using computational screening methods.

In accordance with the objects outlined above, the present invention provides computational screening methods to optimize antibodies.

### BRIEF DESCRIPTION OF THE DRAWINGS

FIG. 1. Antibody structure and function. Shown is a model of a full-length human IgG1 antibody, constructed by combining the structure of the Campath Fab fragment (pdb accession code 1CE1), with the structure of the human IgG1 Fc region (pdb accession code 1DN2). The antibody is a homodimer of heterodimers, made up of two light chains and two heavy chains. The Ig domains that comprise the antibody are labeled, and include V_{L} and C_{L} for the light chain, and V_{H}, Cgamma1 (Cγ1), Cgamma2 (Cγ2), and Cgamma3 (Cγ3) for the heavy chain. Antibody regions relevant to the discussion are also labeled, including the variable region (Fv), the Fab region, and the Fc region. The regions which bind molecules or proteins relevant to the present invention are indicated, including the antigen binding site in the variable region, and the Fc region which binds FcγRS, FcRn, C1q, and proteins A and G. Campath is a registered trademark in the US of Burroughs Wellcome.

FIGS. 2a and 2b. Human germ line sequences and aligned antibody sequences. The sequences which are known to encode the human heavy chain variable region (V_{H}) and the human kappa light chain variable region (V_{L}) are shown aligned with four relevant antibody sequences. The germ line sequences were obtained from the IMGT database (IMGT, the international ImMunoGeneTics information system®; imgt.cines.fr), and aligned and numbered according to the numbering scheme of Chothia (Chothia et a., 1992, J Mol. Biol. 227, 776-798, 799-817; Tomlinson et a., 1995, EMBO J. 14:4628-4638; Williams et a., 1996, J. Mol. Biol. 264:220-232; Al-Lazikani et al., 1997, J. Mol. Biol. 273, 927-948; Chothia et al., 1998, J. Mol. Biol. 278, 457-479; all of which are herein expressly incorporated by reference). The regions of the variable region are indicated above the numbering, and these include framework regions 1 through 3 (FR1, FR2, and FR3) and the complementarity determining regions (CDRs) 1 through 3 (CDR1, CDR2, and CDR3). As is known in the art, V_{H} CDR3 is not a part of the V_{H} germ line and V_{L} CDR3 is encoded only up to Chothia position 95 in the V_{L} kappa germ line. Positions that make up CDRs are underlined. The germ line chains are grouped into 7 subfamilies for both V_{H} and V_{L}, as is known in the art, and these subfamilies are grouped together and separated by a blank line. Four antibody sequences used in the examples of the present invention, listed by their pdb accession codes and underlined, are shown below the subfamily to which they are closest in sequence. These sequences were aligned using the alignment program BLAST. The most similar germ line sequences to these four antibodies, as determined by this alignment analysis, are shown in parentheses next to the antibody code. The most similar germ line V_{H} chains to the four antibodies are VH_{—}3-74 for D3H44 (1JPT), VH_{—}3-66 for Herceptin (1FVC), VH_{—}4-59 and VH_{—}3-72 for Campath (1CE1), and VH_{—}7-4-1 for rhumAb VEGF (1CZ8). The most similar germ line V_{L} chains to the four antibodies are VLk_{—}1D-3 for D3H44 (1JPT), VLk_{—}1D-3 for Herceptin (1FVC), VLk_{—}1D-33 for Campath (1CE1), and VLk_{—}1D-33 for rhumAb VEGF (1CZ8). Herceptin is a registered trademark in the US owned by Genentech, Inc.

FIG. 3. Antibody structures relevant to the presented examples. The seven antibody structures used in the present invention are listed. For each antibody is listed the target antigen, the source, the pdb accession code, whether the structure is a complex of the antibody with antigen (bound) or is uncomplexed (unbound), the resolution, and the reference.

FIG. 4. Campath V_{H} domain stabilization. The large central figure shows the Campath V_{H} domain from 1CE1 as a gray ribbon diagram, with Example 1 variable position residues represented as black lines. The smaller figure in the upper left shows the modeled full-length antibody structure (from FIG. 1) with the relevant domain highlighted by a box.

FIGS. 5a, 5b, and 5c. Campath V_{H} domain stabilization. FIG. 5a shows the results of the computational screening calculations described in Example 1. Column 1 lists the heavy (H) chain variable positions. Column 2 lists the amino acids considered at each variable position. The set of amino acids belonging to the Core classification are described in the section entitled “Selection of Amino Acids to be Considered at Each Position”. Column 3 lists the WT Campath amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIGS. 5b and 5c show experimental libraries derived from the computational screening results, as described in Example 1. Column 1 lists variable positions and column 2 shows amino acid substitutions that are included in the experimental library. FIG. 5c is represented combinatorially, that is the explicit library is the combination of each possible amino acid substitution at each variable position with all other possible amino acid substitutions at all other positions. The complexity of the library, that is the total number of defined sequences of which it is composed, is shown in the bottom row.

FIG. 6. Campath V_{L} domain stabilization. The large central figure shows the Campath V_{L} domain from 1CE1 as a gray ribbon diagram, with Example 2 variable position residues represented as black lines. The smaller figure in the upper left shows the modeled full-length antibody structure with the relevant domain highlighted by a box.

FIGS. 7a and 7b. Campath V_{L} domain stabilization. FIG. 7a shows the results of the computational screening calculations described in Example 2. Column 1 lists the light (L) chain variable positions. Column 2 lists the amino acids considered at each variable position. The set of amino acids belonging to the Core and Boundary classifications are described in the section entitled “Selection of Amino Acids to be Considered at Each Position”. Column 3 lists the WT Campath amino acid identity at each variable position. Column 4 lists the amino acid identity at each variable position in the DEE ground state sequence predicted by the computational screening calculations. Column 5 lists the set of amino acids at each variable position which are observed in the Monte Carlo output. Each amino acid is followed by its occupancy, that is the number of sequences in the 1000 sequence set that contain that amino acid at that variable position. FIG. 7b shows an experimental library derived from the computational screening results, as described in Example 2. Column 1 lists variable positions and column 2 shows amino acid substitutions which are included in the experimental library. The library is represented combinatorially, that is the explicit library is the combination of each possible amino acid substitution at each variable position with all other possible amino acid substitutions at all other positions. The complexity of the library, that is the total number of defined sequences of which it is composed, is shown in the bottom row.

FIG. 8. Campath V_{H} Cγ1 domain stabilization. The large central figure shows the Campath V_{H} Cγ1 domain from 1CE1 as a gray ribbon diagram, with Example 3 variable position residues represented as black lines. The smaller figure in the upper left shows the modeled full-length antibody structure with the relevant domain highlighted by a box.

FIGS. 9a and 9b. Campath V_{H} Cγ1 domain stabilization. FIG. 9a shows the results of the computational screening calculations described in Example 3. Column 1 lists the heavy (H) chain variable positions. Column 2 lists the amino acids considered at each variable position. The set of amino acids belonging to the Core and Boundary classifications are described in the section entitled “Selection of Amino Acids to be Considered at Each Position”. Column 3 lists the WT Campath amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIG. 9b shows an experimental library derived from the computational screening results, as described in Example 3. Column 1 lists variable positions, and column 2 shows amino acid substitutions that are included in the experimental library. The library is represented combinatorially, that is the explicit library is the combination of each possible amino acid substitution at each variable position with all other possible amino acid substitutions at all other positions. The complexity of the library, that is the total number of defined sequences of which it is composed, is shown in the bottom row.

FIG. 10. Fc V_{H} Cγ2 domain stabilization. The large central figure shows the Fc V_{H} Cγ2 domain from 1DN2 as a gray ribbon diagram, with Example 4 variable position residues represented as black lines. The smaller figure in the upper left shows the modeled full-length antibody structure with the relevant domain highlighted by a box.

FIGS. 11a and 11b. Fc V_{H} Cγ2 domain stabilization. FIG. 11a shows the results of the computational screening calculations described in Example 4. Column 1 lists the heavy (H) chain variable positions. Column 2 lists the amino acids considered at each variable position. The set of amino acids belonging to the Core and Boundary classifications are described in the section entitled “Selection of Amino Acids to be Considered at Each Position”. Column 3 lists the WT Campath amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIG. 11b shows an experimental library derived from the computational screening results, as described in Example 4. Column 1 lists variable positions, and column 2 shows amino acid substitutions that are included in the experimental library. The library is represented combinatorially, that is the explicit library is the combination of each possible amino acid substitution at each variable position with all other possible amino acid substitutions at all other positions. The complexity of the library, that is the total number of defined sequences of which it is composed, is shown in the bottom row.

FIG. 12. Fc V_{H} Cγ3 domain stabilization. The large central figure shows the Fc V_{H} Cγ3 domain from 1DN2 as a gray ribbon diagram, with Example 5 variable position residues represented as black lines. The smaller figure in the upper left shows the modeled full-length antibody structure with the relevant domain highlighted by a box.

FIGS. 13a and 13b. Fc V_{H} Cγ3 domain stabilization. FIG. 13a shows the results of the computational screening calculations described in Example 5. Column 1 lists the heavy chain variable positions. Column 2 lists the amino acids considered at each variable position. The set of amino acids belonging to the Core and Boundary classifications are described in the section entitled “Selection of Amino Acids to be Considered at Each Position”. Column 3 lists the WT Fc amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIG. 13b shows an experimental library derived from the computational screening results, as described in Example 5. Column 1 lists variable positions, and column 2 shows amino acid substitutions that are included in the experimental library. The library is represented combinatorially, that is the explicit library is the combination of each possible amino acid substitution at each variable position with all other possible amino acid substitutions at all other positions. The complexity of the library, that is the total number of defined sequences of which it is composed, is shown in the bottom row.

FIG. 14. rhumAb VEGF V_{H}/V_{L} interface stabilization. The large central figure shows the rhumAb VEGF V_{H} and V_{L} domains from 1CZ8 as black and gray ribbons respectively, with Example 6 variable position residues represented as black lines. The smaller figure in the upper left shows the modeled full-length antibody structure with the relevant region highlighted by a box.

FIGS. 15a, 15b, and 15c. rhumAb VEGF V_{H}/V_{L} interface stabilization. FIGS. 15a and 15b show the results of the computational screening calculations described in Example 6. Column 1 lists the light (L) and heavy (H) chain variable positions. Column 2 lists the amino acids considered at each variable position. The set of amino acids belonging to the Core and Boundary classifications are described in the section entitled “Selection of Amino Acids to be Considered at Each Position”. Column 3 lists the WT rhumAb VEGF amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIG. 15c shows an experimental library derived from the computational screening results, as described in Example 6. Column 1 lists variable positions, and column 2 shows amino acid substitutions that are included in the experimental library. The library is represented combinatorially, that is the explicit library is the combination of each possible amino acid substitution at each variable position with all other possible amino acid substitutions at all other positions. The complexity of the library, that is the total number of defined sequences of which it is composed, is shown in the bottom row.

FIGS. 16a and 16b. Sequence alignment of rhumAb VEGF variable region with the human variable region germ line. The rhumAb VEGF V_{H} and V_{L} sequences are shown aligned with the sequences that encode the human V_{H} (FIG. 16a) and V_{L} (FIG. 16b) germ line. The germ line sequences were obtained from the IMGT database, and numbered according to the numbering scheme of Chothia. The regions of the variable region are indicated above the numbering, and these include framework regions 1 through 3 (FR1, FR2, and FR3) and the complementarity determining regions (CDRs) 1 through 3 (CDR1, CDR2, and CDR3). Positions that make up CDRs are underlined. The 7 germ line subfamilies for V_{H} and V_{L} are grouped together and separated by a blank line. The rhumAb VEGF V_{H} and V_{L} sequences were aligned to the germ line sequences using the alignment program BLAST. rhumAb VEGF V_{H} is most similar to the germ line chain VH_{—}7-4-1, and rhumAb VEGF V_{L} is most similar to the germ line chain VLk_{—}1D-33. The rhumAb VEGF V_{H} and V_{L} sequences are indicated by the underlined pdb accession code 1CZ8, and shown below the subfamily to which they are closest in sequence. Amino acids at variable positions for Example 6 design calculations are shown in bold in the 1CZ8 and the germ line sequences.

FIGS. 17a and 17b. rhumAb VEGF sequence-guided V_{H}/V_{L} interface stabilization. FIG. 17a shows the results of the computational screening calculations described in Example 6. Rows 1 through 5 list the chain (L, light chain or H, heavy chain), variable positions as defined in the 1CZ8 structure and the according to the Chothia numbering scheme, amino acids considered at those positions as obtained from FIGS. 16a and 16b, and the amino acid at each position in the WT rhumAb VEGF sequence. “All” or “All 20” means that all 20 amino acids are considered at the variable position. The rows that follow list the amino acid identity at variable positions for the lowest energy sequence from each cluster group, as described in Example 6. FIG. 17a is similar to FIG. 17b except that all the listed sequences are the set of sequences make up cluster group 5.

FIG. 18. Herceptin V_{H}/V_{L} interface stabilization. The large central figure shows the Herceptin V_{H} and V_{L} domains from 1FVC as black and gray ribbons respectively, with Example 7 variable position residues represented as black lines. The smaller figure in the upper left shows the modeled full-length antibody structure with the relevant region highlighted by a box.

FIGS. 19a, 19b, 19c, and 19d. Herceptin V_{H}/V_{L} interface stabilization. FIGS. 19a and 19c show the results of the computational screening calculations described in Example 7. Column 1 lists the light (L) and heavy (H) chain variable positions. Column 2 lists the amino acids considered at each variable position. The set of amino acids belonging to the Core, Surface, and Boundary classifications are described in the section entitled “Selection of Amino Acids to be Considered at Each Position”. Column 3 lists the WT Herceptin amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIGS. 19b and 19d show experimental libraries derived from the computational screening results, as described in Example 7. Column 1 lists variable positions, and column 2 shows amino acid substitutions that are included in the experimental library. The libraries are represented combinatorially, that is the explicit library is the combination of each possible amino acid substitution at each variable position with all other possible amino acid substitutions at all other positions. The complexity of the libraries, that is the total number of defined sequences of which it is composed, is shown in the bottom row.

FIG. 20. rhumAb VEGF C_{L}/Cγ1 interface stabilization. The large central figure shows the VEGF C_{L} and Cγ1 domains from 1CZ8 as black and gray ribbons respectively, with Example 8 variable position residues represented as black lines. The smaller figure in the upper left shows the modeled full-length antibody structure with the relevant region highlighted by a box.

FIGS. 21a and 21b. rhumAb VEGF C_{L}/Cγ1 interface stabilization. FIG. 21a shows the results of the computational screening calculations described in Example 8. Column 1 lists the light (L) and heavy (H) chain variable positions. Column 2 lists the amino acids considered at each variable position. The set of amino acids belonging to the Core classifications are described in the section entitled “Selection of Amino Acids to be Considered at Each Position”. Column 3 lists the WT rhumAb VEGF amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIG. 21b shows an experimental library derived from the computational screening results, as described in Example 8. Column 1 lists variable positions, and column 2 shows amino acid substitutions that are included in the experimental library. The libraries are represented combinatorially, that is the explicit library is the combination of each possible amino acid substitution at each variable position with all other possible amino acid substitutions at all other positions. The complexity of the libraries, that is the total number of defined sequences of which it is composed, is shown in the bottom row.

FIG. 22. Fc Cγ3/Cγ3 interface stabilization. The large central figure shows the Fc Cγ3 domains from 1DN2 as gray ribbons, with Example 9 variable position residues represented as black lines. The smaller figure in the upper left shows the modeled full-length antibody structure with the relevant region highlighted by a box.

FIGS. 23a and 23b. Fc Cγ3/Cγ3 interface stabilization. FIG. 23a shows the results of the computational screening calculations described in Example 9. Column 1 lists the heavy chain variable positions. Chains A and B are the two symmetrical Cγ3 domains in the 1DN2 structure. Column 2 lists the amino acids considered at each variable position. The set of amino acids belonging to the Core classifications are described in the section entitled “Selection of Amino Acids to be Considered at Each Position”. Column 3 lists the WT Fc amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIG. 23b shows an experimental library derived from the computational screening results, as described in Example 9. Column 1 lists variable positions, and column 2 shows amino acid substitutions that are included in the experimental library. The libraries are represented combinatorially, that is the explicit library is the combination of each possible amino acid substitution at each variable position with all other possible amino acid substitutions at all other positions. The complexity of the libraries, that is the total number of defined sequences of which it is composed, is shown in the bottom row.

FIG. 24. Campath solubility optimization. The large central figure shows the Campath Fab fragment from 1CE1 as a gray ribbon diagram, with Example 10 variable position residues represented as black ball and sticks. The smaller figure in the upper left shows the modeled full-length antibody structure with the relevant region highlighted by a box.

FIGS. 25a and 25b. Campath solubility optimization. FIG. 25a shows the results of the computational screening calculations described in Example 10. Column 1 lists the heavy (H) and light (L) chain variable positions. Column 2 lists the wild type amino acid identity at each variable position. The remaining 20 columns indicate which of the 20 natural amino acids are favorable substitutions for each variable position according to the computational screening calculations. The presence of an amino acid in its column for a variable position indicates that the amino acid is within 1 unit of energy of the lowest energy substitution. FIG. 25b shows an experimental library derived from the computational screening results, as described in Example 10. Column 1 lists variable positions, and column 2 shows amino acid substitutions that are included in the experimental library. The library is represented combinatorially, i.e. the explicit library is the combination of each possible amino acid substitution at each variable position with all other possible amino acid substitutions at all other positions. The complexity of the library, that is the total number of defined sequences of which it is composed, is shown in the bottom row.

FIG. 26. rhumAb VEGF solubility optimization. The large central figure shows the rhumAb VEGF Fab fragment from 1CZ8 as a gray ribbon diagram, with Example 11 variable position residues represented as black ball and sticks. The smaller figure in the upper left shows the modeled full-length antibody structure with the relevant region highlighted by a box.

FIGS. 27a and 27b. rhumAb VEGF solubility optimization. FIG. 27a shows the results of the computational screening calculations described in Example 11. Column 1 lists the heavy (H) and light (L) chain variable positions. Column 2 lists the wild type amino acid identity at each variable position. The remaining 20 columns indicate which of the 20 natural amino acids are favorable substitutions for each variable position according to the computational screening calculations. The presence of an amino acid in its column for a variable position indicates that the amino acid is within 1 unit of energy of the lowest energy substitution. FIG. 27b shows an experimental library derived from the computational screening results, as described in Example 11. Column 1 lists variable positions, and column 2 shows amino acid substitutions that are included in the experimental library. The library is represented combinatorially, i.e. the explicit library is the combination of each possible amino acid substitution at each variable position with all other possible amino acid substitutions at all other positions. The complexity of the library, that is the total number of defined sequences of which it is composed, is shown in the bottom row.

FIG. 28. Herceptin solubility optimization. The large central figure shows the Herceptin scFv fragment from 1FVC as a gray ribbon diagram, with Example 12 variable position residues represented as black ball and sticks. The smaller figure in the upper left shows the modeled full-length antibody structure with the relevant region highlighted by a box.

FIGS. 29a and 29b. Herceptin solubility optimization. FIG. 29a shows the results of the computational screening calculations described in Example 12. Column 1 lists the heavy (H) and light (L) chain variable positions. Column 2 lists the wild type amino acid identity at each variable position. The remaining 20 columns indicate which of the 20 natural amino acids are favorable substitutions for each variable position according to the computational screening calculations. The presence of an amino acid in its column for a variable position indicates that the amino acid is within 1 unit of energy of the lowest energy substitution. FIG. 29b shows an experimental library derived from the computational screening results, as described in Example 12. Column 1 lists variable positions, and column 2 shows amino acid substitutions that are included in the experimental library. The library is represented combinatorially, i.e. the explicit library is the combination of each possible amino acid substitution at each variable position with all other possible amino acid substitutions at all other positions. The complexity of the library, that is the total number of defined sequences of which it is composed, is shown in the bottom row.

FIG. 30. Fc solubility optimization. The large central figure shows the Fc region from 1DN2 as a gray ribbon diagram, with Example 13 variable position residues represented as black ball and sticks. The smaller figure in the upper left shows the modeled full-length antibody structure with the relevant region highlighted by a box.

FIGS. 31a and 31b. Fc solubility optimization. FIG. 31a shows the results of the computational screening calculations described in Example 13. Column 1 lists the heavy chain variable positions for the A chain, i.e. for only one of the Cγ2-Cγ3 heavy chains of the homodimer. Column 2 lists the wild type amino acid identity at each variable position. The remaining 20 columns indicate which of the 20 natural amino acids are favorable substitutions for each variable position according to the computational screening calculations. The presence of an amino acid in its column for a variable position indicates that the amino acid is within 1 unit of energy of the lowest energy substitution. FIG. 31b shows an experimental library derived from the computational screening results, as described in Example 13. Column 1 lists variable positions, and column 2 shows amino acid substitutions that are included in the experimental library. The library is represented combinatorially, i.e. the explicit library is the combination of each possible amino acid substitution at each variable position with all other possible amino acid substitutions at all other positions. The complexity of the library, that is the total number of defined sequences of which it is composed, is shown in the bottom row.

FIG. 32. rhumAb VEGF affinity maturation. The large central figure shows the 1CZ8 rhumAb VEGF V_{H} and V_{L} domains as gray ribbons bound to the VEGF target antigen as black ribbon, with Example 14 variable position residues represented as black lines. The smaller figure in the upper left shows the modeled full-length antibody structure with the relevant region highlighted by a box.

FIGS. 33a and 33b. rhumAb VEGF affinity maturation. FIG. 33a shows the results of the computational screening calculations described in Example 14. Column 1 lists the light (L) and heavy (H) chain variable positions. Column 2 lists the amino acids considered at each variable position. The set of amino acids belonging to the Core, Surface, and Boundary classifications are described in the section entitled “Selection of Amino Acids to be Considered at Each Position”. Column 3 lists the WT rhumAb VEGF amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIG. 33b shows an experimental library derived from the computational screening results, as described in Example 14. Column 1 lists variable positions, and column 2 shows amino acid substitutions that are included in the experimental library. The libraries are represented combinatorially, that is the explicit library is the combination of each possible amino acid substitution at each variable position with all other possible amino acid substitutions at all other positions. The complexity of the libraries, that is the total number of defined sequences of which it is composed, is shown in the bottom row. FIG. 34. rhumAb VEGF affinity maturation. The large central figure shows the 1CZ8 rhumAb VEGF V_{H} and V_{L} domains as gray ribbons bound to the VEGF target antigen shown as black ribbon, with Example 14 variable position residues represented as black lines. The smaller figure in the upper left shows the modeled full-length antibody structure with the relevant region highlighted by a box.

FIGS. 35a and 35b. rhumAb VEGF affinity maturation. FIG. 35a shows the results of the computational screening calculations described in Example 14. Column 1 lists the light (L) and heavy (H) chain variable positions. Column 2 lists the amino acids considered at each variable position. The set of amino acids belonging to the Core, Surface, and Boundary classifications are described in the section entitled “Selection of Amino Acids to be Considered at Each Position”. Column 3 lists the WT rhumAb VEGF amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIG. 35b shows an experimental library derived from the computational screening results, as described in Example 14. Column 1 lists variable positions, and column 2 shows amino acid substitutions that are included in the experimental library. The libraries are represented combinatorially, that is the explicit library is the combination of each possible amino acid substitution at each variable position with all other possible amino acid substitutions at all other positions. The complexity of the libraries, that is the total number of defined sequences of which it is composed, is shown in the bottom row.

FIG. 36. SM3 affinity maturation. The large central figure shows the 1SM3 V_{H} and V_{L} domains as gray ribbons bound to the MUC1 antigen shown as black ribbon, with Example 15 variable position residues represented as black lines. The smaller figure in the upper left shows the modeled full-length antibody structure with the relevant region highlighted by a box.

FIGS. 37a, 37b, and 37c. SM3 affinity maturation. FIGS. 37a and 37b show the results of the computational screening calculations described in Example 15. Column 1 lists the light (L) and heavy (H) chain variable positions. Column 2 lists the amino acids considered at each variable position. The set of amino acids belonging to the Core, Surface, and Boundary classifications are described in the section entitled “Selection of Amino Acids to be Considered at Each Position”. Column 3 lists the WT SM3 amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIG. 37c shows an experimental library derived from the computational screening results, as described in Example 15. Column 1 lists variable positions, and column 2 shows amino acid substitutions that are included in the experimental library. The libraries are represented combinatorially, that is the explicit library is the combination of each possible amino acid substitution at each variable position with all other possible amino acid substitutions at all other positions. The complexity of the libraries, that is the total number of defined sequences of which it is composed, is shown in the bottom row.

FIG. 38. Campath affinity maturation. The large central figure shows the 1CE1 V_{H} and V_{L} domains as gray ribbons bound to the CD52 antigen shown as black ribbon, with Example 16 variable position residues represented as black lines. The smaller figure in the upper left shows the modeled full-length antibody structure with the relevant region highlighted by a box.

FIGS. 39a and 37b. Campath affinity maturation. FIG. 39a shows the results of the computational screening calculations described in Example 16. Column 1 lists the light (L) and heavy (H) chain variable positions. Column 2 lists the amino acids considered at each variable position. The set of amino acids belonging to the Core, Surface, and Boundary classifications are described in the section entitled “Selection of Amino Acids to be Considered at Each Position”. Column 3 lists the WT Campath amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIG. 39b shows an experimental library derived from the computational screening results, as described in Example 16. Column 1 lists variable positions, and column 2 shows amino acid substitutions that are included in the experimental library. The libraries are represented combinatorially, that is the explicit library is the combination of each possible amino acid substitution at each variable position with all other possible amino acid substitutions at all other positions. The complexity of the libraries, that is the total number of defined sequences of which it is composed, is shown in the bottom row.

FIGS. 40a and 40b. Sequence alignment of Campath variable region with the human variable region germ line. The Campath V_{H} and V_{L} sequences are shown aligned with the sequences that encode the human V_{H} (FIG. 40a) and V_{L} (FIG. 40b) germ line. The germ line sequences were obtained from the IMGT database, and numbered according to the numbering scheme of Chothia. The regions of the variable region are indicated above the numbering, and these include framework regions 1 through 3 (FR1, FR2, and FR3) and the complementarity determining regions (CDRs) 1 through 3 (CDR1, CDR2, and CDR3). Positions that make up CDRs are underlined. The 7 germ line subfamilies for V_{H} and V_{L} are grouped together and separated by a blank line. The Campath V_{H} and V_{L} sequences were aligned to the germ line sequences using the alignment program BLAST. Campath V_{H} is most similar to the germ line chain VH_{—}4-59 and VH_{—}3-72, and Campath V_{L} is most similar to the germ line chain VLk_{—}1D-33. The Campath V_{H} and V_{L} sequences are indicated by the underlined pdb accession code 1CE1, and shown below the subfamily to which they are closest in sequence. Amino acids at variable positions for Example 16 design calculations are shown in bold in the 1CE1 and the germ line sequences.

FIGS. 41a and 41b. Campath sequence-guided affinity maturation. FIG. 41a shows the results of the computational screening calculations described in Example 16. Rows 1 through 3 list the light (L) or heavy (H) chain variable positions, as defined in the 1CE1 structure, and the according to the Chothia numbering scheme. Row 4 lists the amino acids considered at variable positions as obtained from FIGS. 40a and 40b, and row 5 lists the amino acid at each position in the WT Campath sequence. “All” or “All 20” means that all 20 amino acids are considered at the variable position. The rows that follow list the amino acid identity at variable positions for the lowest energy sequence from each cluster group, as described in Example 16. FIG. 41b is similar to FIG. 41a except that all the listed sequences are the set of sequences make up cluster groups 4 and 9.

FIG. 42. D3H44 affinity maturation. The large central figure shows the 1JPS V_{H} and V_{L} domains as gray ribbons bound to the tissue factor antigen shown as black ribbon, with Example 16 variable position residues represented as black lines. The smaller figure in the upper left shows the modeled full-length antibody structure with the relevant region highlighted by a box.

FIGS. 43a, 43b, 43c, and 43d. D3H44 affinity maturation. FIGS. 43a and 43b show the results of the computational screening calculations using the 1JPS template and 1JPT template respectively, as described in Example 17. Column 1 lists the light (L) and heavy (H) chain variable positions. Column 2 lists the amino acids considered at each variable position. The set of amino acids belonging to the Core, Surface, and Boundary classifications are described in the section entitled “Selection of Amino Acids to be Considered at Each Position”. Column 3 lists the WT D3H44 amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIGS. 43c and 43d show an experimental library derived from the computational screening results, as described in Example 17. In FIG. 43c, column 1 lists variable positions, and columns 2 and 3 show amino acid substitutions, which are included in the experimental library. In FIG. 43d, column 1 lists variable positions, and column 2 shows amino acid substitutions that are included in the experimental library. The libraries are represented combinatorially, that is the explicit library is the combination of each possible amino acid substitution at each variable position with all other possible amino acid substitutions at all other positions. The complexity of the libraries, that is the total number of defined sequences of which it is composed, is shown in the bottom row.

FIG. 44. Herceptin affinity maturation. The large central figure shows the 1FVC V_{H} and V_{L} domains as black and gray ribbons respectively, with Example 18 variable position residues represented as black lines. The smaller figure in the upper left shows the modeled full-length antibody structure with the relevant region highlighted by a box.

FIGS. 45a and 45b. Herceptin affinity maturation. FIG. 45a shows the results of the computational screening calculations described in Example 18. Column 1 lists the light (L) and heavy (H) chain variable positions. Column 2 lists the amino acids considered at each variable position. The set of amino acids belonging to the Core, Surface, and Boundary classifications are described in the section entitled “Selection of Amino Acids to be Considered at Each Position”. Column 3 lists the WT Herceptin amino acid identity at each variable position. Column 4 lists the amino acid identity at each variable position in the DEE ground state sequence predicted by the computational screening calculations. Column 5 lists the set of amino acids at each variable position that are observed in the Monte Carlo output. Each amino acid is followed by its occupancy, that is the number of sequences in the 1000 sequence set that contain that amino acid at that variable position. FIG. 45b shows an experimental library derived from the computational screening results, as described in Example 18. Column 1 lists variable positions, and column 2 shows amino acid substitutions that are included in the experimental library. The libraries are represented combinatorially, that is the explicit library is the combination of each possible amino acid substitution at each variable position with all other possible amino acid substitutions at all other positions. The complexity of the libraries, that is the total number of defined sequences of which it is composed, is shown in the bottom row.

### DETAILED DESCRIPTION OF THE INVENTION

The present invention is directed to the use of a variety of computational methods to alter physico-chemical properties of antibodies, to allow the virtual screening of large numbers of potential variants to arrive at sets that exhibit desirable properties as compared to the starting antibody or antibodies. The computational analyses can be done as a single step, with the resulting set being experimentally generated and tested in the desired assay, for improved function and properties. Similarly, the original set can be additionally computationally manipulated to create a new library which then itself can be experimentally tested.

The invention finds use in the prescreening of variant antibody libraries; that is, computational screening for stability (or other properties) may be done on either the entire protein or some subset of residues, as desired and described below. By using computational methods to generate a threshold or cutoff to eliminate disfavored sequences, the percentage of useful variants in a given variant set size can increase, and the required experimental outlay is decreased.

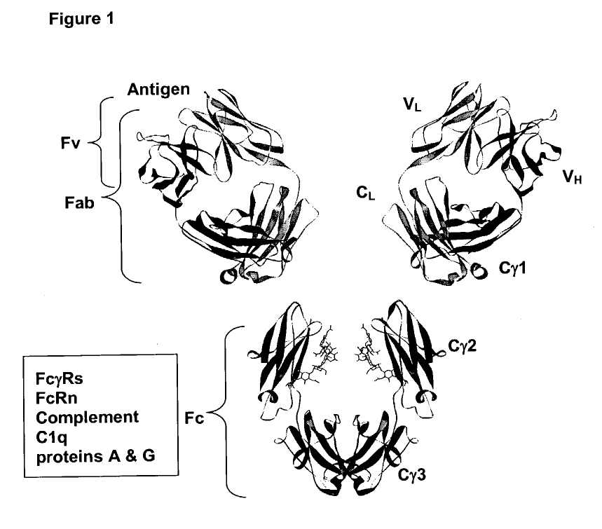

In order that the invention may be more completely understood, several definitions are set forth below. By “affinity maturation” herein is meant the process of enhancing the affinity of an antibody for its antigen. Methods for affinity maturation include but are not limited to computational screening methods and experimental methods. By “antibody” herein is meant a protein consisting of one or more polypeptides substantially encoded (defined below) by all or part of the recognized antibody genes. The recognized immunoglobulin genes include, but are not limited to, the kappa, lambda, alpha, gamma (IgG1, IgG2, IgG3, and IgG4), delta, epsilon and mu constant region genes, as well as the myriad immunoglobulin variable region genes. Antibody herein is meant to include full-length antibodies and antibody fragments, and include antibodies that exist naturally in any organism or are engineered (e.g. are variants). By “antibody fragment” is meant any form of an antibody other than the full-length form. Antibody fragments herein include antibodies that are smaller components that exist within full-length antibodies, and antibodies that have been engineered. Antibody fragments include but are not limited to Fv, Fc, Fab, and (Fab′)₂, single chain Fv (scFv), diabodies, triabodies, tetrabodies, bifunctional hybrid antibodies, and the like (Maynard & Georgiou, 2000, Annu. Rev. Biomed. Eng. 2:339-76; Hudson, 1998, Curr. Opin. Biotechnol. 9:395-402). By “amino acid” and “amino acid identity” as used herein is meant one of the 20 naturally occurring or any non-natural analogues that may be present at a specific, defined position. By “computational screening method” herein is meant any method for designing one or more mutations in a protein, wherein said method utilizes a computer to evaluate the energies of the interactions of potential amino acid side chain substitutions with each other and/or with the rest of the protein. By “experimental library” herein is meant a list of one or more protein variants, existing either as a list of amino acid sequences or a list of the nucleotides sequences encoding them. Description of an experimental library may be defined, meaning that variant sequences are expressly described. Description of an experimental library may also be combinatorial, meaning that possible amino acid identities are indicated at variable positions, and the combination of all possibilities at all variable positions results in an expanded, explicitly defined library. By “Fc” herein is meant the polypeptides of an antibody that are comprised of immunoglobulin domains Cgamma2 and Cgamma3 (Cγ2 and Cγ3). Fc may also include any residues which exist in the N-terminal hinge between Cγ2 and Cgamma1 (Cγ1). These regions are shown in FIG. 1. Fc may refer to this region in isolation, or this region in the context of an antibody or antibody fragment. By “full-length antibody” herein is meant the structure that constitutes the natural biological form of an antibody. In most mammals, including humans, and mice, this form is a tetramer and consists of two identical pairs of two immunoglobulin chains, each pair having one light and one heavy chain, each light chain comprising immunoglobulin domains V_{L} and C_{L}, and each heavy chain comprising immunoglobulin domains V_{H}, Cγ1, Cγ2, and Cγ3. In each pair, the light and heavy chain variable regions (V_{L} and V_{H}) are together responsible for binding to an antigen, and the constant regions (C_{L}, Cγ1, Cγ2, and Cγ3, particularly Cγ2, and Cγ3) are responsible for antibody effector functions. In some mammals, for example in camels and llamas, full-length antibodies may consist of only two heavy chains, each heavy chain comprising immunoglobulin domains V_{H}, Cγ2, and Cγ3. By “immunoglobulin (Ig)” herein is meant a protein consisting of one or more polypeptides substantially encoded by immunoglobulin genes. Immunoglobulins include but are not limited to antibodies. Immunoglobulins may have a number of structural forms, including but not limited to full-length antibodies, antibody fragments, and individual immunoglobulin domains including but not limited to V_{H}, Cγ1, Cγ2, Cγ3, V_{L}, and C_{L}. By “immunoglobulin (Ig) domain” herein is meant a protein domain consisting of a polypeptide substantially encoded by an immunoglobulin gene. Ig domains include but are not limited to V_{H}, Cγ1, Cγ2, Cγ3, V_{L}, and C_{L} as is shown in FIG. 1. By “position” as used herein is meant a location in the sequence of a protein. Positions are typically, but not always, numbered sequentially. For example, position 297 is a position in the human antibody IgG1. By “residue” as used herein is meant a position in a protein and its associated amino acid identity. For example, Asparagine 297 (or Asn297 or N297) is a residue in the human antibody IgG1. By “variant protein sequence” as used herein is meant a protein sequence that has one or more residues that differ in amino acid identity from another similar protein sequence. Said similar protein sequence may be the natural wild type protein sequence, or another variant of the wild type sequence. In general, a starting sequence is referred to as a “parent” sequence, and again may either be a wild type or variant sequence. For example, preferred embodiments of the present invention may utilized humanized parent sequences upon which computational analyses are done. By “variable region” of an antibody herein is meant a polypeptide or polypeptides composed of the V_{H} immunoglobulin domain, the V_{L} immunoglobulin domains, or the V_{H} and V_{L} immunoglobulin domains as is shown in FIG. 1 (including variants). Variable region may refer to this or these polypeptides in isolation, as an Fv fragment, as an scFv fragment, as this region in the context of a larger antibody fragment, or as this region in the context of a full-length antibody.

The present invention may be applied to antibodies obtained from a wide range of sources. The antibody may be substantially encoded by an antibody gene or antibody genes from any organism, including but not limited to humans, mice, rats, rabbits, camels, llamas, dromedaries, monkeys, particularly mammals and particularly human and particularly mice and rats. In a preferred embodiment, the antibody is fully human, obtained for example using transgenic mice or other animals (Bruggemann & Taussig, 1997, Curr. Opin. Biotechnol. 8:455-458) or human antibody libraries coupled with selection methods (Griffiths & Duncan, 1998, Curr. Opin. Biotechnol. 9:102-108). The antibody does not necessarily need to be naturally occurring. For example the present invention could be used to optimize an engineered antibody, including but not limited to chimeric antibodies and humanized antibodies (Clark, 2000, Immunol. Today 21:397-402). In addition, the antibody being optimized may be an engineered variant of an antibody that is substantially encoded by one or more natural antibody genes. For example, in a one embodiment the antibody being optimized is an antibody that has been affinity matured.

In general, the computationally generated antibody genes of the present invention are designed to be substantially encoded by a naturally occurring antibody gene such as a humanized antibody gene. “Substantially encoded” can include a number of components, including host cell codon usage and complementarity to wild type genes. For example, in one embodiment, “substantially encoded” can be defined as the ability of the computationally generated gene being sufficiently complementary to the wild type gene (or its complement, depending on sense and antisense considerations) such that hybridization can occur. This complementarily need not, and is preferably not perfect; that is, due to the alteration of the variable residues, there are a number of substitutions (and sometimes insertions or deletions) between the two sequences that result in differences between the sequences. However, if the number of mutations is so great that no hybridization can occur under even the least stringent of hybridization conditions, the sequence is not a complementary sequence. Thus, by “substantially complementary” herein is meant that the sequences are sufficiently complementary to each other to hybridize under the selected reaction conditions. High stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10 C lower than the thermal melting point (Tm) for the specific sequence at a defined ionic strength pH. The Tm is the temperature (under defined ionic strength, pH and nucleic acid concentration) at which 50% of the probes complementary to the target hybridize to the target sequence at equilibrium (as the target sequences are present in excess, at Tm, 50% of the probes are occupied at equilibrium). Stringent conditions will be those in which the salt concentration is less than about 1.0 M sodium ion, typically about 0.01 to 1.0 M sodium ion concentration (or other salts) at pH 7.0 to 8.3 and the temperature is at least about 30 C for short probes (e.g. 10 to 50 nucleotides) and at least about 60 C for long probes (e.g. greater than 50 nucleotides). Stringent conditions may also be achieved with the addition of destabilizing agents such as formamide. In another embodiment, less stringent hybridization conditions are used; for example, moderate or low stringency conditions may be used, as are known in the art; see Maniatis and Ausubel, supra, and Tijssen, supra.

In another embodiment, “substantially encoded” means that at least a significant portion of the gene is identical to the parent gene such as a humanized or human antibody. In preferred embodiments, there are large areas of perfect complementarity punctuated by the variant positions which may be different. In preferred embodiments, at least 75% of the total gene is encoded by the parent gene, with at least 85%, 90%, 95% and 98% being preferred.

The present invention may be applied to a wide range of antibody structural forms. For example, the antibody may be a full-length antibody, an antibody fragment, an Fc region, a variable region, an individual immunoglobulin domain, or a structural motif, site, or loop of an antibody. The antibody may comprise more than one protein chain. That is, the antibody may be an oligomer, including a homo- or hetero-oligomer.

The present invention may be applied to a wide range of antibody products. In one embodiment the antibody product is a therapeutic, a diagnostic, or a research reagent. In a preferred embodiment the antibody product is a therapeutic antibody which may be used to treat disease, such diseases including, but not limited to cancer, autoimmune disease, cardiovascular disease, and the like. The antibody product may find use in a composition that is monoclonal or polyclonal, and that could be injected intravenously, subcutaneously, intramuscularly, and the like, as well as inhaled, applied topically, or via an oral dosage form, or otherwise administered. In an alternate embodiment, the antibody product is a library that could be screened experimentally, for example to generate antibodies against a target antigen using a selection method as described herein, or to affinity mature a particular antibody. This library may be a theoretical library, that is a list of nucleic acid or amino acid sequences, or may be a physical library of nucleic acids or proteins that encode the library sequences.

Computational Screening Methodology

A three-dimensional structure of an antibody is used as the starting point of the computational screening method of the present invention. The positions to be optimized are identified, which may be the entire antibody sequence or subset(s) thereof. Amino acids that will be considered at each position are selected. In a preferred embodiment, each considered amino acid may be represented by a discrete set of allowed conformations, called rotamers. Interaction energies are calculated between each considered amino acid and 1) each other considered amino acid, and 2) the rest of the protein, including the protein backbone and invariable residues. In a preferred embodiment, interaction energies are calculated between each considered amino acid side chain rotamer and 1) each other considered amino acid side chain rotamer and 2) the rest of the protein, including the protein backbone and invariable residues. One or more combinatorial search algorithms are then used to identify the lowest energy sequence and/or low energy sequences that will comprise an experimental library.

In a preferred embodiment, the computational screening method used to optimize antibodies is Protein Design Automation® (PDA™) technology, as is described in U.S. Pat. Nos. 6,188,965; 6,269,312; and 6,403,312; U.S. Ser. Nos. 09/782,004; 09/927,790; and 10/218,102; PCTs 98/07254; 01/40091; and 02/25588, all of which are expressly incorporated herein by reference. In another preferred embodiment, a Sequence Prediction Algorithm (SPA) is used to design proteins that are compatible with a known protein backbone structure as is described in Raha, et al., 2000, Protein Sci. 9:1106-1119, U.S. Ser. Nos. 09/877,695 and 10/071,859, all expressly incorporated herein by reference. In some embodiments, combinations of different computational screening methods are used, including combinations of PDA™ and SPA, as well as combinations of these computational techniques in combination with sequence and structural alignment. Similarly, these computational methods can be used simultaneously or sequentially, in any order. Furthermore, these computational methods can be used with experimental methods (shuffling, error-prone PCR, etc.) as outlined below. It is also important to note that reiterative cycles are included; thus for example, a first computational step may be done, followed by some experimental techniques, followed by additional computational techniques.

Computational screening, viewed broadly, has four steps: 1) selection and preparation of the antibody template or templates, 2) selection of variable positions and considered amino acids at those positions, and in a preferred embodiment selection of rotamers to model amino acids, 3) energy calculation, and 4) combinatorial optimization. As will be appreciated by those skilled in the art, energy calculation and combinatorial optimization are the computationally intensive aspects of computational screening, and together these two steps are referred to as design calculations.

Selection and Preparation of the Antibody Template

By “template antibody” herein is meant the structural coordinates of part or all of an antibody to be optimized. The template antibody is used as input in the computational screening calculations. A template protein may be part or all of any protein that has a known structure or for which a structure may be calculated, estimated, modeled or determined experimentally.

The template protein may be any antibody for which a three dimensional structure (that is, three dimensional coordinates for a set of the protein's atoms) is known or may be generated. The three dimensional structures of antibodies may be determined using methods including but not limited to X-ray crystallographic techniques, nuclear magnetic resonance (NMR) techniques, de novo modeling, and homology modeling. Antibody/antigen complexes may also be obtained using docking methods. Suitable antibody structures include, but are not limited to, all of those found in the Protein Data Base compiled and serviced by the Research Collaboratory for Structural Bioinformatics (RCSB, formerly the Brookhaven National Lab).

As will be appreciated by those skilled in the art, antibodies are a family of proteins that are closely related in sequence and structure. Consequently, homology models, which are generated using available sequence and structure information from other antibodies, are often of high quality. Thus, if optimization is desired for an antibody for which the structure has not been solved experimentally, a suitable structural model may be generated that may serve as the template for design calculations. Methods for generating homology models are known in the art. Methods for generating homology models of proteins are known in the art, and these methods find use in the present invention. See for example, Luo, et al. 2002, Protein Sci. 11:1218-1226, Lehmann & Wyss, 2001, Curr. Opin. Biotechnol. 12(4):371-5.; Lehmann et al., 2000, Biochim Biophys Acta. 1543(2):408-415; Rath & Davidson, 2000, Protein Sci., 9(12):2457-69; Lehmann et al., 2000, Protein Eng. 13(1):49-57; Desjarlais & Berg, 1993, Proc Natl Acad Sci USA. 90(6):2256-60; Desjarlais & Berg, 1992, Proteins. 12(2):101-4; Henikoff & Henikoff, 2000, Adv. Protein Chem. 54:73-97; Henikoff & Henikoff, 1994, J. Mol. Biol. 243(4):574-8; all herein expressly incorporated by reference. Methods for generating homology models of antibodies in particular are described in Morea et al., 2000, Methods 20:267-269, all herein expressly incorporated by reference.

As discussed above, the template may comprise any of a number of antibody structural forms. The template used in antibody design calculations may comprise an entire full-length antibody, a subset of an antibody such as a fragment, an individual immunoglobulin domain, or a structural motif, site, or loop of an antibody. The template antibody may comprise more than one protein chain, and may be the complex of an antibody bound to its antigen or to an antibody receptor. The template may additionally contain nonprotein components, including but not limited to small molecules, substrates, cofactors, metals, water molecules, prosthetic groups, polymers and carbohydrates. As will be appreciated by those in the art, the target antigen of an antibody may be a protein or a non-protein molecule. In a preferred embodiment, the structural template is a plurality or set of template proteins, for example or an ensemble of structures such as those obtained from NMR. Alternatively, the set of antibody templates is generated from a set of related proteins or structures, or artificially created ensembles.

The protein template may be modified or altered prior to design calculations. A variety of methods for template preparation are described in U.S. Pat. Nos. 6,188,965; 6,269,312; and 6,403,312; U.S. Ser. Nos. 09/782,004; 09/927,790; 09/877,695; 10/071,859 and 10/218,102; PCTs 98/07254; 01/40091; and 02/25588, all of which are herein expressly incorporated by reference. For example, in a preferred embodiment, explicit hydrogens may be added if not included within the structure. In a preferred embodiment, energy minimization of the structure is run to relax strain, including strain due to van der Waals clashes, unfavorable bond angles, and unfavorable bond lengths. Alternatively, the protein template is altered using other methods, such as manually, including directed or random perturbations. It is also possible to modify the protein template during later steps of a design calculation, including during the energy calculation and combinatorial optimization steps. In an alternate embodiment, the protein template is not modified before or during design calculations.

Selection of Variable Positions and Considered Amino Acids

Selection of Variable, Floated, and Fixed Positions

As is known in the art, it may be beneficial to reduce the complexity of a calculation by allowing mutation only at certain variable positions. By “variable position” herein is meant a position at which the amino acid identity is allowed to be altered in a design calculation. In a preferred embodiment the amino acid identity to which a position may be mutated is the full set or a subset of the 20 naturally occurring amino acids. Alternatively, variable positions may be allowed to mutate to a set of non-naturally occurring amino acids or synthetic analogs. One or more residues may be variable positions in design calculations.

Residues that are chosen as variable positions may be those that contribute to or are hypothesized to contribute to the antibody property to be optimized. For the present invention, these properties include stability, solubility, and affinity for antigen. Residues at variable positions may contribute favorably or unfavorably to a specific antibody property. For example, a residue at the antibody/antigen interface may be involved in mediating binding with antigen, and thus this position may be varied in design calculations aimed at improving affinity with antigen. Alternatively, as another example, a residue which has an exposed hydrophobic side chain may be responsible for causing unfavorable aggregation, and thus this position may be varied in design calculations aimed a improving solubility.

Thus in one embodiment, variable positions may be those positions that are directly involved in interactions that are determinants of an antibody property. For example, the antigen binding site of an antibody may be defined to include all residues that contact antigen. By “contact” herein is meant some chemical interaction between at least one atom of an antibody residue with at least one atom of the bound antigen, with chemical interaction including, but not limited to van der Waals interactions, hydrogen bond interactions, electrostatic interactions, and hydrophobic interactions. In an alternative embodiment, variable positions may include those positions that are indirectly involved in an antibody property, i.e. such positions may be proximal to residues that contribute to an antibody property. For example, the antigen binding site of an antibody may be defined to include all residues within a certain distance, for example 4-10 Å, of the residues that are in van der Waals contact with antigen. Thus variable positions in this case may be chosen not only as residues that directly contact antigen, but also those that contact residues that contact antigen and thus influence antigen binding indirectly. The specific positions chosen are dependent on the design strategy being employed.

In a preferred embodiment, some of the residue positions that are not variable are floated. By “floated position” herein is meant a position at which the amino acid conformation but not the amino acid identity is allowed to vary in a protein design calculation. In one embodiment the floated position may have the wild type amino acid identity. For example, floated positions may be wild type positions that are within a small distance of, for example, 5 Å, of a variable position residue. In an alternate embodiment, a floated position may have a non-wild type amino acid identity. Such an embodiment may find use in the present invention, for example, when the goal is to evaluate the energetic or structural outcome of a specific mutation.

Residue positions that are not variable or floated are fixed. By “fixed position” herein is meant a position at which the amino acid identity and the conformation are held constant in a protein design calculation. Residues, which may be fixed, may include residues that are not involved or not thought to be involved in the property to be optimized. In this case there is nothing to be gained by varying these positions. Residues that may be fixed may also include but are not limited to residues that are important for maintaining proper folding, structure, stability, solubility, and biological function. For example, residues that interact with protein receptors or residues that are glycosylation sites may be fixed in design calculations to ensure that receptor binding and proper glycosylation respectively are not perturbed. Likewise, if stability is being optimized, it may be beneficial to fix residues that directly or indirectly interact with antigen so that antigen binding is not perturbed. Fixed positions may also include structurally important residues such as cysteines participating in disulfide bridges, residues critical for backbone conformation such as proline or glycine, critical hydrogen bonding residues, and residues that form favorable packing interactions.

Selection of Amino Acids to be Considered at Each Position

The next step in the computational screening method of the present invention is to select a set of possible amino acid identities that will be considered at each particular variable position. This set of possible amino acids is herein referred to as “considered amino acids” at a variable position. In one embodiment, all 20 amino acids (or their analogues or synthetic amino acids) are considered at a given variable position. Alternatively, a subset of amino acids, or even only one amino acid is considered at a given variable position. As will be appreciated by those skilled in the art, there is a computational benefit to considering only certain amino acid identities at variable positions, as it decreases the combinatorial complexity of the search. Furthermore, considering only certain amino acids at variable positions may be used to tune calculations toward specific design strategies. For example, for solubility optimization, it may be beneficial to allow only polar amino acids to be considered at surface exposed variable positions. In a preferred embodiment for solubility, at least one antibody sequence possesses an increase in polar character. Alternatively preferred, is selecting at least one nonpolar amino acid and substituting said nonpolar amino acid with a polar amino acid.

A wide variety of methods may be used, alone or in combination, to select which amino acids will be considered at each position, including but not limited to those discussed below.

For example, as is known in the art, the set of amino acids allowed at variable positions may be chosen based on the degree of exposure to solvent. Hydrophobic or nonpolar amino acids typically reside in the interior or core of a protein, which are inaccessible or nearly inaccessible to solvent. Thus at variable core positions it may be beneficial to consider only or mostly nonpolar amino acids such as alanine, valine, isoleucine, leucine, phenylalanine, tyrosine, tryptophan, and methionine. Hydrophilic or polar amino acids typically reside on the exterior or surface of proteins, which have a significant degree of solvent accessibility. Thus at variable surface positions it may be beneficial to consider only or mostly polar amino acids such as alanine, serine, threonine, aspartic acid, asparagine, glutamine, glutamic acid, arginine, lysine and histidine. Some positions are partly exposed and partly buried, and are not clearly protein core or surface positions, in a sense serving as boundary residues between core and surface residues. Thus at such variable boundary positions it may be beneficial to consider both nonpolar and polar amino acids such as alanine, serine, threonine, aspartic acid, asparagine, glutamine, glutamic acid, arginine, lysine histidine, valine, isoleucine, leucine, phenylalanine, tyrosine, tryptophan, and methionine.

Determination of the degree of solvent exposure at variable positions may be by subjective evaluation or visual inspection of the antibody template by one skilled in the art of protein structural biology, or by the use of a variety of algorithms that are known in the art. Selection of amino acid types to be considered at variable positions may be aided or determined wholly by computational methods, such as calculation of solvent accessible surface area, or using algorithms which assess the orientation of the Calpha-Cbeta vectors relative to a solvent accessible surface, as outlined in U.S. Pat. Nos. 6,188,965; 6,269,312; and 6,403,312; U.S. Ser. Nos. 09/782,004; 09/927,790; and 10/218,102; PCTs 98/07254; 01/40091; and 02/25588, and expressly herein incorporated by reference. In an embodiment, each variable position may be classified explicitly as a core, surface, or boundary position.

In an alternate embodiment, selection of the set of amino acids allowed at variable positions may be hypothesis-driven. Hypotheses for which amino acid types should be considered at variable positions may be derived by a subjective evaluation or visual inspection of the antibody template by one skilled in the art of protein structural biology. For example, if it is suspected that a hydrogen bonding interaction may be favorable at a variable position, polar residues that have the capacity to form hydrogen bonds may be considered even if the position is in the core. Likewise, if it is suspected that a hydrophobic packing interaction may be favorable at a variable position, nonpolar residues that have the capacity to form favorable packing interactions may be considered even if the position is on the surface. Other examples of hypothesis-driven approaches may involve issues of backbone flexibility or protein fold. As is known in the art, certain residues, for example proline, glycine, and cysteine, play important roles in protein structure and stability. Glycine enables greater backbone flexibility than all other amino acids, proline constrains the backbone more than all other amino acids, and cysteines may form disulfide bonds. It may therefore be beneficial to include one or more of these amino acid types to achieve a desired goal. Alternatively, it may be beneficial to exclude one or more of these amino acid types from the list of considered amino acids.

In an alternate embodiment, subsets of amino acids may be chosen to maximize coverage. In this case, additional amino acids with properties similar to that in the antibody template may be considered at variable positions. For example, if the residue at a variable position in the antibody template is a large hydrophobic residue, the user may choose to include additional large hydrophobic amino acids at that position. Alternatively, subsets of amino acids may be chosen to maximize diversity. In this case, amino acids with properties dissimilar to those in the antibody template may be considered at variable positions. For example, if the residue at a variable position in the antibody template is a large hydrophobic residue, the user may choose to include only one large hydrophobic amino acid in combination with other amino acids that are small, polar, etc.

Selection of Rotamers to Model Amino Acids

As is known in the art, some computational screening methods require only the identity of considered amino acids to be determined during design calculations. That is, no information is required concerning the conformations or possible conformations of the amino acid side chains. As is also known in the art, and in a preferred embodiment, a set of discrete side chain conformations, called rotamers, can be considered for each amino acid. Thus, a set of rotamers will be considered at each variable and floated position. Rotamers may be obtained from published rotamer libraries (see for example, Lovel et al., 2000, Proteins: Structure Function and Genetics 40:389-408; Dunbrack & Cohen, 1997, Protein Science 6:1661-1681; DeMaeyer et al., 1997, Folding and Design 2:53-66; Tuffery et al., 1991, J. Biomol. Struct. Dyn. 8:1267-1289, Ponder & Richards, 1987, J. Mol. Biol. 193:775-791). As is known in the art, rotamer libraries may be backbone-independent or backbone-dependent. Rotamers may also be obtained from molecular mechanics or ab initio calculations, and using other methods. In a preferred embodiment, a flexible rotamer model is used (see Mendes et. al., 1999, Proteins: Structure, Function, and Genetics 37:530-543). Similarly, artificially generated rotamers may be used, or augment the set chosen for each amino acid and/or variable position. In a preferred embodiment, at least one conformation that is not low in energy is included in the list of rotamers. In an alternatively preferred embodiment, the rotamer of the variable position residue in the antibody template is included in the list of rotamers allowed for that variable position in the design calculation. In an alternative embodiment, only the identity of each amino acid considered at variable positions is provided, and no specific conformational states of each amino acid are used during design calculations. That is, use of rotamers is not essential for computational screening.

Use of Experimental Information

In one embodiment of the present invention, experimental information may be used to guide the choice of variable positions, and/or the choice of considered amino acids at variable positions. As is known in the art, mutagenesis experiments are often carried out to determine the role of certain residues in protein structure and function, for example, which protein residues play a role in determining stability, or which residues make up the antigen binding site of an antibody. Data obtained from such experiments are useful in the present invention.

For example, variable positions for affinity maturation calculation could involve varying all positions at which mutation has been shown to affect binding. Similarly, the results from such an experiment may be used to guide the choice of allowed amino acid types at variable positions. For example, if certain types of amino acid substitutions are found to be favorable, sets, subsets, and/or similar types of those amino acids may be chosen to maximize coverage. In one embodiment, additional amino acids with properties similar to that or those that were found to be favorable experimentally may be considered at variable positions. For example, if experimental mutation of a variable position residue at the antigen interface to a large hydrophobic residue was found to be favorable, the user may choose to include additional large hydrophobic amino acids at that position in the computational screen.

As is known in the art, display and other selection technologies may be coupled with random mutagenesis to generate a list or lists of amino acid substitutions that are favorable for the selected property. Such a list or lists obtained from such experimental work find use in the present invention. For example, positions that are found to be invariable in such an experiment may be excluded as variable positions in computational screening calculations, whereas positions that are found to be more acceptable to mutation or respond favorably to mutation may be chosen as variable positions. Similarly, the results from such experiments may be used to guide the choice of allowed amino acid types at variable positions. For example, if certain types of amino acids arise more frequently in an experimental selection, subsets or similar types of those amino acids may be chosen to maximize coverage. In one embodiment, additional amino acids with properties similar to those that were found to be favorable experimentally may be considered at variable positions. For example, if selected mutations at a variable position that resides at the antigen interface are found to be uncharged polar amino acids, the user may choose to include additional uncharged polar amino acids, or perhaps charged polar amino acids, at that position.

Use of Sequence Information

In one embodiment of the present invention, sequence information may be used to guide choice of variable positions, and/or the choice of amino acids considered at variable positions. As is known in the art, all antibodies share a common structural scaffold and are homologous in sequence. Furthermore, there is a large amount of sequence and structural information available for the antibody family of proteins. These favorable aspects of antibodies may be used to gain insight into particular positions in the antibody family. As is known in the art, sequence alignments are often carried out to determine which antibody residues are conserved and which are not conserved. That is to say, by comparing and contrasting alignments of antibody sequences, the degree of variability at a position may be observed, and the types of amino acids that occur naturally at positions may be observed. Data obtained from such analyses are useful in the present invention.

The benefit of using sequence information to choose variable positions and considered amino acids at variable positions are several fold. For choice of variable positions, the primary advantage of using sequence information is that insight may be gained into which positions are more tolerant and which are less tolerant to mutation. Thus sequence information may aid in ensuring that quality diversity, i.e. mutations that are not deleterious to protein structure, stability, etc., is sampled computationally. The same advantage applies to use of sequence information to select amino acid types considered at variable positions. That is, the set of amino acids which occur in an antibody sequence alignment may be thought of as being pre-screened by evolution to have a higher chance than random for being compatible with an antibody's structure, stability, solubility, function, etc. Thus higher quality diversity is sampled computationally. A second benefit of using sequence information to select amino acid types considered at variable positions is that certain alignments may represent sequences that may be less immunogenic than random sequences. For example, if the amino acids considered at a given variable position are the set of amino acids which occur at that position in an alignment of human germ line antibody sequences, those amino acids may be thought of as being pre-screened by nature for generating no or low immune response if the optimized antibody is used as a human therapeutic.

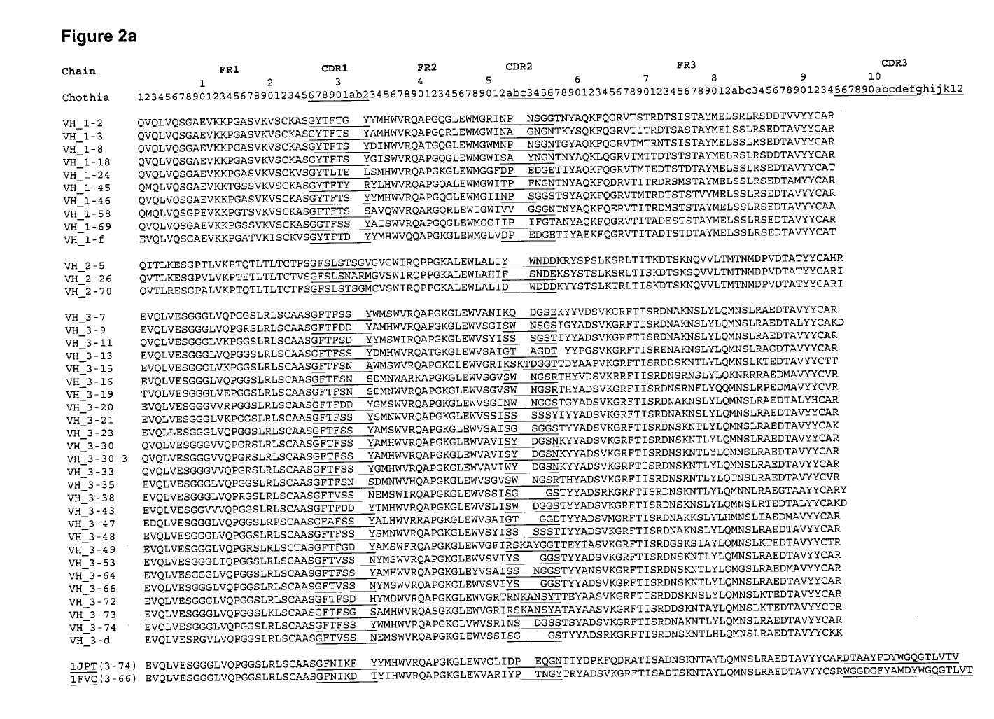

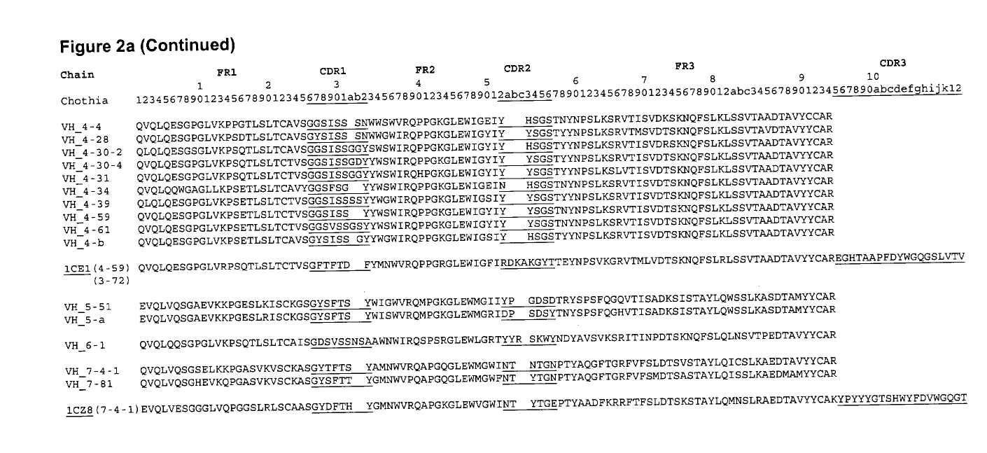

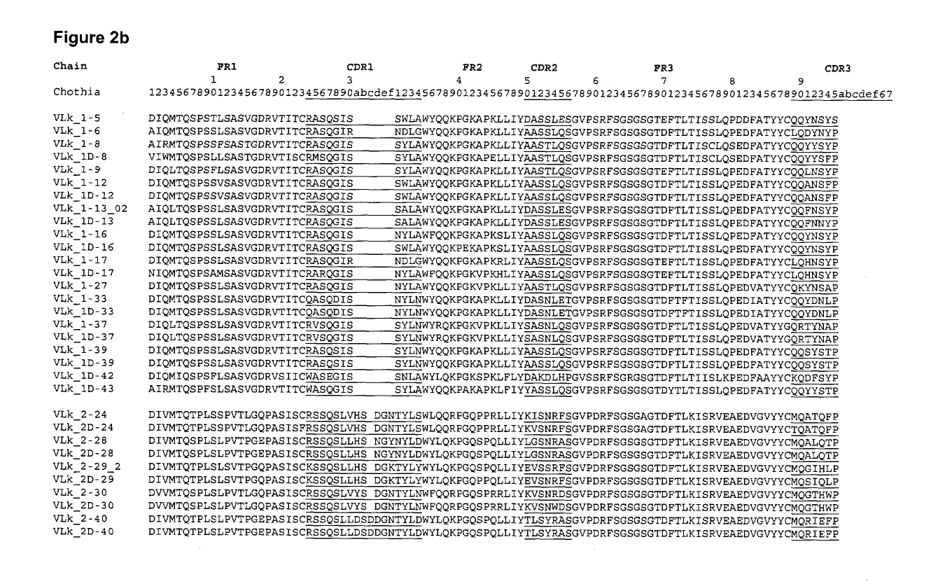

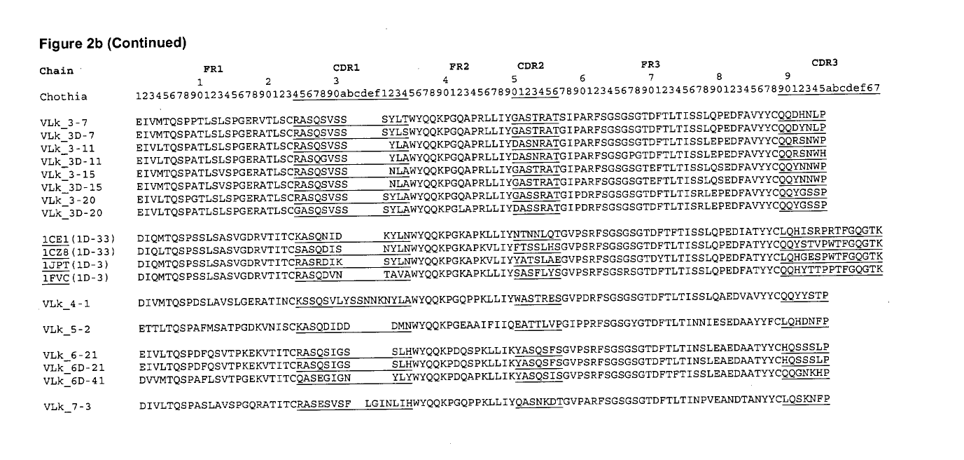

The source of the sequences may vary widely, and include one or more of the known databases, including but not limited to the Kabat database (.immuno.bme.nwu.edu; Johnson & Wu, 2001, Nucleic Acids Res. 29:205-206; Johnson & Wu, 2000, Nucleic Acids Res. 28:214-218), the IMGT database (IMGT, the international ImMunoGeneTics information system®; imgt.cines.fr; Lefranc et al., 1999, Nucleic Acids Res. 27:209-212; Ruiz et al., 2000 Nucleic Acids Res. 28:219-221; Lefranc et al., 2001, Nucleic Acids Res. 29:207-209; Lefranc et al., 2003, Nucleic Acids Res. 31:307-310), and VBASE (.mrc-cpe.cam.ac.uk/vbase-ok.php?menu=901). Antibody sequence information can be obtained, compiled, and/or generated from sequence alignments of germ line sequences or sequences of naturally occurring antibodies from any organism, including but not limited to mammals. For example, FIGS. 2a and 2b list the aligned human V_{H} and V_{L} kappa germ line sequences, along with several antibody variable region sequences relevant to the examples of the present invention. Alternatively, antibody sequence information can be obtained from a database that is compiled privately. Other databases which are more general nucleic acid or protein databases, i.e. not particular to antibodies, for example including but are not limited to SwissProt (expasy.ch/sprot/), GenBank (ncbi.nlm.nih.gov/Genbank) and Entrez (ncbi.nlm.nih.gov/Entrez/), and EMBL Nucleotide Sequence Database (ebi.ac.uk/embl/), may find use in the present invention. There are numerous sequence-based alignment programs and methods known in the art, and all of these find use in the present invention for generation of antibody sequence alignments.

Once alignments are made, sequence information can be used to guide choice of variable positions. Such sequence information can relate the variability, natural or otherwise, of a given position. Variability herein should be distinguished from variable position. By “variability” herein is meant the degree to which a given position in a sequence alignment shows variation in the types of amino acids that occur there. Variable position, to reiterate, is a position chosen by the user to vary in amino acid identity during a computational screening calculation. Variability may be determined qualitatively by one skilled in the art of bioinformatics. There are also methods known in the art to quantitatively determine variability that may find use in the present invention. The most preferred embodiment measures Information Entropy or Shannon Entropy. Variable positions can be chosen based on sequence information obtained from closely related antibody sequences, or antibody sequences that are less closely related.

The use of sequence information to choose variable positions finds broad use in the present invention. For example, to optimize antibody solubility by replacing exposed nonpolar surface residues, variable positions may be chosen as only that set of surface exposed positions that show a certain level of variability. As another example, to optimize antibody stability by mutating interdomain interface residues, variable positions may be chosen as only that set of interface positions that shown a certain level of variability. For example, if an interface position in the antibody template is tryptophan, and tryptophan is observed at that position in greater than 90% of the sequences in an alignment, it may be beneficial to leave that position fixed. In contrast, if another interface position is found to have a greater level of variability, for example if five different amino acids are observed at that position with frequencies of approximately 20% each, that position may be chosen as a variable position. In another embodiment, variable positions for affinity maturation calculations could be chosen to be all positions or a subset of positions which are determined by sequence alignment to make up a complementarity determining region (CDR) loop. Alternatively, variable positions could be chosen to be those residues that are determined by sequence alignment to contact a CDR loop. Thus, visual inspection of an aligned antibody sequence may substitute for visual inspection of an antibody structure. This is due to the high level of both sequence and structural similarity in the antibody family. The rationale here is that those positions which typically contact a CDR in most antibody structures, for example, are hypothesized to be positions which contact a CDR in the antibody template being optimized in the calculation.

Sequence information can also be used to guide the choice of amino acids considered at variable positions. Such sequence information can relate to how frequently an amino acid, amino acids, or amino acid types (for example polar or nonpolar, charged or uncharged) occur, naturally or otherwise, at a given position. In one embodiment, the set of amino acids considered at a variable position in design calculations may comprise the set of amino acids that is observed at that position in the alignment. Thus, the position-specific alignment information is used directly to generate the list of considered amino acids at a variable position in a computational screening calculation. Such a strategy is well known in the art. See for example Lehmann & Wyss, 2001, Curr. Opin. Biotechnol. 12(4):371-5.; Lehmann et al., 2000, Biochim Biophys Acta. 1543(2):408-415; Rath & Davidson, 2000, Protein Sci., 9(12):2457-69; Lehmann et al., 2000, Protein Eng. 13(1):49-57; Desjarlais & Berg, 1993, Proc Natl Acad Sci USA. 90(6):2256-60; Desjarlais & Berg, 1992, Proteins. 12(2):101-4; Henikoff & Henikoff, 2000, Adv. Protein Chem. 54:73-97; Henikoff & Henikoff, 1994, J. Mol. Biol. 243(4):574-8; all herein expressly incorporated by reference.

In an alternate embodiment, the set of amino acids considered at a variable position or positions may comprise a set of amino acids that is observed most frequently in the alignment. Thus, a certain criteria is applied to determine whether the frequency of an amino acid or amino acid type will be included in the set of amino acids that are considered at a variable position in a design calculation. As is known in the art, sequence alignments may be analyzed using statistical methods to calculate the sequence diversity at any position in the alignment and the occurrence frequency or probability of each amino acid at a position. Such data may then be used to determine which amino acids types to consider. In the simplest embodiment, these occurrence frequencies are calculated by counting the number of times an amino acid is observed at an alignment position, then dividing by the total number of sequences in the alignment. In other embodiments, the contribution of each sequence, position or amino acid to the counting procedure is weighted by a variety of possible mechanisms. In a preferred embodiment, the contribution of each aligned sequence to the frequency statistics is weighted according to its diversity weighting relative to other sequences in the alignment. A common strategy for accomplishing this is the sequence weighting system recommended by Henikoff and Henikoff (see Henikoff & Henikoff, 2000, Adv. Protein Chem. 54:73-97; Henikoff & Henikoff, 1994, J. Mol. Biol. 243:574-8; both herein expressly incorporated by reference. In a preferred embodiment, the contribution of each sequence to the statistics is dependent on its extent of similarity to the target sequence, i.e. the antibody template used in the design calculations, such that sequences with higher similarity to the target sequence are weighted more highly. Examples of similarity measures include, but are not limited to, sequence identity, BLOSUM similarity score, PAM matrix similarity score, and Blast score. In an alternate embodiment, the contribution of each sequence to the statistics is dependent on its known physical or functional properties. These properties include, but are not limited to, thermal and chemical stability, contribution to activity, solubility, etc. For example, when optimizing an antibody for solubility, those sequences in an alignment that are known to be most soluble (for example see Ewert et a., 2003, J. Mol.Biol. 325:531-553), will contribute more heavily to the calculated frequencies.

Regardless of what criteria are applied for choosing the set of amino acids in a sequence alignment to be considered at variable positions, using sequence information to choose the set of amino acids considered at variable positions finds broad use in the present invention. For example, to optimize antibody solubility by replacing exposed nonpolar surface residues, considered amino acids may be chosen as the set of amino acids, or a subset of those amino acids which meet some criteria, that are observed at that position in an alignment of antibody sequences. As another example, to optimize antibody stability by mutating domain interface residues, considered amino acids may be chosen as the set of amino acids, or a subset of those amino acids that meet some criteria, that are observed at that position in an alignment of antibody sequences. In an alternate embodiment, one or more amino acids may be added or subtracted subjectively from a list of amino acids derived from a sequence alignment in order to maximize coverage. For example, additional amino acids with properties similar to those that are found in a sequence alignment may be considered at variable positions. For example, if an antigen binding position is observed to have uncharged polar amino acids in an antibody sequence alignment, the user may choose to include additional uncharged polar amino acids in an affinity maturation calculation, or perhaps charged polar amino acids, at that position.

In a preferred embodiment, sequence alignment is not used alone in the analysis step of the present invention; that is, sequence information is combined with energy calculation, as discussed below. For example, pseudo energies can be derived from sequence information to generate a scoring function. The use of a sequence-based scoring function may assist in significantly reducing the complexity of a calculation. However, as is appreciated by those skilled in the art, the use of a sequence-based scoring function alone may be inadequate because sequence information can often indicate misleading correlations between mutations that may in reality be structurally conflicting. Thus, in a preferred embodiment, a structure-based method of energy calculation is used, either alone or in combination with a sequence-based scoring function. That is, preferred embodiments do not rely on sequence alignment information alone as the analysis step.

Energy Calculation

Some method of scoring each amino acid substitution, herein referred to as energy calculation, is required for computational screening. As previously discussed, there are a variety of ways to represent amino acids in order to enable efficient energy calculation.

In a preferred embodiment, considered amino acids are represented as rotamers, as described previously, and the energy (or score) of interaction of each possible rotamer at each variable position, or at each variable and floated position, with the template and/or other rotamers, is calculated. It should be understood that the template in this case includes both the atoms of the protein structure backbone, as well as the atoms of any fixed residues, as well as non-protein atoms. In a preferred embodiment, two sets of interaction energies are calculated for each side chain rotamer at every position: the interaction energy between the rotamer and the template (the “singles” energy), and the interaction energy between the rotamer and all other possible rotamers at every other variable and floated position (the “doubles” energy). In an alternate embodiment, singles and doubles energies are calculated for fixed positions as well as for variable and floated positions.

In an alternate embodiment, considered amino acids are not represented as rotamers.

In one embodiment, molecular dynamics calculations may be used to computationally screen sequences by individually calculating mutant sequence scores.

Regardless of how amino acids are represented, the energies of interaction are measured by one or more scoring functions. A variety of scoring functions find use in the present invention for calculating energies. As will be appreciated by those skilled in the art, certain scoring functions are more compatible with certain types of methods for representing amino acids. For example, force fields are particularly well suited to score amino acid substitutions that are represented as rotamers. However, in order to not constrain the present invention to any particular application or theory of operation, a variety of scoring functions are presented that may find use in the present invention regardless of how amino acids are represented.

Scoring functions may include a number of potentials, herein referred to as the energy terms of a scoring function, including but are not limited to, a van der Waals potential scoring function, a hydrogen bond potential scoring function, an atomic solvation potential scoring function, a secondary structure propensity potential scoring function and an electrostatic potential scoring function. At least one energy term is used to score each variable or floated position, although the energy terms may differ depending on the position classification or other considerations.

A variety of scoring functions are described in U.S. Pat. Nos. 6,188,965; 6,269,312; and 6,403,312; U.S. Ser. Nos. 09/782,004; 09/927,790; 09/877,695; 10/071,859 and 10/218,102; PCTs 98/07254; 01/40091; and 02/25588, all of which are herein expressly incorporated by reference. As will be appreciated by those skilled in the art, a number of force fields, which are comprised of one or more energy terms, may serve as scoring functions. Force fields include, but are not limited to, ab initio or quantum mechanical force fields, semi-empirical force fields, and molecular mechanics force fields. In an alternate embodiment, scoring functions that are knowledge-based may be used. In an alternate embodiment, scoring functions that use statistical methods may find use in the present invention. These methods may be used to assess the match between a sequence and a three-dimensional protein structure, and hence may be used to score amino acid substitutions for fidelity to the protein structure.

In a preferred embodiment, additional energy terms may be included in the scoring function. For example, the above mentioned scoring functions may be modified to include terms including but not limited to torsional potentials, entropy potentials, additional solvation models including contact models, solvent exclusion models, and knowledge-based energies derived from protein sequence and/or structure statistics including but not limited to threading potentials, reference energies, pseudo energies, and sequence biases derived from sequence alignments (as discussed in the previous section). In a preferred embodiment, a scoring function is modified to include models for immunogenicity, such as functions derived from data on binding of peptides to MHC (Major Histocompatability Complex), that may be used to identify potentially immunogenic sequences (see U.S. Ser. Nos. 09/903,378; 10/039,170; 60/222,697 and U.S. Ser. No. to be determined, filed Jan. 8, 2003 and entitled “NOVEL PROTEIN WITH ALTERED IMMUNOGENICITY”; and PCT 01/21823; and 02/00165, all herein expressly incorporated by reference).

In one embodiment, as is known in the art, one or more scoring functions may be optimized or “trained” during the computational analysis, and then the analysis re-run using the optimized system. Such altered scoring functions may be obtained for example, by training a scoring function using experimental data.

In a preferred embodiment, the scoring functions used are one or more of the scoring functions which are described in U.S. Pat. Nos. 6,188,965; 6,269,312; and 6,403,312; U.S. Ser. Nos. 09/782,004; 09/927,790; 09/877,695; 10/071,859 and 10/218,102; PCTs 98/07254; 01/40091; and 02/25588, all herein expressly incorporated by reference. In an alternate embodiment, energy calculation is carried out using one or more of the methods described above in combination.

In the most preferred embodiment, a scoring function using more than one energy term is used. As will be appreciated by those skilled in the art, Ig domain stabilization using only a van der Waals potential (Looger & Hellinga, 2001, J. Mol. Biol. 307:429-445) or affinity maturation using only an electrostatic potential may be inadequate for accurately evaluating the complex interactions in an antibody and between an antibody and its antigen. In the most preferred embodiment, energies may be calculated using a force field containing energy terms describing van der Waals, salvation, electrostatic, hydrogen bond interactions and combinations thereof. In additional embodiments, additional energy terms include but are not limited to entropic terms, torsional energies, and knowledge-based energies.

Combinatorial Optimization

An important component of computational screening is the identification of one or more sequences that have a favorable score or are low in energy. In a preferred embodiment, all possible interaction energies are calculated prior to optimization. In an alternatively preferred embodiment, energies may be calculated as needed during optimization.

The need for a combinatorial optimization algorithm is illustrated by examining the number of possibilities that are considered in a typical design calculation. The discrete nature of rotamer sets allows a simple calculation of the number of possible rotameric sequences for a given design problem. A backbone of length n with m possible rotamers per position will have mⁿ possible rotamer sequences, a number which grows exponentially with sequence length. For very simple design calculations, it is possible to examine each possible sequence in order to identify the optimal sequence and/or one or more favorable sequences. However, for a typical design problem, the number of possible sequences (up to 10⁸⁰ or more) is sufficiently large that examination of each possible sequence is intractable. A variety of combinatorial optimization algorithms may then be used to identify the optimum sequence and/or one or more favorable sequences.

Combinatorial optimization algorithms may be divided into two classes: (1) those that are guaranteed to return the global minimum energy configuration if they converge, and (2) those that are not guaranteed to return the global minimum energy configuration, but which will always return a solution. Examples of the first class of algorithms include, but are not limited to, Dead-End Elimination (DEE) and Branch & Bound (B&B) (including Branch and Terminate) (Gordon & Mayo, 1999, Structure Fold. Des. 7:1089-98). Examples of the second class of algorithms include, but are not limited to, Monte Carlo (MC), self-consistent mean field (SCMF), Boltzmann sampling (Metropolis et al., 1953, J. Chem. Phys. 21:1087), simulated annealing (Kirkpatrick et al., 1983, Science, 220:671-680), genetic algorithm (GA) and Fast and Accurate Side-Chain Topology and Energy Refinement (FASTER (Desmet, et al., 2002, Proteins, 48:31-43). A combinatorial optimization algorithm may be used alone or in conjunction with another combinatorial optimization algorithm.

In one embodiment of the present invention, the strategy for applying a combinatorial optimization algorithm is to find the global minimum energy configuration. In an alternate embodiment, the strategy is to find one or more low energy or favorable sequences. In an alternate embodiment, the strategy is to find the global minimum energy configuration and then find one or more low energy or favorable sequences. For example, as outlined in U.S. Pat. No. 6,269,312 and PCT US98/07254, preferred embodiments utilize a Dead End Elimination (DEE) step, and preferably a Monte Carlo step. In other embodiments tabu search algorithms are used or combined with DEE and/or Monte Carlo, among other search methods (see Modern Heuristic Search Methods, edited by V. J. Rayward-Smith, et al., 1996, John Wiley & Sons Ltd., hereby expressly incorporated by reference in its entirety and also U.S. Ser. No. 10/218,102 and PCT 02/25588). In another preferred embodiment, a genetic algorithm may be used. See, U.S. Ser. Nos. 09/877,695 and 10/071,859, both herein expressly incorporated by reference. As another example, as is more fully described in U.S. Pat. Nos. 6,188,965; 6,269,312; and 6,403,312; U.S. Ser. Nos. 09/782,004; 09/927,790; and 10/218,102; PCTs 98/07254; 01/40091; and 02/25588, which are herein expressly incorporated by reference, the global optimum may be reached, and then further computational processing may occur, which generates additional optimized sequences.

In the simplest embodiment, design calculations are not combinatorial. That is, energy calculations are used to evaluate the amino acid substitutions individually at single variable positions. However, it is a more preferred embodiment in certain situations to combine design calculations and also to evaluate amino acid substitutions at more than one variable positions.

Library Generation

The output sequence or sequences from computational screening may be used to generate an experimental library. By “experimental library” herein is meant a list of one or more protein variants, existing either as a list of amino acid sequences or a list of the nucleotides sequences encoding them. Such a library may then be screened experimentally to single out superior members of antibody variants that are optimized for the desired property. As discussed above, computationally screened libraries have a number of benefits. Computationally generated libraries are significantly enriched in stable, properly folded, and functional sequences relative to randomly generated libraries. Because of the overlapping sequence constraints on antibody structure, stability, solubility, function, etc., a large number of the candidates in an experimental library occupy “wasted” sequence space. For example, a large fraction of sequence space encodes unfolded, misfolded, incompletely folded, partially folded, or aggregated proteins. In contrast, experimental libraries that are screened computationally are composed primarily of productive sequence space. As a result, computational screening increases the chances of identifying antibodies that are broadly optimized for stability, solubility, and affinity for antigen. In effect, computational screening yields an increased hit-rate, thereby decreasing the number of variants that must be screened experimentally. The term “experimental library” may refer to the set of optimized antibodies in any form. In one embodiment, the library is a list of nucleic acid or amino acid sequences, or a list of nucleic acid or amino acid substitutions at variable positions. For example, the examples used to illustrate the present invention below provide experimental libraries as amino acid substitutions at variable positions. In an alternate embodiment, the library is a physical library composed of nucleic acids that encode the optimized library sequences. Said nucleic acids may be the genes encoding the optimized antibodies, the genes encoding the optimized antibodies with any operably linked nucleic acids, or expression vectors encoding the library members together with any other operably linked regulatory sequences, selectable markers, fusion constructs, and/or other elements. For example, the experimental library may be a set of mammalian expression vectors that encode library members, the protein products of which may be subsequently expressed, purified, and screened experimentally. As another example, the experimental library may be a display library. Such a library could, for example, be composed of a set of expression vectors which encode library members operably linked to some fusion partner that enables phage display, ribosome display, yeast display, bacterial surface display, and the like. Such a library could be used, for example, to screen for antibodies against a target antigen, or to affinity mature a particular antibody. In an alternate embodiment, the library is a physical library that is comprised of the optimized antibody proteins, either in purified or unpurified form.

In one embodiment, an experimental library is a list of at least one sequence that are variant antibodies optimized for a desired property. For example see, Filikov et a., 2002, Protein Sci. 11:1452-1461 and Luo et al., 2002, Protein Sci 11:1218-1226. In an alternate embodiment, an experimental library may be defined as a combinatorial list, meaning that each a list of amino acid substitutions is designed for each variable position, with the implication that each substitution is to be combined with all other designed substitutions at all other variable positions. In this case, expansion of the combination of all possibilities at all variable positions results in a large explicitly defined library.

Selecting Sequences for the Experimental Library

As is known in the art, there are a variety of ways that an experimental library may be derived from the output of computational screening calculations. For example, methods of library generation described in U.S. Pat. No. 6,403,312; U.S. Ser. Nos. 09/782,004; 09/927,790; and 10/218,102; PCTs 01/40091; and 02/25588, herein expressly incorporated by reference, find use in the present invention.

In one embodiment, sequences scoring within a certain range of the global optimum sequence may be included in the library. For example, all sequences within 10 kcal/mol of the lowest energy sequence could be used as the experimental library. In an alternate embodiment, sequences scoring within a certain range of one or more local minima sequences may be used. In a preferred embodiment, the library sequences are obtained from a filtered set. Such a list or set may be generated by a variety of methods, as is known in the art, for example using an algorithm such as Monte Carlo, B&B, or SCMF. For example, the top 10³ or the top 10⁵ sequences in the filtered set may comprise the experimental library. Alternatively, the total number of sequences defined by the combination of all mutations may be used as a cutoff criterion for the experimental library. Preferred values for the total number of recombined sequences range from 10 to 10²⁰, particularly preferred values range from 100 to 10⁹. Alternatively, a cutoff may be enforced when a predetermined number of mutations per position is reached.

Clustering algorithms may be useful for classifying sequences derived by computational screening methods into representative groups. For example, methods of clustering and their application described in U.S. Ser. No. 10/218,102 and PCT 02/25588, herein expressly incorporated by reference, find use in the present invention. Representative groups may be defined, for example, by similarity. Measures of similarity include, but are not limited to sequence similarity and energetic similarity. Thus the output sequences from computational screening may be clustered around local minima, referred to herein as clustered sets of sequences. For example, sets of sequences that are close in sequence space may be distinguished from other sets. In one embodiment, coverage within one or a subset of clustered sets may be maximized by including in the experimental library some, most, or all of the sequences that make up one or more clustered sets of sequences. For example, the user may wish to maximize coverage within the one, two, or three lowest energy clustered sets by including the majority of sequences within these sets in the library. In an alternate embodiment, diversity across clustered sets of sequences may be sampled by including within an experimental library only a subset of sequences within each clustered set. For example, all or most of the clustered sets could be broadly sampled by including the lowest energy sequence from each clustered set in the experimental library.

In some embodiments, sequences that do not make the cutoff are included in the experimental library. This may be desirable in some situations, for instance to evaluate the approach to library generation, to provide controls or comparisons, or to sample additional sequence space. For example, the WT antibody sequence may be included in the library, even if it does not make the cutoff.

The set of antibody sequences in an experimental library is generally, but not always, significantly different from the wild type antibody template, although in some cases the library preferably contains the wild-type sequence. The range of optimized protein sequences is dependent upon many factors including the size of the protein, properties desired, etc.

Use of Sequence Information to Guide Library Generation

In one embodiment of the present invention, sequence information may be used to guide or filter a computationally screened output for generation of an experimental library. As discussed, by comparing and contrasting alignments of antibody sequences, the degree of variability at a position and the types of amino acids which occur naturally at that position may be observed. Data obtained from such analyses are useful in the present invention. The benefits of using sequence information have been discussed, and those benefits apply equally to use of sequence information to guide library generation. The set of amino acids which occur in an antibody sequence alignment may be thought of as being pre-screened by evolution to have a higher chance than random at being compatible with an antibody's structure, stability, solubility, function, etc. Furthermore, certain alignments may provide represent sequences that are less immunogenic than random sequences. The variety of sequence sources, as well as the methods for generating antibody sequence alignments that have been discussed find use in the application of sequence information to guiding library generation. Likewise, as discussed above, various criteria may be applied to determine the importance or weight of certain residues in an alignment. These methods also find use in the application of sequence information to guide library generation.

Using sequence information to guide library generation from the results of computational screening finds broad use in the present invention. In one embodiment, sequence information is used to filter sequences from computational screening output. That is to say, some substitutions are subtracted from the computational output to generate the experimental library. For example, to optimize antibody solubility by replacing exposed nonpolar surface residues, the resulting output of a computational screening calculation or calculations may be filtered so that the experimental library includes only those amino acids, or a subset of those amino acids which meet some criteria, that are observed at that position in an alignment of antibody sequences. In an alternate embodiment, sequence information is used to add sequences to the computational screening output. That is to say, sequence information is used to guide the choice of additional amino acids that are added to the computational output to generate the experimental library. For example, to optimize antibody stability by mutating domain interface residues, the output set of amino acids for a given position from a computational screening calculation may be augmented to include one or more amino acids that are observed at that position in an alignment of antibody sequences. In an alternate embodiment, based on sequence alignment information, one or more amino acids may be added to or subtracted from the computational screening sequence output in order to maximize coverage or diversity. For example, additional amino acids with properties similar to those that are found in a sequence alignment may be added to the experimental library. For example, if a position involved in antigen binding is observed to have uncharged polar amino acids in an antibody sequence alignment, the user may choose to include additional uncharged polar amino acids to the experimental library at that position.

Generation of Secondary Libraries

In one embodiment of the present invention, libraries may be processed further to generate subsequent libraries. In this way, the output from a computational screening calculation or calculations may be thought of as a primary library. This primary library may be combined with other primary libraries from other calculations or other experimental libraries, processed using subsequent calculations, sequence information, or other analyses, or processed experimentally to generate a subsequent library, herein referred to as a secondary library, which could become an experimental library. As will be appreciated from this description, the use of sequence information to guide or filter libraries, discussed above, is itself one method of generating secondary libraries from primary libraries. Generation of secondary libraries gives the user greater control of the parameters within an experimental library. This enables more efficient experimental screening, and may allow feedback from experimental results to be interpreted more easily, providing a more efficient design/experimentation cycle.

There are a wide variety of methods to generate secondary libraries from primary libraries. For example, U.S. Ser. No. 10/218,102 and PCT 02/25588, herein expressly incorporated by reference, describes methods for secondary library generation that find use in the present invention. Typically some selection step occurs in which a primary library is processed in some way. For example, in one embodiment a selection step occurs where some set of primary sequences are chosen to form the secondary library. In an alternate embodiment, a selection step is a computational step, again generally including a selection step, wherein some subset of the primary library is chosen and then subjected to further computational analysis, including both further computational screening as well as techniques such as “in silico” shuffling (recombination). See, for example U.S. Pat. Nos. 5,830,721; 5,811,238; 5,605,793; 5,837,458, PCT US/19256, Rachitt-Enchira (.enchira.com/gene_shuffling.htm); error-prone PCR, for example using modified nucleotides; known mutagenesis techniques including the use of multi-cassettes; DNA shuffling (Crameri et al., 1998, Nature 391:288-291); heterogeneous DNA samples (U.S. Pat. No. 5,939,250); ITCHY (Ostermeier et al., 1999, Nat. Biotechnol. 17:1205-1209); StEP (Zhao et al., 1998, Nat. Biotechnol. 16:258-261), GSSM (U.S. Pat. No. 6,171,820 and U.S. Pat. No. 5,965,408); in vivo homologous recombination, ligase assisted gene assembly, end-complementary PCR, profusion (Roberts & Szostak, 1997, Proc. Natl. Acad. Sci. USA 94:12297-12302); yeast/bacteria surface display (Lu et al., 1995, Biotechnology 13:366-372); Seed & Aruffo, 1987, Proc. Natl. Acad. Sci. USA 84(10):3365-3369; Boder & Wittrup, 1997, Nat. Biotechnol. 15:553-557). all hereby incorporated by reference. In an alternate embodiment, a selection step occurs that is an experimental step, for example any of the experimental library screening steps below, wherein some subset of the primary library is chosen and then recombined experimentally, for example using one of the directed evolution methods discussed below, to form a secondary library. In a preferred embodiment, the primary library is generated and processed as outlined in U.S. Pat. No. 6,403,312, which is herein expressly incorporated by reference.

Generation of secondary and subsequent libraries finds broad use in the present invention. In one embodiment, different primary libraries may be combined to generate a secondary or subsequent library. In another embodiment, secondary libraries may be generated by sampling sequence diversity at highly mutatable or highly conserved positions. The primary library may be analyzed to determine which amino acid positions in the template protein have high mutational frequency, and which positions have low mutational frequency. For example, positions in an antibody that show a great deal of mutational diversity in computational screening may be fixed in a subsequent round of design calculations. A filtered set of the same size as the first would now show diversity at positions that were largely conserved in the first library. Alternatively, the secondary library may be generated by varying the amino acids at the positions that have high numbers of mutations, while keeping constant the positions that do not have mutations above a certain frequency.

This discussion is not meant to constrain generation of libraries subsequent to primary libraries to secondary libraries. As will be appreciated, primary and secondary libraries may be processed further to generate tertiary libraries, quaternary libraries, and so on. In this way, library generation is an iterative process. For example, tertiary libraries may be constructed using a variety of additional steps applied to one or more secondary libraries; for example, further computational processing may occur, secondary libraries may be recombined, or subsets of different secondary libraries may be combined. In a preferred embodiment, a tertiary library may be generated by combining secondary libraries. For example, primary and/or secondary libraries that analyzed different parts of a protein may be combined to generate a tertiary library that treats the combined parts of the protein. In an alternate embodiment, the variants from a primary library may be combined with the variants from a second library to provide a combined tertiary library at lower computational cost than creating a very long filtered set. These combinations may be used, for example, to analyze large proteins, especially large multi-domain proteins. Thus the above description of secondary library generation applies to generating any library subsequent to a primary library, the end result being a final library that may screened experimentally to obtain optimized antibodies. These examples are not meant to constrain generation of secondary libraries to any particular application or theory of operation for the present invention. Rather, these examples are meant to illustrate that generation of secondary libraries, and subsequent libraries such as tertiary libraries and so on, is broadly useful in computational screening methodology for experimental library generation.

Experimental Library Screening

Once an experimental library is designed using any of the methods outlined herein or combinations thereof, the physical library may be constructed using a variety of techniques. The library may then be screened to obtain antibodies optimized for greater stability, solubility, and/or enhanced affinity for antigen. Accordingly, the present invention provides a variety of methods for constructing and screening experimental libraries. These methods are not meant to constrain the present invention to any particular application or theory of operation. Rather, the provided examples are meant to illustrate generally that computationally screened libraries may be screened experimentally to obtain antibodies with optimized physico-chemical properties. General methods for antibody molecular biology, expression, purification, and screening are described in Antibody Engineering, 2001, edited by Duebel & Kontermann, Springer-Verlag, Heidelberg; Hayhurst & Georgiou, 2001, Curr. Opin. Chem. Biol. 5:683-689; Maynard & Georgiou, 2000, Annu. Rev. Biomed. Eng. 2:339-76; all of which are herein expressly incorporated by reference.

Molecular Biology and Library Generation

In one embodiment of the present invention, the experimental library sequences are used to create nucleic acids such as DNA which encode the antibody member sequences and which may then be cloned into host cells, expressed and assayed, if desired. Thus, nucleic acids, and particularly DNA, may be made which encode each member protein sequence. These practices are carried out using well-known procedures. For example, a variety of methods that may find use in the present invention are described in Molecular Cloning-A Laboratory Manual, 3^{rd} Ed. (Maniatis, Cold Spring Harbor Laboratory Press, New York, 2001), and Current Protocols in Molecular Biology (Wiley & Sons, mrw2.interscience.wiley.com/cponline/), both of which are herein expressly incorporated by reference.

As will be appreciated by those in the art, the generation of exact sequences for a library comprising a large number of sequences is potentially expensive and time consuming. Accordingly, there are a variety of techniques that may be used to efficiently generate experimental libraries of the present invention. Such methods that may find use in the present invention are described or referenced in U.S. Pat. No. 6,403,312; U.S. Ser. Nos. 09/782,004; 09/927,790 and 10/218,102; and PCTs 01/40091 and 02/25588, all hereby incorporated by reference. Such methods include but are not limited to gene assembly methods, PCR-based method and methods which use variations of PCR, ligase chain reaction-based methods, pooled oligo methods such as those used in synthetic shuffling, error-prone amplification methods and methods which use oligos with random mutations, classical site-directed mutagenesis methods, cassette mutagenesis, and other amplification and gene synthesis methods. As is known in the art, there are a variety of commercially available kits and methods for gene assembly, mutagenesis, vector subcloning, and the like, and such commercial products find use in the present invention for generating nucleic acids that encode members of an experimental library.

Protein Expression

Expression Systems

The library antibody proteins of the present invention may be produced by culturing a host cell transformed with nucleic acid, preferably an expression vector, containing nucleic acid encoding an library protein, under the appropriate conditions to induce or cause expression of the library protein. The conditions appropriate for library protein expression will vary with the choice of the expression vector and the host cell, and will be easily ascertained by one skilled in the art through routine experimentation.

A wide variety of appropriate host cells may be used, including but not limited to mammalian cells, bacteria, insect cells, and yeast. For example, a variety of cell lines that may find use in the present invention are described in the ATCC cell line catalog (atcc.org), herein expressly incorporated by reference.

In a preferred embodiment, the library proteins are expressed in mammalian expression systems, including systems in which the expression constructs are introduced into the mammalian cells using virus such as retrovirus or adenovirus. Any mammalian cells may be used, with mouse, rat, primate and human cells being particularly preferred. Suitable cells also include known research cells, including but not limited to Jurkat T cells, NIH3T3 cells, CHO, COS, etc. In an alternately preferred embodiment, library proteins are expressed in bacterial systems. Bacterial expression systems are well known in the art, and include Escherichia coli (E. coli), Bacillus subtilis, Streptococcus cremoris, and Streptococcus lividans. In an alternate embodiment, library proteins are produced in insect cells. In an alternate embodiment, library proteins are produced in yeast cells. In an alternate embodiment library proteins are expressed in vitro using cell free translation systems. In vitro translation systems derived from both prokaryotic (e.g. E. coli) and eukaryotic (e.g. wheat germ, rabbit reticulocytes) cells are available and may be chosen based on the expression levels and functional properties of the protein of interest. For example, as appreciated by those skilled in the art, in vitro translation is required for some display technologies, for example ribosome display. In addition, the library proteins may be produced by chemical synthesis methods.

Expression Vectors

The nucleic acids that encode the antibody library members may be incorporated into an expression vector in order to express the protein. A variety of expression vectors may be utilized to express the library proteins. Expression vectors may comprise self-replicating extra-chromosomal vectors or vectors which integrate into a host genome. Expression vectors are constructed to be compatible with the host cell type. Thus expression vectors which find use in the present invention include but are not limited to those which enable protein expression in mammalian cells, bacteria, insect cells, and yeast. As is known in the art, a variety of expression vectors are available, commercially or otherwise, that may find use in the present invention for expressing antibody library proteins.

Expression vectors typically comprise a library member operably linked with control or regulatory sequences, selectable markers, any fusion partners, and/or additional elements. By “operably linked” herein is meant that the nucleic acid is placed into a functional relationship with another nucleic acid sequence. Generally, these expression vectors include transcriptional and translational regulatory nucleic acid operably linked to the nucleic acid encoding the library antibody, and are typically appropriate to the host cell used to express the library protein. In general, the transcriptional and translational regulatory sequences may include, but are not limited to, promoter sequences, ribosomal binding sites, transcriptional start and stop sequences, translational start and stop sequences, and enhancer or activator sequences. As is also known in the art, expression vectors typically contain a selection gene or marker to allow the selection of transformed host cells containing the expression vector. Selection genes are well known in the art and will vary with the host cell used.

Fusion Partners

Antibody library members may be operably linked to a fusion partner to enable targeting of the expressed protein, purification, screening, display, and the like. Fusion partners may be linked to the library member sequence via a linker sequences. The linker sequence will generally comprise a small number of amino acids, typically less than ten, although longer linkers may also be used. Typically, linker sequences are selected to be flexible and resistant to degradation. As will be appreciated by those skilled in the art, any of a wide variety of sequences may be used as linkers. For example, a common linker sequence comprises the amino acid sequence GGGGS.

A fusion partner may be a targeting or signal sequence that directs library antibody protein and any associated fusion partners to a desired cellular location or to the extracellular media. As is known in the art, certain signaling sequences may target a protein to be either secreted into the growth media, or into the periplasmic space, located between the inner and outer membrane of the cell.

A fusion partner may also be a sequence that encodes a peptide or protein that enables purification and/or screening. Such fusion partners include but are not limited to polyhistidine tags (for example His₆ and His₁₀ or other tags for use with Immobilized Metal Affinity Chromatography (IMAC) systems (e.g. Ni⁺² affinity columns)), GST fusions, MBP fusions, Strep-tag, the BSP biotinylation target sequence of the bacterial enzyme BirA, and epitope tags which are targeted by antibodies (for example to c-myc tags, flag tags, and the like). As will be appreciated by those skilled in the art, such tags may be useful for purification, for screening, or both. For example, an antibody fragment may be purified using a His-tag by immobilizing it to a Ni⁺² affinity column, and then after purification the same His-tag may be used to immobilize the antibody to a Ni⁺² coated plate to perform an ELISA or other binding assay (see “Screening of Library Members” section below).

A fusion partner may enable the use of a selection method to screen antibody library members (see “Screening based on selection methods” below). Fusion partners which enable a variety of selection methods are well-known in the art, and all of these find use in the present invention. For example, by fusing the members of an antibody library to the gene III protein, phage display can be used (Kay et al., 1996, Phage display of peptides and proteins: a laboratory manual, Academic Press, San Diego, Calif.); Lowman et al., 1991, Biochemistry 30:10832-10838; Smith, 1985, Science 228:1315-1317). Fusion partners may enable antibody library members to be labeled. Alternatively, a fusion partner may bind to a specific sequence on the expression vector, enabling the fusion partner and associated antibody library member to be linked covalently or noncovalently with the nucleic acid that encodes them. For example, U.S. Ser. Nos. 09/642,574; 10/080,376; 09/792,630; 10/023,208; 09/792,626; 10/082,671; 09/953,351; 10/097,100; and 60/366,658; PCTs 00/22906; 01/49058; 02/04852; 02/04853; 02/08023; 01/28702; and 02/07466; all herein expressly incorporated by reference, describe such a fusion partner and technique that may find use in the present invention.

Transformation and Transfection Methods

The methods of introducing exogenous nucleic acid into host cells is well known in the art, and will vary with the host cell used. Techniques include but are not limited to dextran-mediated transfection, calcium phosphate precipitation, calcium chloride treatment, polybrene mediated transfection, protoplast fusion, electroporation, viral or phage infection, encapsulation of the polynucleotide(s) in liposomes, and direct microinjection of the DNA into nuclei. In the case of mammalian cells, transfection may be either transient or stable.

Protein Purification

In a preferred embodiment, antibody library members are purified or isolated after expression. Antibodies may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind antibodies, and these proteins can find use in the present invention for purification of antibody library members. For example, the bacterial proteins A and G bind to the Fc region, and the bacterial protein L binds to the Fab region. Purification can often be enabled by a particular fusion partner. For example, antibody library members may be purified using glutathione resin if a GST fusion is employed, Ni⁺² affinity chromatography if a His tag is employed, or immobilized anti-flag antibody if a flag tag is used. For general guidance in suitable purification techniques, see Protein Purification: Principles and Practice, 3^{rd} Ed., Scopes, Springer-Verlag, N.Y., 1994, hereby expressly incorporated by reference.

The degree of purification necessary will vary depending on the screen or use of the antibody library members. In some instances no purification is necessary. For example in one embodiment, if library antibodies are secreted, screening may take place directly from the media. As is well known in the art, some methods of selection do not involve purification of library proteins. Thus, for example, if the optimized antibody sequences are made into a phage display library, antibody purification may not be performed.

Screening of Library Members

Library members may be screened using a variety of methods, including but not limited to those that use in vitro assays, in vivo and cell-based assays, and selection technologies. Automation and high-throughput screening technologies may be utilized in the screening procedures. Screening may employ the use of a fusion partner or label. The use of fusion partners has been discussed above. By “labeled” herein is meant that the antibodies of the invention have one or more elements, isotopes, or chemical compounds attached to enable the detection in a screen. In general, labels fall into three classes: a) immune labels, which may be an epitope incorporated as a fusion partner that is recognized by an antibody, b) isotopic labels, which may be radioactive or heavy isotopes, and c) small molecule labels, which may include fluorescent and colorimetric dyes, or molecules such as biotin which enable other labeling methods. Labels may be incorporated into the compound at any position and may be incorporated in vitro or in vivo during antibody expression.

In vitro Assays

In a preferred embodiment, the functional and/or biophysical properties of antibody library members are screened in an in vitro assay. In vitro assays may allow a broad dynamic range for screening antibody properties of interest. Properties of library members that may be screened include but are not limited to stability, solubility, and affinity for antigen, antibody receptors, or other proteins which are known to bind the antibody being optimized. Multiple properties may be screened simultaneously or individually. Proteins may be purified or unpurified, depending on the requirements of the assay.

In one embodiment, the screen is a qualitative or quantitative binding assay for binding of antibody library members to a protein or nonprotein molecule that is known to bind the antibody. In a preferred embodiment, the screen is a binding assay for measuring the binding of antibody library members to the antibody's antigen. In an alternately preferred embodiment, the screen is an assay for antibody binding to an antibody receptor or some other protein that is known to bind antibodies. For example, a number of proteins are known to bind the Fc region (Ravetch & Bolland, 2001, Ann. Rev. Immunol. 19:275-90; Raghavan & Bjorkman, 1996, Annu. Rev. Cell Dev. Biol. 12:181-220), including the family of FcγRs, the neonatal receptor FcRn, the complement protein C1q, and the bacterial proteins A and G. Binding assays can be carried out using a variety of methods known in the art. These methods include but are not limited to FRET (Fluorescence Resonance Energy Transfer) and BRET (Bioluminescence Resonance Energy Transfer)-based assays, AlphaScreen (Amplified Luminescent Proximity Homogeneous Assay), Scintillation Proximity Assay, ELISA (Enzyme-Linked Immunosorbent Assay), SPR (Surface Plasmon Resonance) or BIACORE, isothermal titration calorimetry, differential scanning calorimetry, gel electrophoresis, and chromatography including gel filtration. These and other methods may take advantage of some fusion partner or label of the antibody library member. Assays may employ a variety of detection methods including but not limited to chromogenic, fluorescent, luminescent, or isotopic labels.

The biophysical properties of antibodies, for example stability and solubility, may be screened using a variety of methods known in the art. Protein stability may be determined by measuring the thermodynamic equilibrium between folded and unfolded states. For example, antibody library members of the present invention may be unfolded using chemical denaturant, heat, or pH, and this transition may be monitored using methods including but not limited to circular dichroism spectroscopy, fluorescence spectroscopy, absorbance spectroscopy, NMR spectroscopy, calorimetry, and proteolysis. As will be appreciated by those skilled in the art, the kinetic parameters of the folding and unfolding transitions may also be monitored using these and other techniques. The solubility and overall structural integrity of an antibody may be quantitatively or qualitatively determined using a wide range of methods that are known in the art. Methods which may find use in the present invention for characterizing the biophysical properties of antibody library members include gel electrophoresis, chromatography such as size exclusion chromatography and reversed-phase high performance liquid chromatography, mass spectrometry, ultraviolet absorbance spectroscopy, fluorescence spectroscopy, circular dichroism spectroscopy, isothermal titration calorimetry, differential scanning calorimetry, analytical ultra-centrifugation, dynamic light scattering, proteolysis, and cross-linking, turbidity measurement, filter retardation assays, immunological assays, fluorescent dye binding assays, protein-staining assays, microscopy, and detection of aggregates via ELISA. Structural analysis employing X-ray crystallographic techniques and NMR spectroscopy may also find use. In one embodiment, antibody stability and/or solubility may be measured by determining the amount of antibody in solution after some defined period of time. In this assay, the antibody may or may not be exposed to some extreme condition, for example elevated temperature, low pH, or the presence of denaturant. Because antibody function typically requires a stable, soluble, and/or well-folded/structured antibody, the functional (i.e. binding) assays described above also provide a way to perform such an assay. For example, a solution comprising an antibody variant could be assayed for its ability to bind antigen, then exposed to elevated temperature for one or more defined periods of time, then assayed for antigen binding again. Because unfolded and aggregated antibody is not expected to be capable of binding antigen, the amount of antibody activity remaining provides a measure of the antibody variant's stability and solubility.

In Vivo or Cell-based Assays

In a preferred embodiment, the library is screened using one or more cell-based or in vivo-based assays. Cell types for such assays may be prokaryotic or eukaryotic. For such assays, antibody library members, purified or unpurified, are typically added exogenously such that cells are exposed to individual variants or pools of variants belonging to a library. These assays are typically, but not always, based on the function of the antibody, that is the ability of the antibody to bind an antigen and/or some protein which naturally binds the antibody, for example an Fc receptor. Such assays often involve monitoring the response of cells to antibody, for example cell survival, cell death, change in cellular morphology, or transcriptional activation such as cellular expression of a natural gene or reporter gene. For example, anti-cancer antibodies may cause apoptosis of certain cell lines expressing the antibody's target antigen, or they may mediate attack on target cells by immune cells which have been added to the assay. Methods for monitoring cell death or viability are known in the art, and include the use of dyes, immunochemical, cytochemical, or radioactive reagents. For example, caspase staining assays may enable apoptosis to be measured, and uptake of radioactive substrates or the dye alamar blue may enable cell growth or activation to be monitored. Transcriptional activation may also serve as a method for assaying antibody function in cell-based assays. In this case, response may be monitored by assaying for natural genes or proteins which may be upregulated, for example the release of certain interleukins may be measured, or alternatively readout may be via a reporter construct. Cell-based assays may also involve the measure of morphological changes of cells as a response to the presence of an antibody library variant.

Alternatively, cell-based screens are performed directly using cells that have been transformed or transfected with nucleic acids encoding antibody library members. That is, antibody library variants are not added exogenously to the cells. For example, in one embodiment, the cell-based screen utilizes cell surface display. A fusion partner can be employed that enables display of antibodies on the surface of cells (Witrrup, 2001, Curr. Opin. Biotechnol., 12:395-399). Cell surface display methods which may find use in the present invention include but are not limited to display on bacteria (Georgiou et al., 1997, Nat Biotechnol. 15:29-34.; Georgiou et al., 1993, Trends Biotechnol. 11:6-10; Lee et al., 2000, Nat. Biotechnol. 18:645-648; Jun et al, 1998, Nat. Biotechnol. 16:576-80.), yeast (Boder & Wittrup, 2000, Methods Enzymol. 328:430-44; Boder & Wittrup, 1997, Nat. Biotechnol. 15:553-557), and mammalian cells (Whitehorn et al, 1995, Biotechnology 13:1215-1219). In an alternate embodiment, antibodies are not displayed on the surface of cells, but rather are screened intracellularly or in some other cellular compartment. For example, periplasmic expression and cytometric screening (Chen et al, 2001, Nat. Biotechnol., 19:537-542), the protein fragment complementation assay (Johnsson & Varshavsky, 1994, Proc. Natl. Acad. Sci. USA, 91:10340-10344.; Pelletier et al., 1998, Proc. Natl. Acad. Sci. USA 95:12141-12146), and the yeast two hybrid screen (Fields & Song, 1989, Nature 340:245-246) may find use in the present invention.

Alternatively, if the antibody imparts some selectable growth advantage to a cell, this property may be used to screen or select for antibody variants.

The biological properties of one or more antibody library members, including clinical efficacy, pharmacokinetics, and toxicity, may also be characterized in cell, tissue, and whole organism experiments.

Screening Based on Selection Methods

As is known in the art, a subset of screening methods are those that select for favorable members of a library. Said methods are herein referred to as “selection methods”, and these methods find use in the present invention for screening antibody libraries. When antibody libraries are screened using a selection method, only those members of a library which are favorable, that is which meet some selection criteria, are propagated, isolated, and/or observed. As will be appreciated, because only the most fit antibody variants are observed, such methods enable the screening of libraries which are larger than those screenable by methods which assay the fitness of library members individually. Selection is enabled by any method, technique, or fusion partner which links, covalently or noncovalently, the phenotype of an antibody variant with its genotype, that is the function of an antibody with the nucleic acid that encodes it. For example the use of phage display as a selection method is enabled by the fusion of library members to the gene III protein. In this way, selection or isolation of antibody proteins which meet some criteria, for example binding affinity for antigen, also selects for or isolates the nucleic acid which encodes it. Once isolated, the gene or genes encoding library antibody variants may then be amplified. This process of isolation and amplification, referred to as panning, may be repeated, allowing favorable antibody variants in the library to be enriched. Nucleic acid sequencing of the attached nucleic acid ultimately allows for gene identification.

A variety of selection methods are known in the art which may find use in the present invention for screening antibody libraries. These include but are not limited to phage display (Phage display of peptides and proteins: a laboratory manual, Kay et al., 1996, Academic Press, San Diego, Calif.; Lowman et al., 1991, Biochemistry 30:10832-10838; Smith, 1985, Science 228:1315-1317) and its derivatives such as selective phage infection (Malmborg et al., 1997, J. Mol. Biol. 273:544-551), selectively infective phage (Krebber et al., 1997, J. Mol. Biol. 268:619-630), and delayed infectivity panning (Benhar et al., 2000, J. Mol. Biol. 301:893-904), cell surface display (Witrrup, 2001, Curr. Opin. Biotechnol., 12:395-399) such as display on bacteria (Georgiou et al., 1997, Nat. Biotechnol. 15:29-34.; Georgiou et al., 1993, Trends Biotechnol. 11:6-10; Lee et al., 2000, Nat. Biotechnol. 18:645-648; Jun et al., 1998, Nat. Biotechnol. 16:576-80), yeast (Boder & Wittrup, 2000, Methods Enzymol. 328:430-44; Boder & Wittrup, 1997, Nat. Biotechnol. 15:553-557), and mammalian cells (Whitehorn et al., 1995, Bioltechnology 13:1215-1219), as well as in vitro display technologies (Amstutz et al., 2001, Curr. Opin. Biotechnol. 12:400-405) such as polysome display (Mattheakis et al., 1994, Proc. Natl. Acad. Sci. USA 91:9022-9026), ribosome display (Hanes et al, 1997, Proc. Natl. Acad. Sci. USA 94:4937-4942), mRNA display (Roberts & Szostak, 1997, Proc. Natl. Acad. Sci. USA 94:12297-12302; Nemoto et al., 1997, FEBS Lett. 414:405-408), and ribosome-inactivation display system (Zhou et al., 2002, J. Am. Chem. Soc. 124, 538-543)

Other selection methods which may find use in the present invention include methods that do not rely on display, such as in vivo methods including but not limited to periplasmic expression and cytometric screening (Chen et al, 2001, Nat. Biotechnol., 19:537-542), the protein fragment complementation assay (Johnsson & Varshavsky, 1994, Proc. Natl. Acad. Sci. USA, 91:10340-10344; Pelletier et al., 1998, Proc. Natl. Acad. Sci. USA 95:12141-12146), and the yeast two hybrid screen (Fields & Song, 1989, Nature 340:245-246) used in selection mode (Visintin et al., 1999, Proc. Natl. Acad. Sci. USA 96: 11723-11728). In an alternate embodiment, selection is enabled by a fusion partner which binds to a specific sequence on the expression vector, thus linking covalently or noncovalently the fusion partner and associated antibody library member with the nucleic acid that encodes them. In an alternative embodiment, in vivo selection can occur if expression of the library antibody imparts some growth, reproduction, or survival advantage to the cell.

As is known in the art, a subset of selection methods referred to as “directed evolution methods” are those that include the mating or breading of favorable sequences during selection, sometimes with the incorporation of new mutations. As will be appreciated by those skilled in the art, directed evolution methods can facilitate identification of the most favorable sequences in a library, and can increase the diversity of sequences that are screened. A variety of directed evolution methods are known in the art that may find use in the present invention for screening antibody libraries, including but not limited to DNA shuffling (WO 00/42561 A3; WO 01/70947 A3), exon shuffling (U.S. Pat. No. 6,365,377 B1; Kolkman & Stemmer, 2001, Nat. Biotechnol. 19:423-428), family shuffling (Crameri et al., 1998, Nature 391:288-291; U.S. Pat. No. 6,376,246 B1), RACHIT™ (Coco et al., 2001, Nat. Biotechnol. 19:354-359; WO 02/06469 A2), STEP and random priming of in vitro recombination (Zhao et al., 1998, Nat. Biotechnol. 16:258-261; Shao et al., 1998, Nucleic Acids Res. 26:681-683), exonuclease mediated gene assembly (U.S. Pat. No. 6,352,842 B1; U.S. Pat. No. 6,361,974 B1), Gene Site Saturation Mutagenesis™ (U.S. Pat. No. 6,358,709 B1), Gene Reassembly™ (U.S. Pat. No. 6,358,709B1), SCRATCHY (Lutz et al., 2001, Proc. Natl. Acad. Sci. USA 98:11248-11253), DNA fragmentation methods (Kikuchi et al., Gene 236:159-167), and single-stranded DNA shuffling (Kikuchi et al., 2000, Gene 243:133-137), all of which are herein expressly incorporated by reference.

Design Strategies

A variety of computational screening design strategies are provided for optimization of the physico-chemical properties of antibodies, including stability, solubility, and antigen binding affinity. These strategies can be used individually or in combination.

Stability Optimization

There is frequently a need to enhance the stability of an antibody. Lower stability of a full-length antibody or an antibody fragment may result in greater amount of nonnative and thus nonfunctional species, increased susceptibility to degradation, and greater tendency for aggregation. Increased degradation and aggregation may result in lower in vivo half-life of the molecule if the antibody is a therapeutic, further decreasing activity.

In one object of the present invention, computational screening methodology is used to enhance the stability of an antibody. A number of design strategies are disclosed for antibody stabilization, including strategies which employ experimental information and/or sequence information to guide choice of variable positions, choice of amino acids considered at those positions, and/or generation of one or more experimental libraries from computational output. The disclosed design strategies are not meant to constrain the present invention to any particular application or theory of operation. Rather, the present invention relates as novel not only these provided individual strategies, but the general use of computational screening to enhance the stability of antibodies.

The stability of an antibody is comprised of: a) the stabilities of each individual Ig domain which make up the antibody, and b) the stabilities or affinities of interdomain interactions if the antibody is composed of more than one Ig domain. Thus two main strategies for utilizing computational screening methodology to stabilize antibodies are to enhance the stability of individual Ig domains, and enhance interface stability between individual Ig domains.

Domain Stability

The stability of an antibody is determined in part by the individual stabilities of each of the Ig domains that comprise it. In one embodiment, computational screening is used to stabilize an antibody by enhancing the stability of one or more individual Ig domains. In this embodiment, more favorable interactions are designed within one or more individual Ig domains, thereby increasing the global stability of the antibody as a whole. For an antibody which is made up of more than one Ig domain, each individual Ig domain may be engineered for greater stability. Thus for example, for antibodies derived from human, mouse, rat, or rabbit antibodies, the stability may be improved by stabilizing one or more of domains V_{H,} V_{L,} Cγ1, C_{L}, Cγ2, and Cγ3.

In one embodiment, the interior of an Ig domain or Ig domains are redesigned to be more stable. For example, as will be appreciated by those skilled in the art, the van der Waals packing interactions between nonpolar residues in the core play an important role protein stability. Mutations may be designed that result in more favorable interactions between interior residues. In another embodiment, non-interior residues, that is boundary or surface positions an Ig domain or domains are designed to be more stable. For example, greater stability may be gained when amino acid side chains which have the capacity to donate a hydrogen bond are interacting with a molecule which is capable of accepting a hydrogen bond, whether this molecule be another side chain, the protein backbone, or solvent. Interior and non-interior residues may be identified by objective methods such as degree of solvent exposure, as described above, subjective methods such as visual inspection by one skilled in the art of protein structural biology, or other methods. As described above, variable positions and amino acids considered at those positions may be chosen using any variety of approaches, including but not limited to approaches based on solvent exposure, approaches which are hypothesis-driven, approaches which utilize experimental information, approaches which utilize sequence information, or any combination of these and other approaches.

A number of examples are provided below which describe the use of computational screening methods to stabilize the Ig domains of an antibody. These examples are not meant to constrain the present invention to any particular application or theory of operation. Rather, the present invention relates as novel not only these provided individual examples, but the general use of computational screening methodology to enhance the stability of an Ig domain or Ig domains in order to optimize an antibody for greater stability.

Interface Stability

The stability of multi-Ig domain antibodies, that is to say full-length antibodies and antibody fragments which are composed of more than one Ig domain, are determined in part by the affinities of the interactions between domains (Worn & Plückthun, 2001, J. Mol. Biol. 305:989-1010). Two interacting Ig domains exist in equilibrium between bound and unbound states. In the unbound state, Ig domains have a greater tendency to unfold and aggregate than when they are in the bound state. Thus by designing more favorable interactions between residues that mediate the interdomain interaction, the bound state may be stabilized, thereby stabilizing the antibody as a whole. In one embodiment of the present invention, computational screening is used to engineer mutations that result in more favorable interactions between individual Ig domains. As shown in FIG. 1, for human antibodies there are five interdomain interfaces that may be optimized using computational screening methodology: V_{H}-V_{L}, Cγ1-C_{L}, V_{H}-CΔ1, V_{L}-C_{L}, and Cγ3-Cγ3. The stability of a Fab is dependent on the interactions at only a subset of these interfaces: V_{H}-V_{L}, Cγ1-C_{L}, V_{H}-Cγ1, and V_{L}-C_{L}.

Greater interdomain stability may be obtained by engineering more energetically favorable interactions between residues that mediate the interdomain interface. Such designed interactions could involve more favorable packing interactions, hydrogen bond interactions, electrostatic interactions, hydrophobic interactions, and the like. Interface residues may be identified by objective methods such as degree of solvent exposure, as described above, subjective methods such as visual inspection by one skilled in the art of protein structural biology, or other methods. As described above, variable positions and amino acids considered at those positions may be chosen using any variety of approaches, including but not limited to approaches based on solvent exposure, approaches which are hypothesis-driven, approaches which utilize experimental information, approaches which utilize sequence information, or any combination of these and other approaches.

In one embodiment, the interface is designed to have more favorable nonpolar interactions, for example by engineering the interface with more nonpolar volume than that in the antibody template, by designing nonpolar residues which pack better together than that in the antibody template, and the like. As will be appreciated by those skilled in the art, this may be thought of as the interface version of a redesigned hydrophobic core. Here, however, variable positions are those that make up the interface between Ig domains instead of the core of an Ig domain. In an alternate embodiment, the interface is designed to have more favorable polar interactions, for example by engineering the interface with more polar amino acids than that in the antibody template, by designing nonpolar residues with more optimized hydrogen bonds, electrostatic interactions, and the like. As will appreciated by those in the art, greater polar character at the interface may enable the bound/unbound equilibrium between Ig domains to be more reversible. In the unbound state, the residues which make up the interface with the other Ig domain and are normally sequestered from solvent become exposed to solvent. Nonpolar residues have a higher tendency to aggregate than polar residues, and therefore greater nonpolar character at the interdomain interface may result in a greater tendency to aggregate in the unbound form, resulting in non-reversibility of the unbinding/binding transition. Irreversible aggregation means that the antibody cannot get back to its native bound state (i.e. the Ig domain interface is not reformed). This property of Ig domain interfaces in antibodies is supported experimentally (Worn & Plückthun, 2001, J. Mol. Biol. 305:989-1010; Ewert et a., 2002, Biochemistry, 41:3628-3636). In an alternate embodiment, the interface is engineered with more favorable nonpolar and polar interactions.

A number of examples are provided below in which describe the use of computational screening methods to stabilize the interfaces between Ig domains. These examples illustrate how a variety of interactions may be designed at interdomain interfaces that result in greater stability. These examples are not meant to constrain the present invention to any particular application or theory of operation. Rather, the present invention relates as novel not only these provided individual examples, but the general use of computational screening methodology to design more energetically favorable inter-Ig domain interactions in order to stabilize an antibody.

Solubility Optimization

There is frequently a need to enhance the solubility of an antibody. Lower solubility of an antibody may result in a greater fraction of nonfunctional species, increased susceptibility to degradation, and shorter in vivo half-life and lower efficacy if the antibody is a therapeutic. Poor solubility may also place severe constraints on antibody formulation and route of administration. A number of design strategies are suggested for using computational screening methods to enhance the solubility of an antibody, all of which are embodiments of the present invention.

In one embodiment, surface exposed nonpolar residues in an antibody are replaced with polar residues which are predicted by computational screening calculations to be favorable. Underlying this strategy is the principle that polar residues are more soluble than nonpolar ones. This principle is well known in the art. In regard to which residues are more polar or nonpolar than others, such a judgment may be made subjectively or objectively. Subjectively, for example, one skilled in the art of protein structural biology appreciates qualitatively that amino acids such as leucine, tryptophan, and methionine are more nonpolar, and thus potentially more prone to cause aggregation when exposed to solvent, than amino acids such as serine, asparagine, and glutamate. Objective and quantitative measurements of hydrophobicity are also known in the art. For example, the free energies of transfer of an amino acid from non-aqueous to aqueous solution have been used to generate relative rankings of amino acid hydrophobicity, and such methods find use in the present invention. Variable positions and amino acids considered at those positions may be chosen using any variety of approaches, as described above, including but not limited to approaches based on solvent exposure, approaches which are hypothesis-driven, approaches which utilize experimental information, approaches which utilize sequence information, or any combination of these and other approaches.

A number of strategies for replacing exposed nonpolar amino acids find use in the present invention. In one embodiment, residues which may be replaced include residues which are exposed to solvent on individual Ig domains, or which lie at the interface between Ig domains. In this regard, all Ig domains of a human antibody, including V_{H}, V_{L}, Cγ1, C_{L}, Cγ2, and Cγ3, as well as the linkers and/or hinges which connect them, have surface residues which could be replaced with amino acids which may impart greater solubility to the antibody. In another embodiment, variable positions reside in a region of an antibody fragment which in the context of a full-length antibody or larger antibody fragment makes up the interface with another Ig domain. As will be appreciated by those skilled in the art, antibody fragments are generated by removing certain regions or domains of an antibody. As a result, regions of an Ig domain which interact with another Ig domain in the larger antibody may become exposed to solvent in the context of an antibody fragment. For example, the V_{H} and V_{L} residues which make up the V_{H}/Cγ1 and V_{L}/C_{L} interfaces of an antibody are exposed to solvent in an scFv fragment of that antibody (Nieba et al., 1997, Protein Eng. 10:435-44). The result for an scFv, or any other antibody fragment, may be increased propensity for aggregation and thus lower solubility. Computational screening methods may be used to engineer mutations at these positions which result in greater solubility of the antibody fragment.

Several additional strategies may also be used to optimize solubility. For example, it is known in the art that protein solubility is typically lowest when the pH of the solution is equal to the isoelectric point (pI) of the protein. Under such conditions, the net charge of the protein is equal to zero. It is possible to optimize solubility by altering the number and location of ionizable residues in the antibody to adjust the pI. In other cases, improvements in solubility may result from optimizing the stability of the antibody, as discussed above. As is well known in the art, proteins are much more prone to aggregation in unfolded or partially folded states. Thus proteins that are well folded, structured, and/or stable are typically more soluble. Accordingly, computational screening which stabilizes an antibody, for example by one or more design strategies discussed above, may also be used to enhance antibody solubility. Additionally, if the antibody contains one or more cysteines that do not form disulfide bonds in the native antibody structure, replacing such cysteines with less reactive, structurally compatible residues can prevent the formation of unwanted intra- and inter-molecular disulfide bonds. As will be appreciated by those skilled in the art, additional strategies could also be used to optimize the solubility of antibodies.

Affinity Maturation

There is frequently a need to enhance the affinity of an antibody for its antigen. This process is referred to as affinity maturation, and following this process, the antibody may then be said to be affinity matured. The binding affinity of an antibody for its target is a critical parameter for its success as a therapeutic, diagnostic, or reagent. Higher affinity for antigen may result in a more efficacious antibody therapeutic. As discussed above, enhancement of antigen affinity is frequently wanted or needed for a variety of forms and sources of antibodies such as those that are substantially human, nonhuman, chimeric, or humanized. A particular case which demands affinity maturation is subsequent to humanization. As discussed above, this technique to reduce the immunogenicity of antibody therapeutics often results in loss of binding affinity for antigen, and thus regaining this affinity is typically desired.

Computational screening methods may be applied to antibody affinity maturation using a number of design strategies, all of which are embodiments of the present invention. Strategies for affinity maturation include but are not limited to those which use only a structure or structures of bound antibody/antigen complexes, only a structure or structures of unbound antibodies, or structures of both bound and unbound antibody. These strategies need not be defined by the structural information that is available, but rather may be defined by the structural information that is employed. For example, to affinity mature an antibody it may be useful to carry out design calculations on an unbound antibody template that is a structure of the antibody alone without antigen, even though a structure of the antibody/antigen complex may be available. The structure of the unbound antibody may be available, or could be obtained by deleting antigen coordinates from the structure of the complex.

As discussed above, antibody templates may be obtained from a variety of sources, including but not limited to X-ray crystallographic techniques, NMR techniques, de novo modeling, and homology modeling. Antibody/antigen complexes may furthermore be obtained using docking methods. For example, if the antibody/antigen complex structure is not available, it may be modeled by docking the antigen into the antibody variable region. Methods for this process are known in the art. Variable positions and amino acids considered at those positions may be chosen using any variety of approaches, as described above, including but not limited to approaches based on solvent exposure, approaches which are hypothesis-driven, approaches which utilize experimental information, approaches which utilize sequence information, or any combination of these and/or other approaches.

In one embodiment, computational screening is used to affinity mature an antibody by using the structure of a bound antibody/antigen complex as the template for design calculations. In this strategy, one or more antibody mutations are design that result in more favorable interactions (i.e., higher affinity) between the antibody and its antigen. In one embodiment, only antibody residues which directly contact antigen, referred to herein as “contact residues” are allowed to vary in design calculations. In an alternate embodiment, variable antibody positions may include residues which do not contact antigen, alone or in addition to residues which do contact antigen. For example, the variable positions in a design calculation could be set to those residues which interact with contact residues, but are not themselves contact residues. As will be appreciated by those skilled in the art, the subtle conformations of contact residues which are optimal for antigen binding are determined in part by the conformations of the surrounding residues. By using computational screening to explore substitutions in the shell of residues which interact with contact residues, a quality diversity of new contact residue conformations may be sampled. In an alternate embodiment, contact residues and residues which are not contact residues are variable positions in design calculations.

In another embodiment, computational screening is used to affinity mature an antibody by using the structure of an uncomplexed antibody structure, i.e. a structure of an antibody which is not bound to its antigen, as the template for design calculations. In this strategy, antibody residues which contact antigen or which are believed to contact antigen are mutated to residues which are energetically favorable in the context of the structural template. The primary goal of this approach is to generate quality diversity within an experimental library such that the distribution within the library is skewed towards a larger percentage of variants which are energetically compatible with the antibody than would be expected if variants were designed randomly. Although the antibody variants in this library are not directly computationally screened to possess higher affinity for antigen, such variants will likely still be present in the library. The use of computational screening enables the vast sequence space of mutations which are inconsistent with the antibody structure to be trimmed from the library, thereby increasing the chances of finding in an experimental screen those variants which possess higher antigen binding affinity. In the absence of an antibody/antigen complex structure, it is not possible to identify contact residues by visual inspection. Thus, experimental and sequence information are particularly useful in this case, as these may provide insight into which residues are important determinants of antigen binding.

In another embodiment, computational screening methods are used to affinity mature an antibody by combining results from design calculations which use the structures of both a bound antibody/antigen complex and an unbound antibody structure as templates for design calculations. In one embodiment, computational screening is used to engineer mutations at or near the antibody/antigen interface that are energetically favorable in the context of both the bound and unbound antibody structures. For this strategy, output from two sets of design calculations could be used to generate an experimental library. For example, one set of calculations could involve those which use one or more unbound antibody structures as the template(s), and another set of calculations could use one or more bound antibody/antigen structures as the template(s). The experimental library could be comprised of variants which are predicted to be energetically favorable in both sets of calculations. In one embodiment, variants which are predicted to be energetically favorable in both structures are included in the library. In an alternate embodiment, variants which are predicted to be energetically favorable in at least one of the structures are included in the library. As is illustrated in the examples below, it is a preferred embodiment to have at least one of the variable regions located in a framework region, a complementarity determining region or a combination of both regions.

A number of examples are provided below which describe the use of computational screening to affinity mature antibodies. These examples are not meant to constrain the present invention to any particular application or theory of operation. Rather, the present invention relates as novel not only these provided individual examples, but the general use of computational screening methods to affinity mature antibodies.

### EXAMPLES

A number of examples are provided below to illustrate implementation of the design strategies discussed above to optimize antibodies. These examples employ a variety of strategies, approaches, methods, and so forth to choose variable positions, choose amino considered at those positions, calculate energies, search sequence space using optimization algorithms, and generate experimental libraries. Libraries generated from these examples could be subsequently screened experimentally to obtain optimized antibody variants, become part of other libraries which could be subsequently screened experimentally, or serve other purposes. These examples are not meant to constrain the present invention to any particular application or theory of operation. Rather, the present invention relates as novel not only to these provided individual examples, but the general use of computational screening to enhance antibody stability, improve antibody solubility, and increase the affinity of antibodies for antigen.

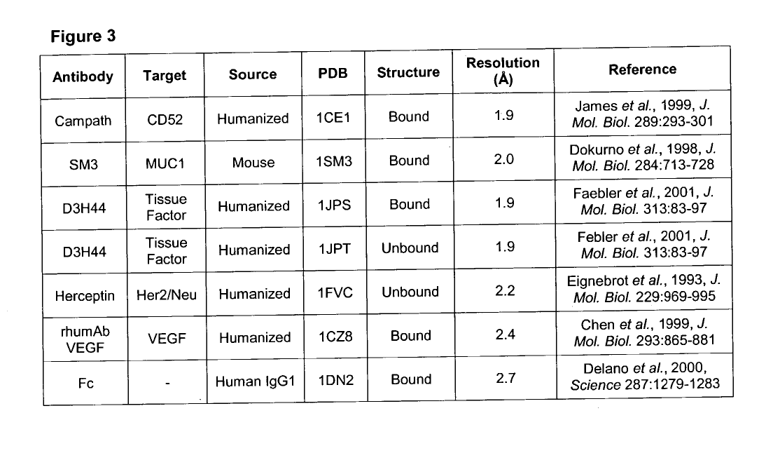

FIG. 3 shows a list of the antibody structures which are used as templates in the provided examples. Unless otherwise noted, the groups of core, surface, and boundary for choice of amino acids considered at variable positions are composed of the following sets of amino acids: core=alanine,.valine, isoleucine, leucine, phenylalanine, tyrosine, tryptophan, and methionine; surface=alanine, serine, threonine, aspartic acid, asparagine, glutamine, glutamic acid, arginine, lysine and histidine; boundary=alanine, serine, threonine, aspartic acid, asparagine, glutamine, glutamic acid, arginine, lysine, histidine, valine, isoleucine, leucine, phenylalanine, tyrosine, tryptophan, and methionine; All or All 20=all 20 natural amino acids.

Stability Optimization

As discussed above, two main strategies for utilizing computational screening methodology to stabilize antibodies are to enhance the stability of individual Ig domains, and enhance interface stability between individual Ig domains.

Domain Stability

The stability of an antibody can be increased by designing more favorable interactions within one or more individual Ig domains. For an antibody which is made up of more than one Ig domain, each individual Ig domain can be engineered for greater stability. Thus for example, for a human, mouse, rat, or rabbit antibody, stability can be improved by stabilizing one or more of domains V_{H}, V_{L}, Cγ1, C_{L}, Cγ2, and Cγ3.

### Example 1

Campath V_{H} Domain Stabilization

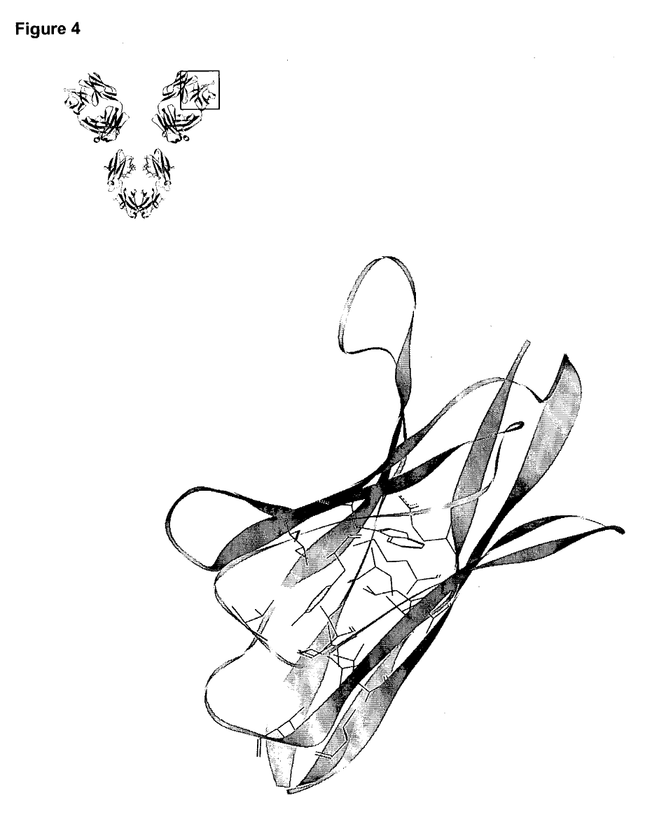

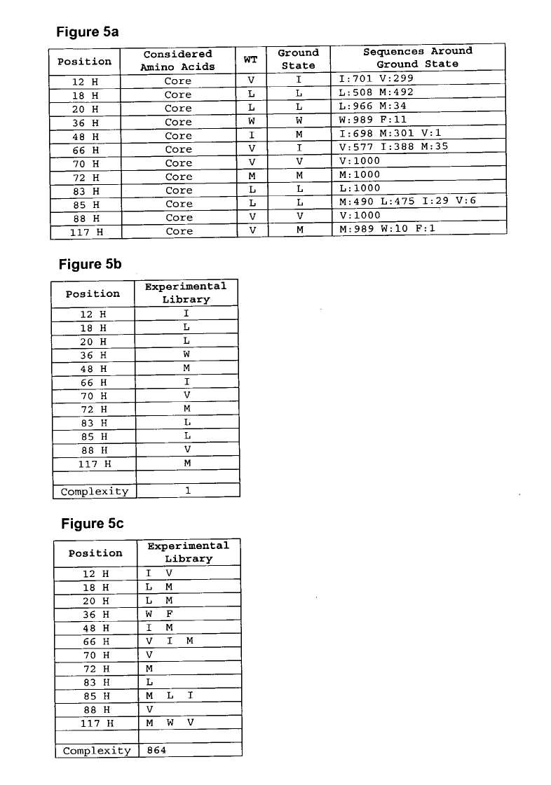

The heavy chain variable domain (V_{H}) of Campath was stabilized using computational screening methods to design more favorable interactions within the interior of the protein. Campath is a humanized antibody that is currently marketed for treatment for B-cell chronic lymphocytic leukemia. The high resolution structure is available of the complex of the Campath Fab with its target antigen, a peptide from the cell surface protein CD52. This structure, PDB accession code 1CE1, served as the template for design calculations. The V_{H} domain of Campath, and most antibodies, has an extensive interior which is critical to its stability. This interior can be thought of as being made up of two separate hydrophobic cores which are separated by the central disfulfide bond. These cores are referred to as the upper core and lower core, with the directional distinction being defined when the CDRs are facing upward as shown in FIG. 4. As will be appreciated by those skilled in the art, packing interactions between the hydrophobic residues which make up these cores play a key role in V_{H} stability, and thus in the stability of any antibody to which V_{H} belongs. Computational screening was applied to design more stable packing interactions in the V_{H} lower core. Variable positions were chosen by visual inspection of the 1CE1 structure, and these positions are shown in FIG. 4 and listed in FIG. 5a. Because these positions are almost completely sequestered from solvent, the amino acids considered were chosen as the set belonging to the core classification. The conformations of amino acids at variable positions were represented as a set of backbone-independent side chain rotamers derived from the rotamer library of Dunbrack & Cohen (Dunbrack & Cohen, 1997, Protein Science 6:1661-1681).

The energies of all possible combinations of the considered amino acids at the chosen variable positions were calculated using a force field containing terms describing van der Waals, solvation, electrostatic, and hydrogen bond interactions, and the optimal (ground state) sequence was determined using a DEE algorithm. This ground state, and the WT Campath sequence, are shown in FIG. 5a. The fact that the ground state is very similar to the WT sequence validates the computational screening method. As will be appreciated by those in the art, the predicted lowest energy sequence is not necessarily the true lowest energy sequence because of errors, primarily in the scoring function, coupled with the fact that subtle conformational differences in proteins can result in dramatic differences in stability. However, the predicted ground state sequence is likely to be close to the true ground state, and thus this problem can be hedged by screening variants close in sequence space and in energy around the predicted ground state. Towards this goal, in order to generate a diversity of sequences for an experimental library, a Monte Carlo algorithm was used to evaluate the energies of 1000 similar sequences around the predicted ground state. FIG. 5a shows the output sequence lists from this Monte Carlo search.

These results can be used to generate one or more experimental libraries which can be screened for increased antibody stability. As discussed above, there are a variety of ways to generate an experimental library. Library 1, shown in FIG. 5b is a defined library of just the ground state sequence. Library 2, shown in FIG. 5c, is a combinatorial library in which a 1% cutoff of occupancy has been applied to the Monte Carlo output, that is to say that only amino acid substitutions which occur in 10 or greater variants out of the 1000 Monte Carlo output sequences are included in the library. Because valine does not occur at heavy chain position 117 in the Monte Carlo output, the WT sequence is not represented. It may be judicious to include this valine at 117 H so that the WT amino acids are represent combinatorially in library 2. The combination of all of these substitutions with all other substitutions results in a combinatorial complexity of 864, i.e. there are 864 possible variants in the library.

### Example 2

Campath V_{H} Domain Stabilization

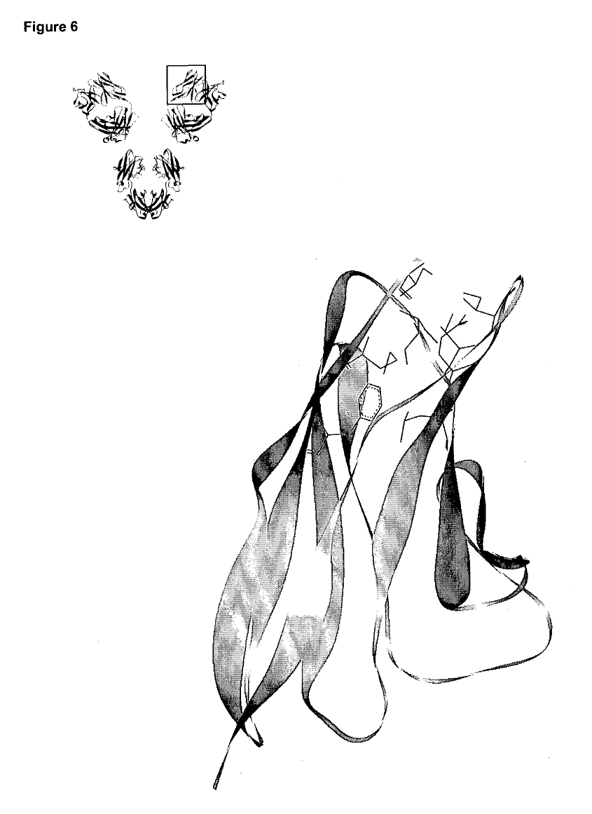

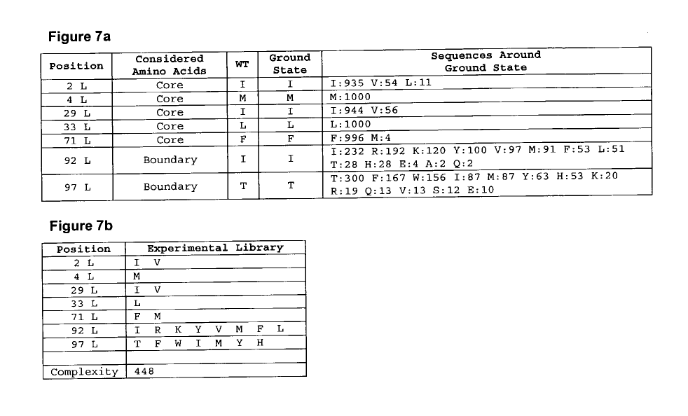

The light chain variable domain (V_{L}) of Campath was also stabilized by using computational screening methods. Like the V_{H} domain, V_{L} has an extensive interior which can be thought of as being made up of an upper and lower core, separated by the central disfulfide bond, shown in FIG. 6. Computational screening was applied to design more stable packing interactions in the V_{L} upper core. Stabilization of the upper core may be less straightforward than the lower core because subtle conformational changes to the upper may more directly impact the conformation of the CDRs, and thus mutations may affect antigen binding. Variable positions were chosen by visual inspection of the 1CE1 structure, and these positions are shown in FIG. 6 and listed in FIG. 7a. For most variable positions, the amino acids conserved were chosen as the set belonging to the core classification because they are sequestered from solvent. Substitutions at two light chain positions, 92 and 97, could potentially make favorable polar interactions, and so amino acids considered for these positions were chosen as the set belonging to the boundary classification. The conformations of amino acids at variable-positions were represented as a set of side chain rotamers derived from a backbone-independent rotamer library.

The CE1 structure was used as the template for design calculations. The energies of all possible combinations of the considered amino acids at the chosen variable positions were calculated using a force field containing terms describing van der Waals, salvation, electrostatic, and hydrogen bond interactions, and the optimal (ground state) sequence was determined using a DEE algorithm. This ground state, and the WT Campath sequence, are shown in FIG. 7a. The fact that the WT sequence is predicted to be the ground state validates the computational screening method. A diversity of sequences for an experimental library was generated by using a Monte Carlo algorithm to evaluate the energies of 1000 similar sequences around the predicted ground state. FIG. 7a shows the output sequence lists from this Monte Carlo search.

These results can be used to generate one or more experimental libraries which can be subsequently screened for increased antibody stability. An experimental library, shown in FIG. 7b, was derived from this set of designed calculations by applying a 5% cutoff of occupancy to the Monte Carlo output, i.e. only amino acid substitutions which occur in 50 or greater variants out of the 1000 Monte Carlo output sequences are included in the library. This combinatorial library has a complexity of 448.

### Example 3

Campath Cγ1 Domain Stabilization

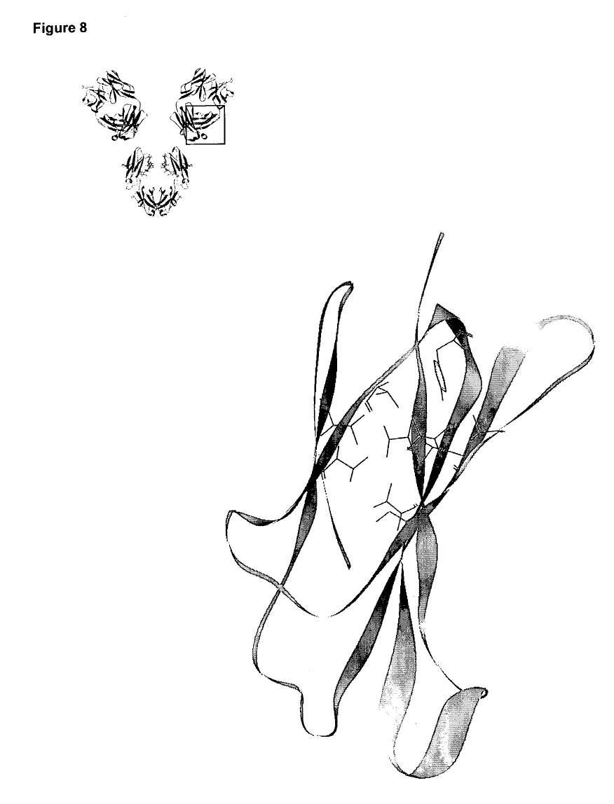

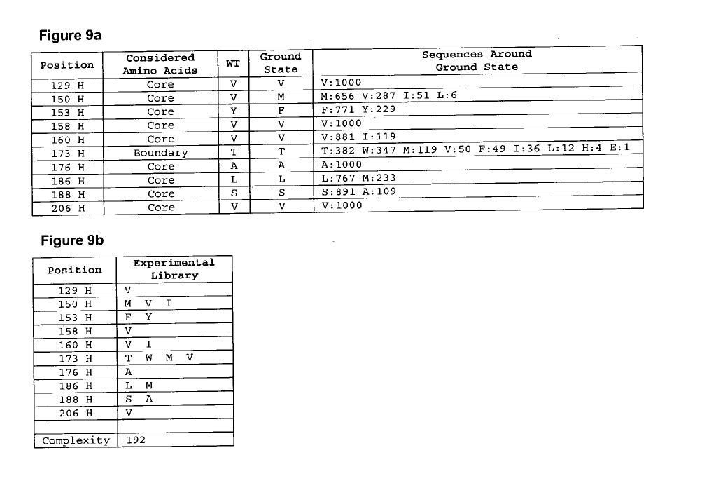

The heavy chain constant domain 1 (Cγ1) is also important to antibody stability. This domain is a part of the antibody constant region, and thus improvements made are widely applicable to antibodies, independent of what antigen is bound at the variable region. The Cγ1 of Campath was stabilized using computational screening methods to design more favorable interactions within the interior of the protein. Like most immunoglobulin domains, Cγ1 has an extensive interior made up of an upper and lower core, separated by the central disfulfide bond, shown in FIG. 8. Computational screening was applied to design more stable packing interaction in the Cγ1 upper core. Variable positions were chosen by visual inspection of the 1CE1 structure, and these positions are shown in FIG. 8 and listed in FIG. 9a. The majority of the chosen core variable positions are sequestered from solvent, and therefore the amino acids conserved were chosen as the set belonging to the core classification. The exception is heavy chain position 173, substitutions at which could potentially make favorable polar interactions, and so amino acids considered for this position were chosen as the set belonging to the boundary classification. The conformations of amino acids at variable positions were represented as a set of side chain rotamers derived from a backbone-independent rotamer library. The CE1 structure was used as the template for design calculations. The energies of all possible combinations of the considered amino acids at the chosen variable positions were calculated using a force field containing terms describing van der Waals, solvation, electrostatic, and hydrogen bond interactions, and the optimal (ground state) sequence was determined using a DEE algorithm. This ground state, and the WT Campath sequence, are shown in FIG. 9a. The fact that the predicted ground state sequence is very similar to the WT sequence validates the computational screening method. A diversity of sequences for an experimental library was generated by using a Monte Carlo algorithm to evaluate the energies of 1000 similar sequences around the predicted ground state. FIG. 9a shows the output sequence lists from this Monte Carlo search.

These results can be used to generate one or more experimental libraries which can be subsequently screened for increased antibody stability. An experimental library, shown in FIG. 9b, was derived from this set of designed calculations by applying a 5% cutoff of occupancy to the Monte Carlo output, i.e. only amino acid substitutions which occur in 50 or greater variants out of the 1000 Monte Carlo output sequences are included in the library. This combinatorial library has a complexity of 192.

### Example 4

Fc Cγ2 Domain Stabilization

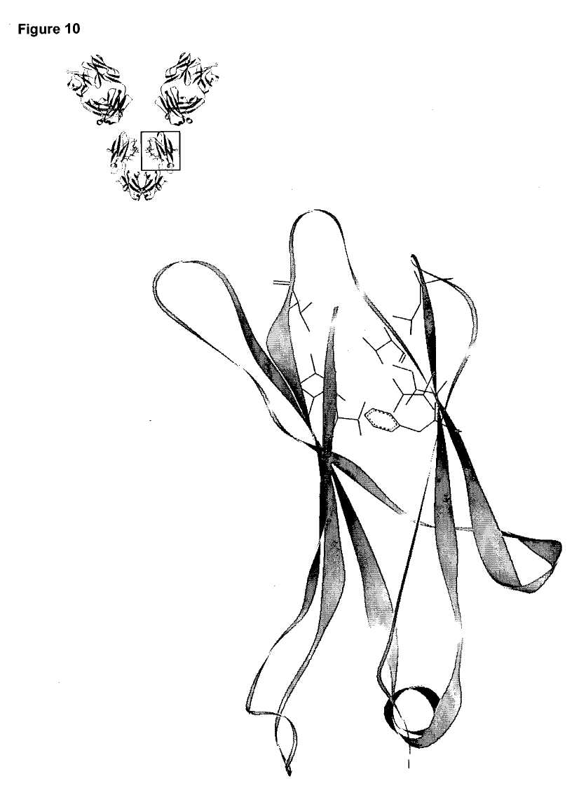

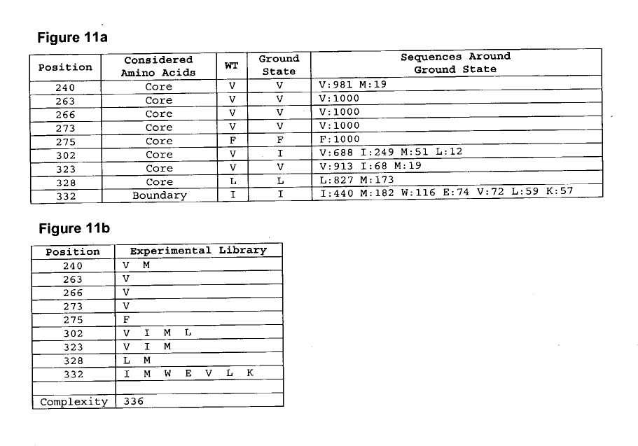

The heavy chain constant domain 2 (Cγ2) is also important to antibody stability. This domain is part of the antibody Fc region, and thus improvements made are widely applicable to antibodies, independent of what antigen is bound at the variable region. The Fc Cγ2 domain was stabilized using computational screening methods to design more favorable interactions within the interior of the protein. The high resolution structure of human Fc has been solved. This structure, PDB accession code 1DN2, served as the template for design calculations. Like most immunoglobulin domains, Cγ2has an extensive interior made up of an upper and lower core, separated by the central disfulfide bond, shown in FIG. 10. Computational screening was applied to design more stable packing interactions in the Cγ2 upper core. Variable positions were chosen by visual inspection of the 1DN2 structure, and these positions are shown in FIG. 10 and listed in FIG. 11a. The majority of the chosen core variable positions are sequestered from solvent, and therefore the amino acids conserved were chosen as the set belonging to the core classification. The exception is position 332, substitutions at which could potentially make favorable polar interactions, and so amino acids considered for this position were chosen as the set belonging to the boundary classification. The conformations of amino acids at variable positions were represented as a set of side chain rotamers derived from a backbone-independent rotamer library.

The energies of all possible combinations of the considered amino acids at the chosen variable positions were calculated using a force field containing terms describing van der Waals, solvation, electrostatic, and hydrogen bond interactions, and the optimal (ground state) sequence was determined using a DEE algorithm. This ground state, and the WT Fc sequence, are shown in FIG. 11a. The fact that the predicted ground state sequence is very similar to the WT sequence validates the computational screening method. A diversity of sequences for an experimental library was generated by using a Monte Carlo algorithm to evaluate the energies of 1000 similar sequences around the predicted ground state. FIG. 11a shows the output sequence lists from this Monte Carlo search.

These results can be used to generate one or more experimental libraries which can be screened for increased antibody stability. An experimental library, shown in FIG. 11b, was derived directly from this set of designed calculations, i.e. no cutoff criteria were applied. This combinatorial library has a complexity of 336.

### Example 5

Fc Cγ3 Domain Stabilization

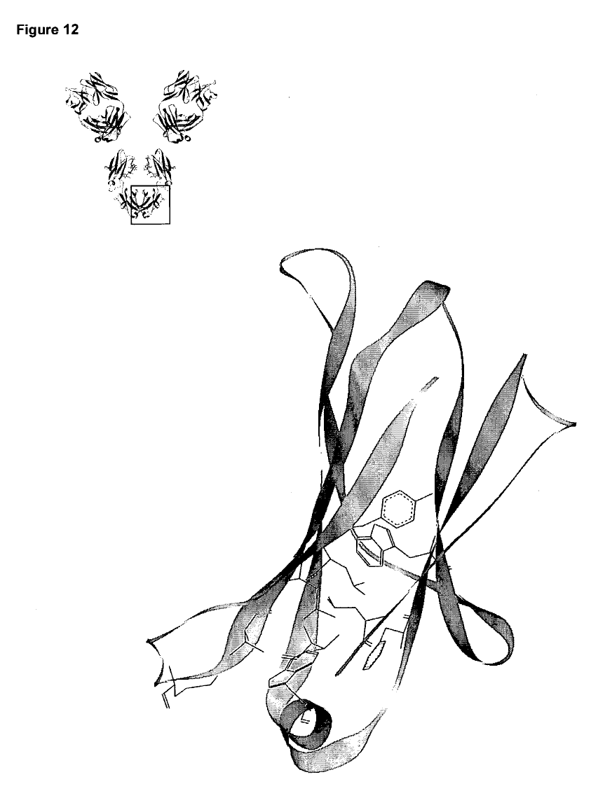

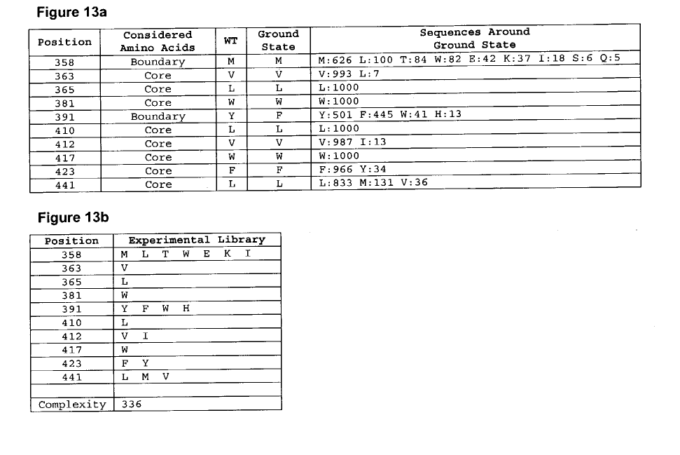

The heavy chain constant domain 3 (Cγ3) is also important to antibody stability. This domain is part of the antibody Fc region, and thus improvements made are widely applicable to antibodies, independent of what antigen is bound at the variable region. The Fc Cγ3 domain was stabilized by using computational screening methods to design more favorable interactions within the interior of the protein. Like most immunoglobulin domains, Cγ2 has an extensive interior made up of an upper and lower core, separated by the central disfulfide bond, shown in FIG. 12. Computational screening was applied to design more stable packing interaction in the Cγ3 lower core. Variable positions were chosen by visual inspection of the 1DN2 structure, and these positions are shown in FIG. 12 and listed in FIG. 13a. The majority of the chosen core variable positions are sequestered from solvent, and therefore the amino acids conserved were chosen as the set belonging to the core classification. The exceptions are positions 358 and 391, substitutions at which could potentially make favorable polar interactions, and so amino acids considered for these positions were chosen as the set belonging to the boundary classification. The conformations of amino acids at variable positions were represented as a set of side chain rotamers derived from a backbone-independent rotamer library. 1DN2 was used as the structural template for design calculations. The energies of all possible combinations of the considered amino acids at the chosen variable positions were calculated using a force field containing terms describing van der Waals, salvation, electrostatic, and hydrogen bond interactions, and the optimal (ground state) sequence was determined using a DEE algorithm. This ground state, and the WT Fc sequence, are shown in FIG. 13a. The fact that the predicted ground state sequence is very similar to the WT sequence validates the computational screening technology. A diversity of sequences for an experimental library was generated by using a Monte Carlo algorithm to evaluate the energies of 1000 similar sequences around the predicted ground state. FIG. 13a shows the output sequence lists from this Monte Carlo search.

These results can be used to generate one or more experimental libraries which can be screened for increased antibody stability. An experimental library, shown in FIG. 13b, was derived from this set of designed calculations by applying a 1% cutoff of occupancy to the Monte Carlo output, i.e. only amino acid substitutions which occur in 10 or greater variants out of the 1000 Monte Carlo output sequences are included in the library. This combinatorial library has a complexity of 336.

Interface Stability

The stability of an antibody can be increased by designing more favorable interactions between individual Ig domains at inter-Ig domain interfaces. For example, as can be seen in FIG. 1, for human antibodies there are five interdomain interfaces that can be optimized using computational screening methodology: V_{H}V_{L}, Cγ1/C_{L}, V_{H}/Cγ1, V_{L}/C_{L}, and Cγ3/Cγ3.

### Example 6

rhumAb VEGF V_{H}/V_{L} Interface Stabilization

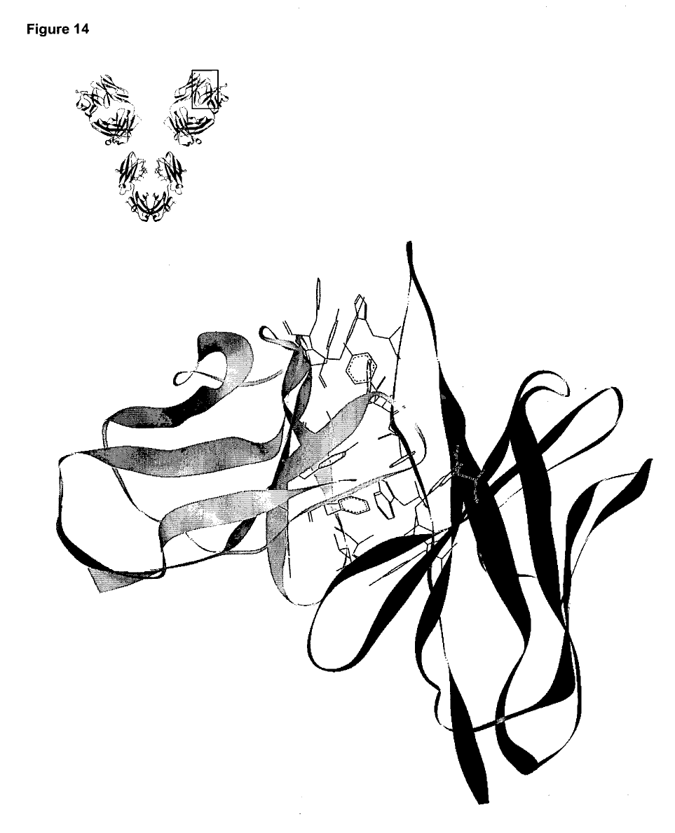

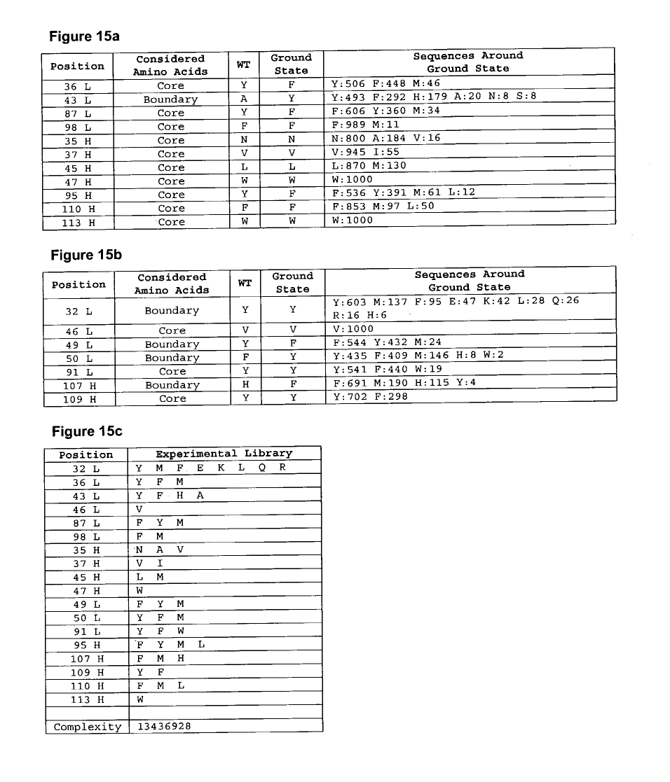

The stability of the interface between the V_{H} and V_{L} domains is critical to antibody stability. The antibody rhumAb VEGF was stabilized by enhancing the interaction between the V_{H} and V_{L} domains by using computational screening methods to design more favorable interactions between the residues which make up this interface. rhumAb VEGF is a humanized antibody that is currently in clinical development for treatment of a variety of cancers. The high resolution structure is available of the complex of the rhumAb VEGF Fab fragment with its target antigen, the vascular endothelial growth factor (VEGF). This structure, PDB accession code 1CZ8, served as the template for design calculations. The V_{H}/V_{L} interface of rhumAb VEGF is shown in FIG. 14. Variable positions were chosen by visual inspection of the 1CZ8 structure, and these positions are shown in FIG. 14 and listed in FIGS. 15a and 15b. For rhumAb VEGF, the interface can be separated into two somewhat independent sets of residues, and thus it was possible to carry out computational screening in two separate sets of design calculations. The sets of amino acids considered at variable positions were chosen subjectively by visual inspection of the 1CZ8 structure. The conformations of amino acids at variable positions were represented as a set of side chain rotamers derived from a backbone-independent rotamer library.

The 1CZ8 structure was used as the template for design calculations. For both sets of calculations, the energies of all possible combinations of the considered amino acids at the chosen variable positions were calculated using a force field containing terms describing van der Waals, salvation, electrostatic, and hydrogen bond interactions, and the optimal (ground state) sequences were determined using a DEE algorithm. These ground states, and the WT rhumAb VEGF sequence, are shown in FIGS. 15a and 15b. The fact that the predicted ground state sequences are very similar to the WT sequence validates the computational screening method. A diversity of sequences for an experimental library was generated by using a Monte Carlo algorithm to evaluate the energies of 1000 similar sequences around the predicted ground states. FIGS. 15a and 15b show the output sequence lists from these Monte Carlo searches.

These results can be used to generate one or more experimental libraries which can be screened for increased antibody stability. An experimental library, shown in FIGS. 15c, was derived by applying a 1% cutoff of occupancy to the Monte Carlo output from each set of calculations, and then these primary libraries were subsequently combined to generate a secondary library with mutations at all positions. This combinatorial library has a complexity of 1.3×10⁷.

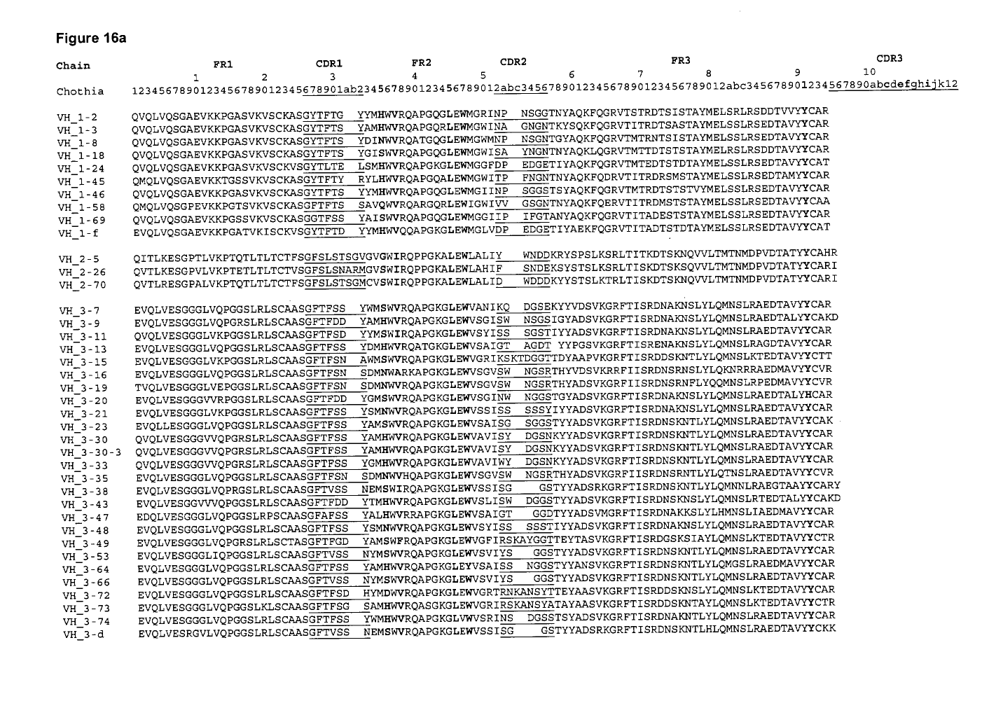

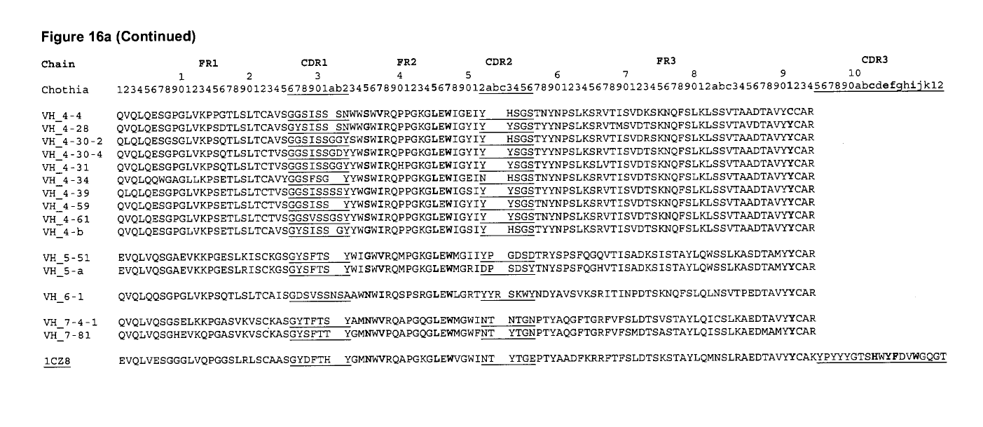

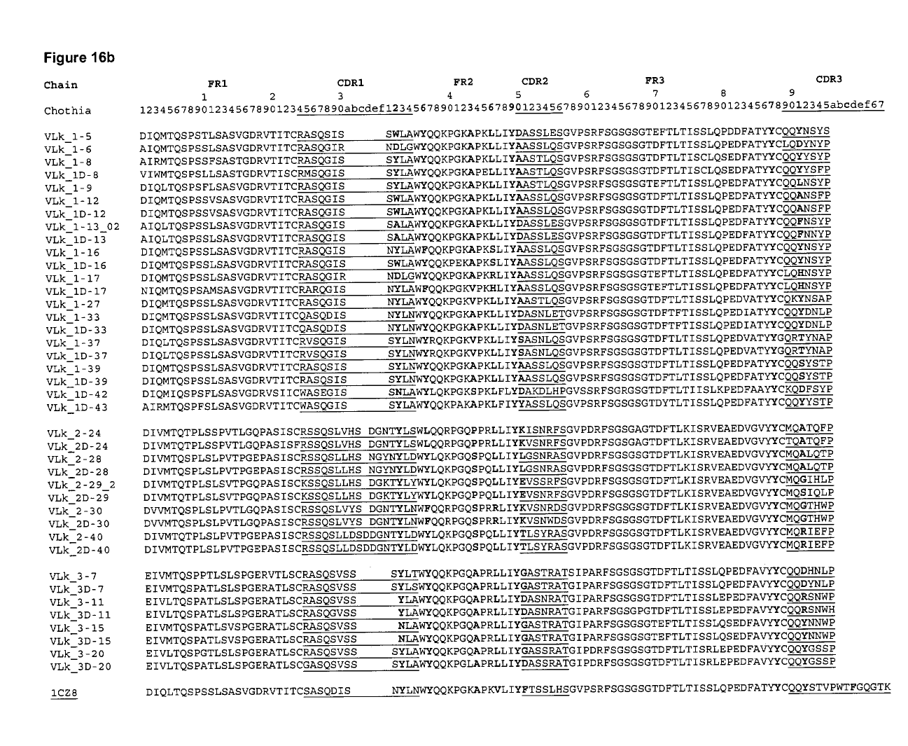

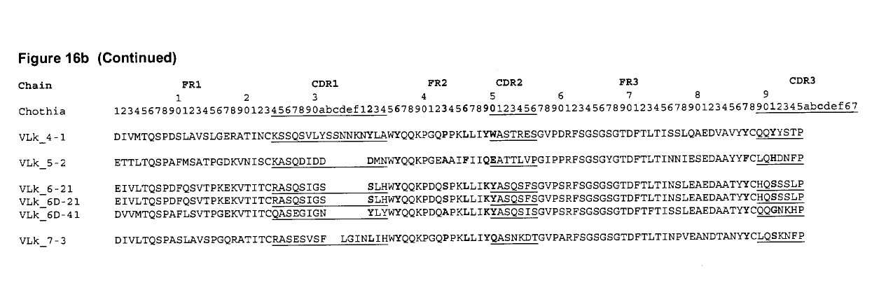

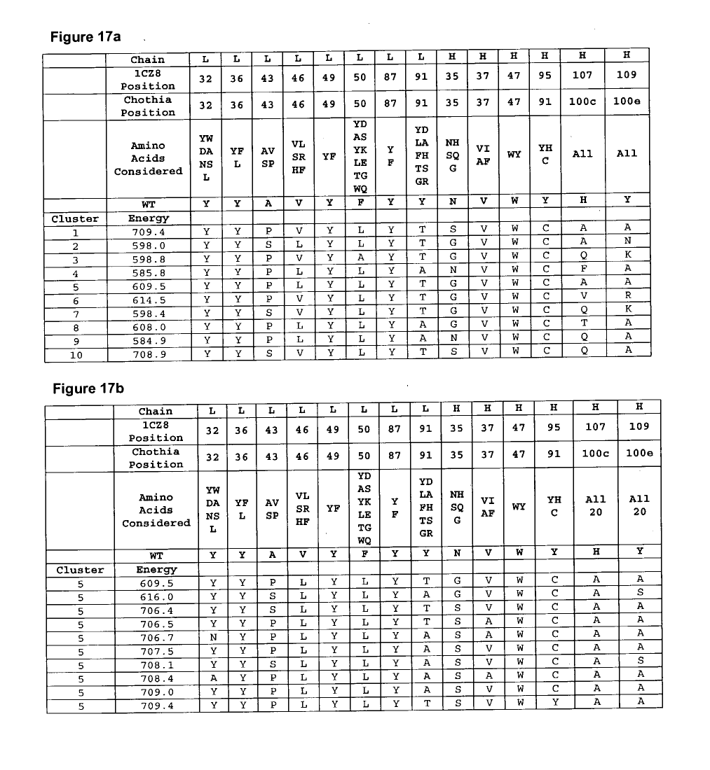

Because of the number of residues involved in mediating this interface, it may be beneficial to reduce the complexity of the design calculations. As discussed above, sequence information can be used to guide the choice of variable positions and the set of amino acids considered at those positions. The use of sequence information here will enable the complexity of the computational problem to be reduced while ensuring that the remaining diversity sampled is of high quality, in terms of the structural, functional, and immunogenic fidelity of the antibody. FIGS. 16a and 16b show the 1CZ8 heavy and light chain variable chain sequences aligned with the human V_{H} and V_{L} kappa germ line sequences. A new design calculation using this information was run to stabilize the V_{H}/V_{L} interface. The sequence information was first used to reevaluate the list of variable positions. A subset of the positions in FIGS. 15a and 15b were chosen based on the degree of variability at each position in the germ line. Those positions with one type of amino acid in the majority of the sequences, or for which there is no sequence information, were not allowed to vary in the calculation. This new set is shown in FIG. 17a. Light chain position 98 and heavy chain positions 45, 110, and 113 were not variable positions in this calculation, but were floated. The sequence information was also used to choose the set of amino acids to be considered at variable positions in the new design calculation. All amino acids, and only those amino acids, which appear at each variable position in the germ line were considered in the new design calculation. For variable positions in the light and heavy chain CDR3s, for which no sequence information is available, all 20 amino acids were considered. This set of considered amino acids is shown in FIG. 17a.

The 1CZ8 structure was used as the template for design calculations. In this new calculation, energies of all possible combinations were not precalculated. Instead, a genetic algorithm was used to screen for low energy sequences, with energies being calculated during each round of “evolution” only for those sequences being sampled. The conformations of amino acids at variable and floated positions were represented as a set of side chain rotamers derived from a backbone-independent rotamer library using a flexible rotamer model (Mendes et. al., 1999, Proteins: Structure, Function, and Genetics 37:530-543). Energies were calculated using a force field containing terms describing van der Waals, solvation, electrostatic, and hydrogen bond interactions. This calculation generated a list of 300 sequences which are predicted to be low in energy. Clustering was performed to facilitate analysis of the results and library generation. The 300 output sequences were clustered computationally into 10 groups of similar sequences using a nearest neighbor single linkage hierarchical clustering algorithm to assign sequences to related groups based on similarity scores (Diamond, R., Coordinate-Based Cluster Analysis, Acta Cryst. 1995, D51, 127-135.). That is, all sequences within a group are most similar to all other sequences within the same group and less similar to sequences in other groups. The lowest energy sequence from each of these ten clusters, used here as a representative of each group, is presented in FIG. 17a.

These results can be used to generate one or more experimental libraries which can be subsequently screened for increased antibody stability. An experimental library can be derived directly from the representative cluster group sequences. Thus FIG. 17a provides a 10 sequence experimental library. To efficiently use experimental resources, this library size of 10 variants could be screened first, followed by subsequent screening of sequences or a subset of sequences within the group to which the experimentally determined most favorable variant belongs. For example, if variant 5 (i.e. the lowest energy sequences from cluster group 5) was found to be most favorable, all of the sequences of cluster group 5 could be subsequently screened. The 14 sequences in group 5 are presented in FIG. 17b as an example of such an experimental library.

### Example 7

Herceptin V_{H}/V_{L} Interface Stabilization

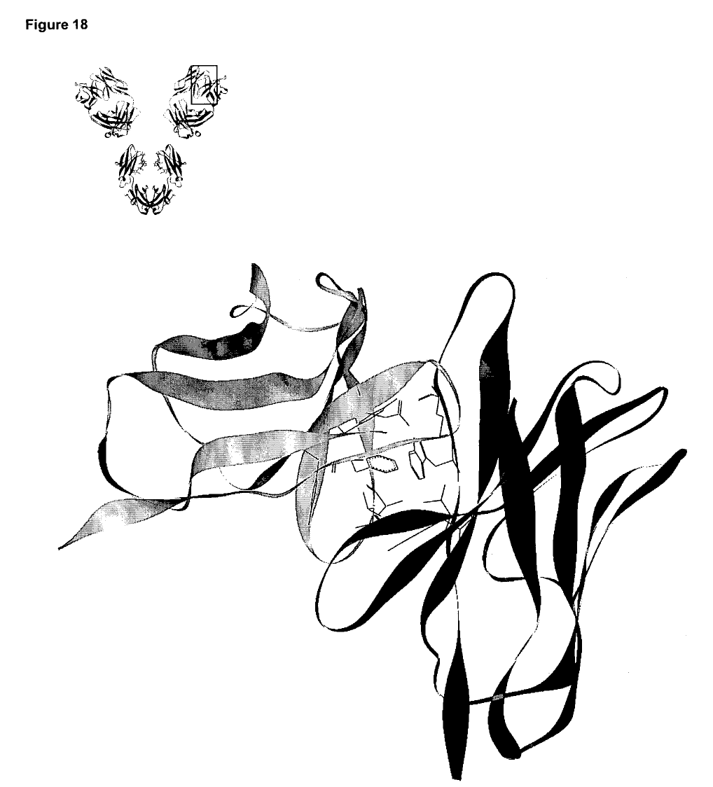

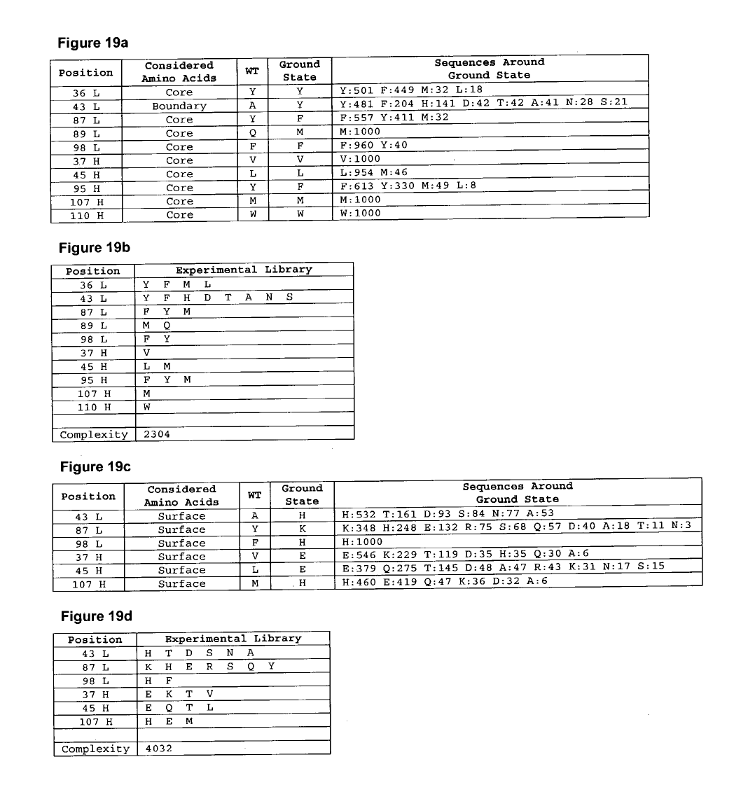

The interface between the V_{H} and V_{L} domains of the antibody Herceptin was also stabilized. More favorable interactions between the V_{H} and V_{L} domains were designed using computational screening methods. Herceptin, which targets the extracellular domain of the proto-oncogene Her2/neu gene product, also known as erbB2, is a humanized antibody that is currently marketed for treatment for breast cancer. The high resolution structure is available of uncomplexed Herceptin scFv. This structure, PDB accession code 1FVC, served as the template for design calculations. The V_{H}/V_{L} interface of Herceptin is shown in FIG. 18. Variable positions were chosen by visual inspection of the 1FVC structure, and these positions are shown in FIG. 18 and listed in FIG. 19a. The majority of the chosen core variable positions are sequestered from solvent, and therefore the amino acids conserved were chosen as the set belonging to the core classification. The exception is light chain position 43, substitutions at which could potentially make favorable polar interactions, and so amino acids considered for this position were chosen as the set belonging to the boundary classification. The conformations of amino acids at variable positions were represented as a set of side chain rotamers derived from a backbone-independent rotamer library.

The 1FVC structure was used as the structural template for design calculations. The energies of all possible combinations of the considered amino acids at the chosen variable positions were calculated using a force field containing terms describing van der Waals, solvation, electrostatic, and hydrogen bond interactions, and the optimal (ground state) sequence was determined using a DEE algorithm. This ground state, and the WT Herceptin sequence, are shown in FIG. 19a. The fact that the predicted ground state sequence is very similar to the WT sequence validates the computational screening technology. A diversity of sequences for an experimental library was generated by using a Monte Carlo algorithm to evaluate the energies of 1000 similar sequences around the predicted ground state. FIG. 19a shows the output sequence list from this Monte Carlo search. These results can be used to generate one or more experimental libraries which can be subsequently screened for increased antibody stability. An experimental library, shown in FIG. 19b, was derived by applying a 1% cutoff of occupancy to the Monte Carlo output from each set of calculations, i.e. only amino acid substitutions which occur in 10 or greater variants out of the 1000 Monte Carlo output sequences are included in the library. Additionally, the glutamine was added at light chain position 89 so that the WT sequence is represented. This combinatorial library has a complexity of 5184. In the above calculation, for all but one variable position only nonpolar amino acids were considered. As discussed above, nonpolar residues have a higher tendency to aggregate than polar residues, and therefore nonpolar amino acids at the interdomain interface can result in a greater nonreversibility of the unbinding/binding transition. Design of a stable interface with greater polar character may thus provide greater thermodynamic reversibility and improved solubility. Another Herceptin V_{H}/V_{L} interface calculation was carried out in which the amino acids considered were chosen as the set belonging to the surface classification. A number of nonpolar interactions, however, appear critical to this interface, both by visual inspection and by their level of conservation in the aligned germ lines (FIGS. 2a and 2b). These positions, including light chain positions 36 and 89, and heavy chain positions 95 and 110, were floated in the new calculation. The remaining set of variable positions is shown in FIG. 19c.

The 1FVC structure was used as the template for design calculations. The energies of all possible combinations of the considered amino acids at the chosen variable positions were calculated using a force field containing terms describing van der Waals, solvation, electrostatic, and hydrogen bond interactions, and the optimal (ground state) sequence was determined using a DEE algorithm. This ground state, and the WT Herceptin sequence, are shown in FIG. 19c. The fact that the predicted ground state sequence is very similar to the WT sequence validates the computational screening technology. A diversity of sequences for an experimental library was generated by using a Monte Carlo algorithm to evaluate the energies of 1000 similar sequences around the predicted ground state. FIG. 19c shows the output sequence list from this Monte Carlo search.

These results can be used to generate one or more experimental libraries which can be screened for increased antibody stability. An experimental library, shown in FIG. 19d, was derived by applying a 5% cutoff of occupancy to the Monte Carlo output from each set of calculations, i.e. only amino acid substitutions which occur in 50 or greater variants out of the 1000 Monte Carlo output sequences are included in the library. Additionally, the WT residues were added to the library so that the sequence space sampled experimentally also includes interfaces made up of favorable polar and nonpolar residues at these positions. This combinatorial library has a complexity of 4032.

### Example 8

rhumAb VEGF C_{L}/Cγ1 Interface Stabilization

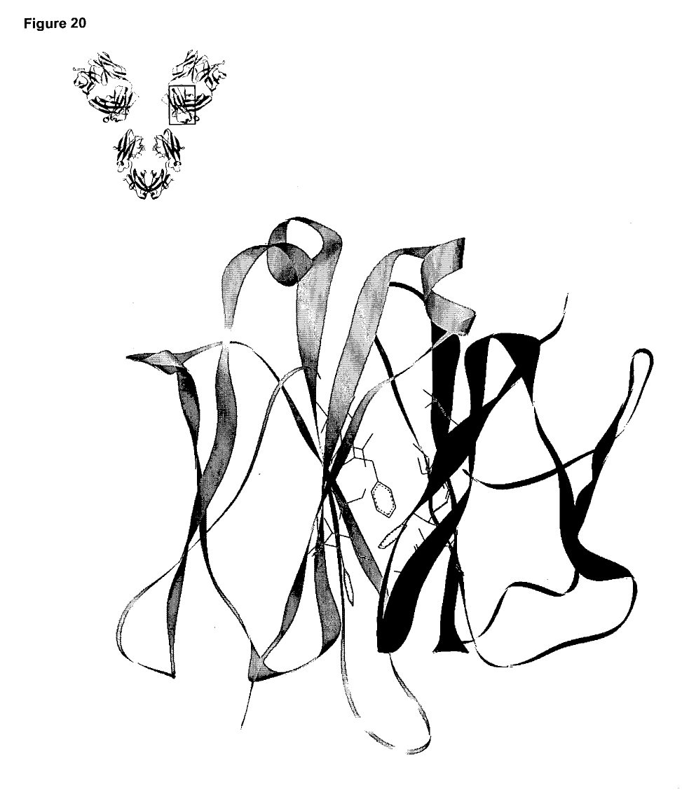

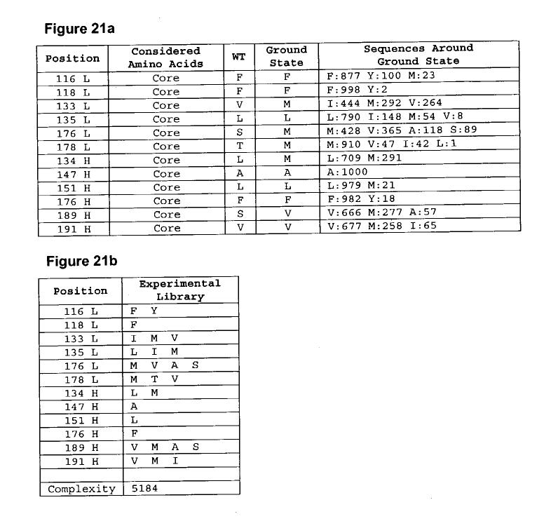

The interface between the C_{L} and Cγ1 domains can also be stabilized using computational screening. More favorable interactions were designed between residues which make up the rhumAb VEGF C_{L}/Cγ1 interface. The C_{L}/Cγ1 interface of rhumAb VEGF is shown in FIG. E8. Variable positions were chosen by visual inspection of the 1CZ8 structure, and these positions are shown in FIG. 20 and listed in FIG. 21a. Because these positions are almost completely sequestered from solvent, the amino acids considered were chosen as the set belonging to the core classification, even for 176, 178, and 189 which are polar amino acids in the WT sequence. The WT amino acids were, however, also considered at these positions. The conformations of amino acids at variable positions were represented as a set of side chain rotamers derived from a backbone-independent rotamer library. The 1CZ8 structure was used as the template for design calculations. The energies of all possible combinations of the considered amino acids at the chosen variable positions were calculated using a force field containing terms describing van der Waals, solvation, electrostatic, and hydrogen bond interactions, and the optimal (ground state) sequence was determined using a DEE algorithm. This ground state, and the WT rhumAb VEGF sequence, are shown in FIG. 21a. The fact that the predicted ground state sequence is very similar to the WT sequence validates the computational screening method. A diversity of sequences for an experimental library was generated by using a Monte Carlo algorithm to evaluate the energies of 1000 similar sequences around the predicted ground state. FIG. 21a shows the output sequence list from this Monte Carlo search. These results can be used to generate one or more experimental libraries which can be subsequently screened for increased antibody stability. An experimental library, shown in FIG. 21b, was derived by applying a 5% cutoff of occupancy to the Monte Carlo output from each set of calculations, i.e. only amino acid substitutions which occur in 50 or greater variants out of the 1000 Monte Carlo output sequences are included in the library. Three additional amino acids were added to this library: threonine and serine were added to light chain position 178 and heavy chain position 189 respectively so that all polar residues are represented in the library, and the valine at light chain position 178 was also included even though it did not make the 5% cutoff. As is known in the art, valine is a good nonpolar substitution for threonine because the two have nearly identical size and shape. This combinatorial library has a complexity of 5184.

### Example 9

Fc Cγ3/Cγ3 Interface Stabilization

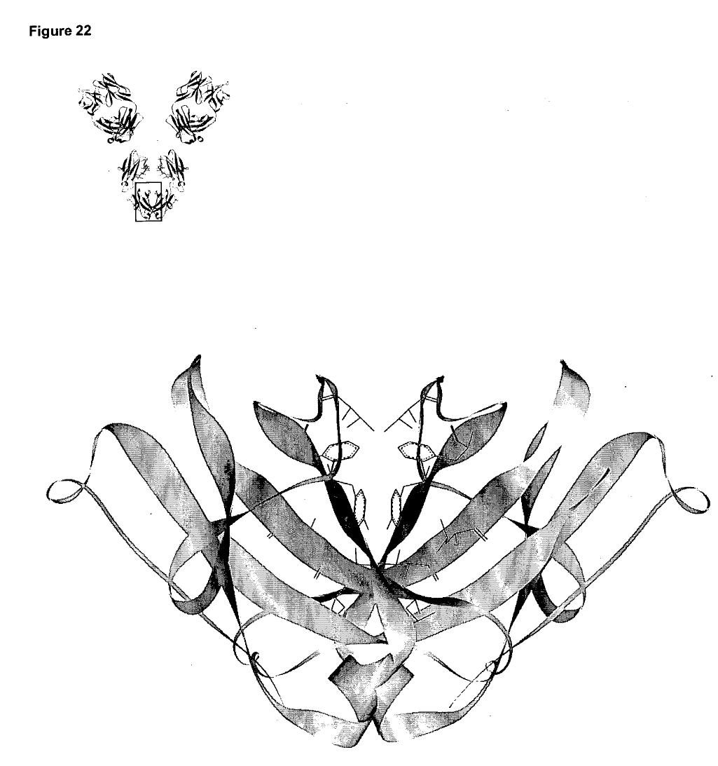

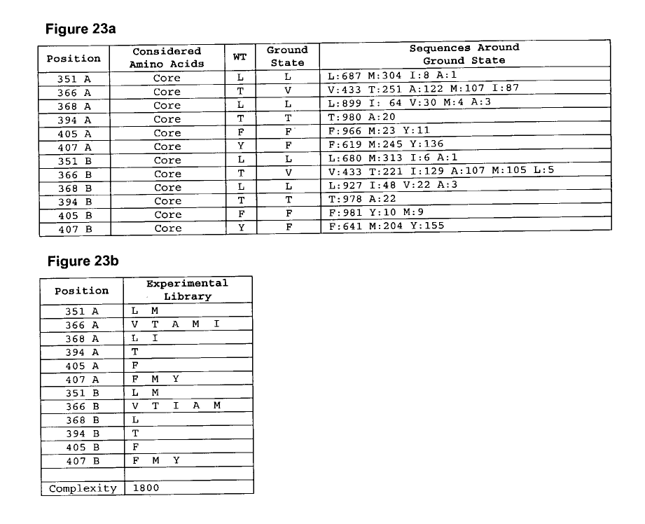

The interface between the Cγ3 domains can also be stabilized using computational screening. Again, because this domain is a part of the antibody Fc region, improvements made are widely applicable to antibodies, independent of what antigen is bound at the variable region. More favorable interactions were designed between residues which make up the Fc Cγ3/Cγ3 interface. Variable positions were chosen by visual inspection of the 1DN2 structure, and these positions are shown in FIG. 22 and listed in FIG. 23a. Because these positions are almost completely sequestered from solvent, the amino acids considered were chosen as the set belonging to the core classification, although the WT amino acid was included at each position. The conformations of amino acids at variable positions were represented as a set of side chain rotamers derived from a backbone-independent rotamer library.

The 1DN2 structure was used as the template for design calculations. The energies of all possible combinations of the considered amino acids at the chosen variable positions were calculated using a force field containing terms describing van der Waals, solvation, electrostatic, and hydrogen bond interactions, and the optimal (ground state) sequence was determined using a DEE algorithm. This ground state, and the WT Fc sequence, are shown in FIG. 23a. The fact that the predicted ground state sequence is very similar to the WT sequence validates the computational screening method. A diversity of sequences for an experimental library was generated by using a Monte Carlo algorithm to evaluate the energies of 1000 similar sequences around the predicted ground state. FIG. 23a shows the output sequence list from this Monte Carlo search.

These results can be used to generate one or more experimental libraries which can be subsequently screened for increased antibody stability. An experimental library, shown in FIG. 23b, was derived by applying a 5% cutoff of occupancy to the Monte Carlo output from each set of calculations, i.e. only amino acid substitutions which occur in 50 or greater variants out of the 1000 Monte Carlo output sequences are included in the library. This combinatorial library has a complexity of 1800.

Solubility Optimization

As discussed above, computational screening methods can be used to optimize the solubility of antibodies by designing favorable, more soluble substitutions at surface exposed nonpolar residues. Residues which can be replaced include residues which are exposed to solvent on individual Ig domains, including V_{H}, V_{L}, Cγ1, C_{L}, Cγ2, and Cγ3 as well as the linkers and/or hinges that connect them, or which lie at the interface between Ig domains.

### Example 10

Campath Solubility Optimization

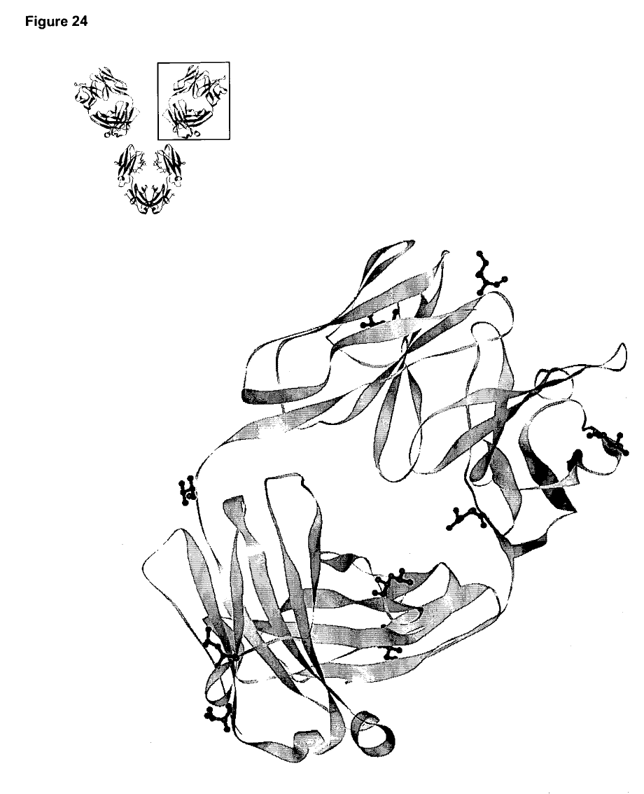

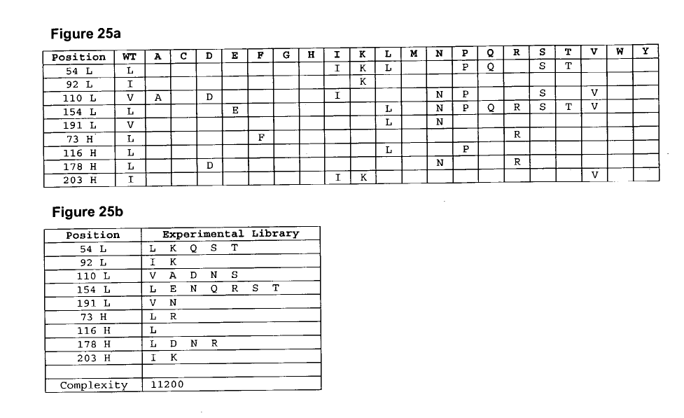

All four Ig domains of the Campath Fab antibody fragment were optimized for greater solubility using computational screening. Computational screening was applied to evaluate the replacement of all exposed nonpolar residues on these domains, including V_{H}, V_{L}, Cγ1, C_{L}, with all 20 amino acids. Variable positions were chosen by visual inspection of the 1CE1 structure, and include exposed nonpolar residues which are not involved in binding antigen. These positions are shown in FIG. 24 and listed in FIG. 25a. Each of the 20 amino acids was considered at each variable position. The 1CE1 structure was used as the template for design calculations. For each variable position, each of the 20 amino acids was substituted and allowed to sample rotamer conformations derived from a backbone-independent rotamer library using a flexible rotamer model. A genetic algorithm was used to optimize the conformation of each amino acid substitution at each variable position, with energies being calculated during each round of evolution. In this way, the lowest energy rotamer of each substitution was determined and this energy was defined as the energy of substitution for that amino acid at that variable position. Thus this design calculation provided an energy of substitution for each of the 20 amino acids at each variable position. FIG. 25a shows these results. At each variable position, the lowest energy substitution and all amino acid substitutions which are within 1 unit of energy of the lowest energy substitution are shown. Thus FIG. 25a presents the most favorable substitutions for each of the variable positions.

These results can be used to generate one or more experimental libraries which can be subsequently screened for improved antibody solubility. An experimental library was derived from this computational screening output by including the WT amino acid and all favorable polar amino acid substitutions at each variable position. As can be seen, no polar substitutions are predicted to be favorable for heavy chain position 116, and so this position is left as the WT leucine in the library. This experimental library, which has a combinatorial complexity of 11200, is shown in FIG. 25b.

### Example 11

rhumAb VEGF Solubility Optimization

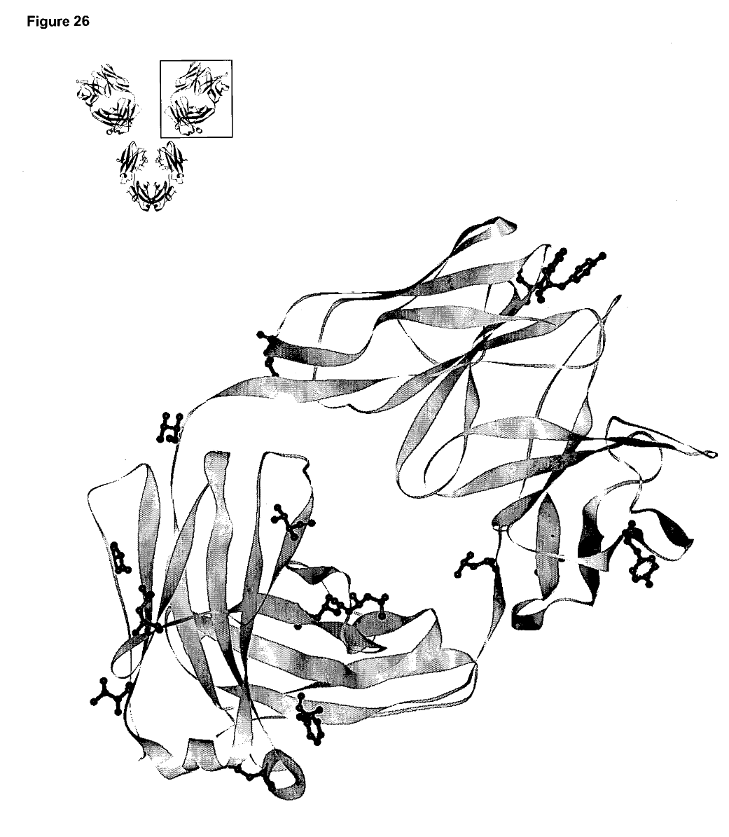

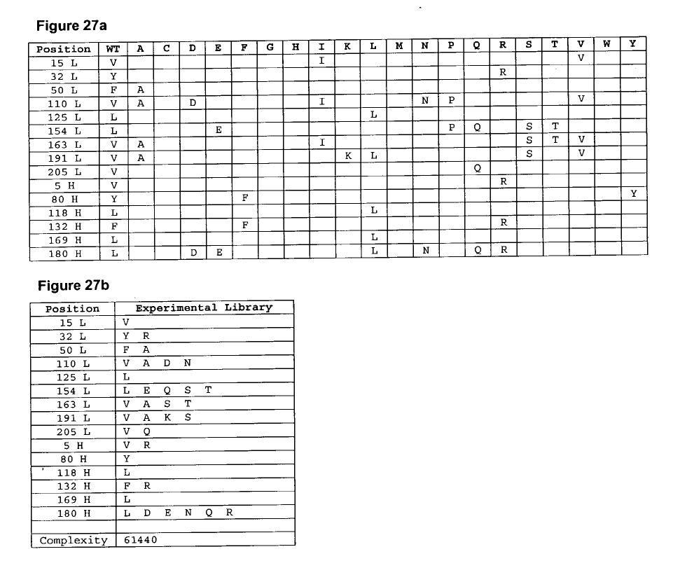

All four Ig domains of the rhumAb VEGF Fab antibody fragment were optimized for greater solubility using computational screening. Computational screening was applied to evaluate the replacement of all exposed nonpolar residues on these domains, including V_{H}, V_{L}, Cγ1, C_{L}, with all 20 amino acids. Variable positions were chosen by visual inspection of the 1CZ8 structure, and include exposed nonpolar residues which are not involved in binding antigen. These positions are shown in FIG. 26 and listed in FIG. 27a. Each of the 20 amino acids was considered at each variable position. The 1CZ8 structure was used as the template for design calculations. For each variable position, each of the 20 amino acids was substituted and allowed to sample rotamer conformations derived from a backbone-independent rotamer library using a flexible rotamer model. A genetic algorithm was used to optimize the conformation of each amino acid substitution at each variable position, with energies being calculated during each round of evolution using a force field containing terms describing van der Waals, solvation, electrostatic, and hydrogen bond interactions. In this way, the lowest energy rotamer of each substitution was determined. This energy was defined as the energy of substitution for that amino acid at that variable position. Thus this design calculation provided an energy of substitution for each of the 20 amino acids at each variable position. FIG. 27a shows these results. At each variable position, the lowest energy substitution and all amino acid substitutions which are within 1 unit of energy of the lowest energy substitution are shown. Thus FIG. 27a presents the most favorable substitutions for each of the variable positions.

These results can be used to generate one or more experimental libraries which can be subsequently screened for improved antibody solubility. An experimental library was derived from this computational screening output by including the WT amino acid and all favorable polar amino acid substitutions at each variable position. As can be seen, no polar substitutions are predicted to be favorable for light chain positions 15 and 125 and heavy chain positions 80, 118, and 169, and so these positions are left as the nonpolar WT amino acids in the library. This experimental library, which has a combinatorial complexity of 61440, is shown in FIG. 27b.

### Example 12

Herceptin Solubility Optimization

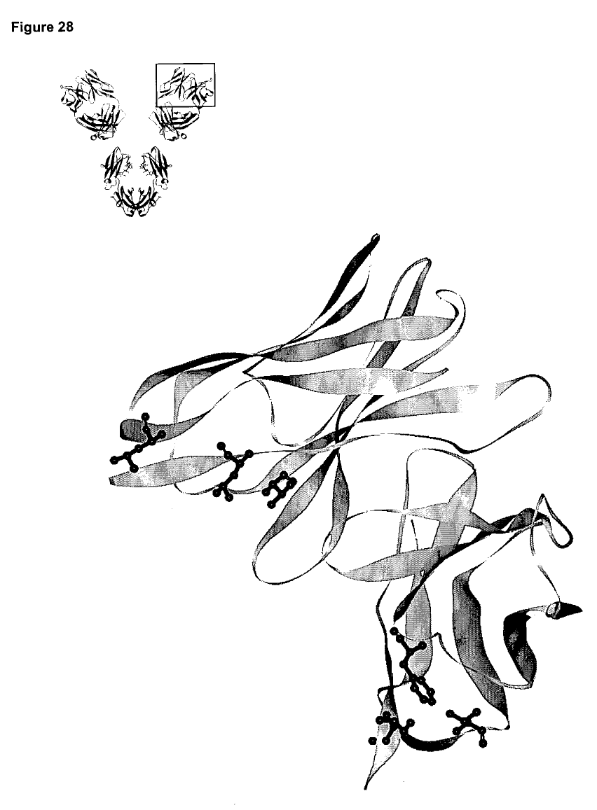

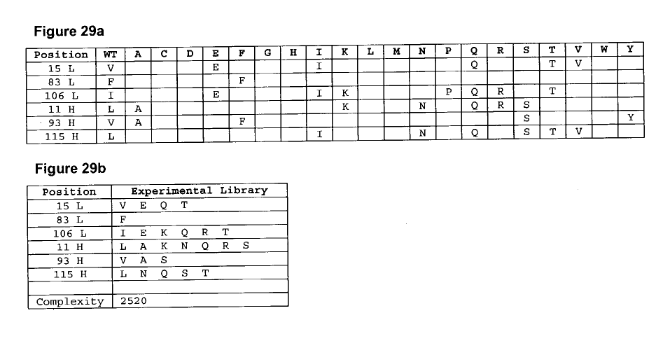

As discussed above, by removing certain regions or domains of an antibody to generate an antibody fragment, nonpolar residues that make up the interface with another Ig domain in the context of a full-length antibody or larger antibody fragment can become exposed. For example, for Herceptin, the V_{H} and V_{L} residues which make up the V_{H}/Cγ1 and V_{L}/C_{L} interfaces are exposed to solvent in an scFv fragment, as is seen in the 1FVC structure. Computational screening was used to engineer favorable, more soluble mutations at these positions for Herceptin. Variable positions were chosen by visual inspection of the 1FVC structure, and include the set of exposed nonpolar residues at the C-terminal end of the V_{H} and V_{L} domains. These positions are shown in FIG. 28 and listed in FIG. 29a. Each of the 20 amino acids was considered at each variable position.

The 1FVC structure was used as the template for design calculations. For each variable position, each of the 20 amino acids was substituted and allowed to sample rotamer conformations derived from a backbone-independent rotamer library using a flexible rotamer model. A genetic algorithm was used to optimize the conformation of each amino acid substitution at each variable position, with energies being calculated during each round of evolution using a force field containing terms describing van der Waals, salvation, electrostatic, and hydrogen bond interactions. In this way, the lowest energy rotamer of each substitution was determined and this energy was defined as the energy of substitution for that amino acid at that variable position. Thus this design calculation provided an energy of substitution for each of the 20 amino acids at each variable position. FIG. 29a shows these results. At each variable position, the lowest energy substitution and all amino acid substitutions which are within 1 unit of energy of the lowest energy substitution are shown. Thus FIG. 29a presents the most favorable substitutions for each of the variable positions.

These results can be used to generate one or more experimental libraries which can be subsequently screened for improved antibody solubility. An experimental library was derived from this computational screening output by including the WT amino acid and all favorable polar amino acid substitutions at each variable position. As can be seen, no polar substitutions are predicted to be favorable for light chain position 83, and so this position is left as the nonpolar WT phenylalanine in the library. This experimental library, which has a combinatorial complexity of 2530, is shown in FIG. 29b.

### Example 13

Fc Solubility Optimization

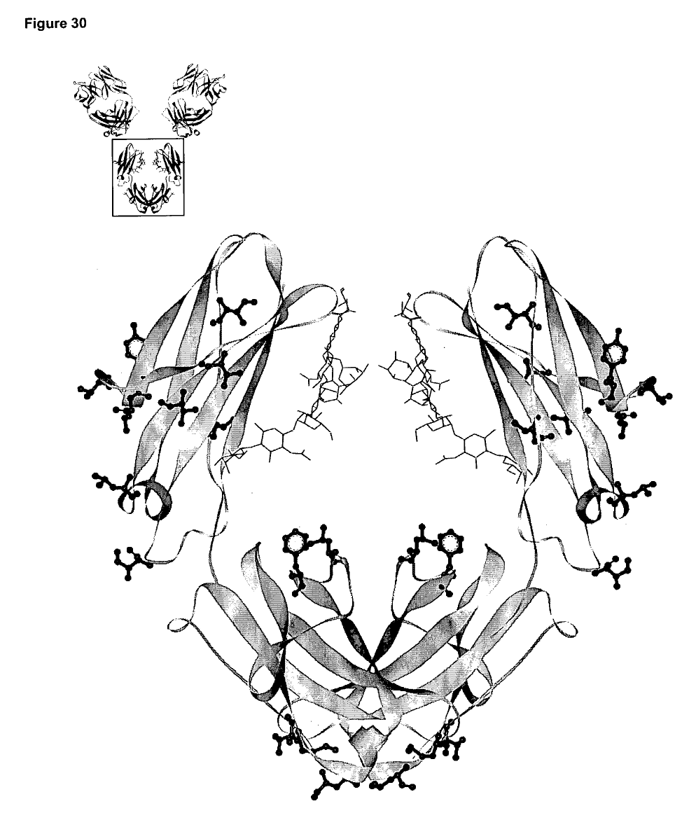

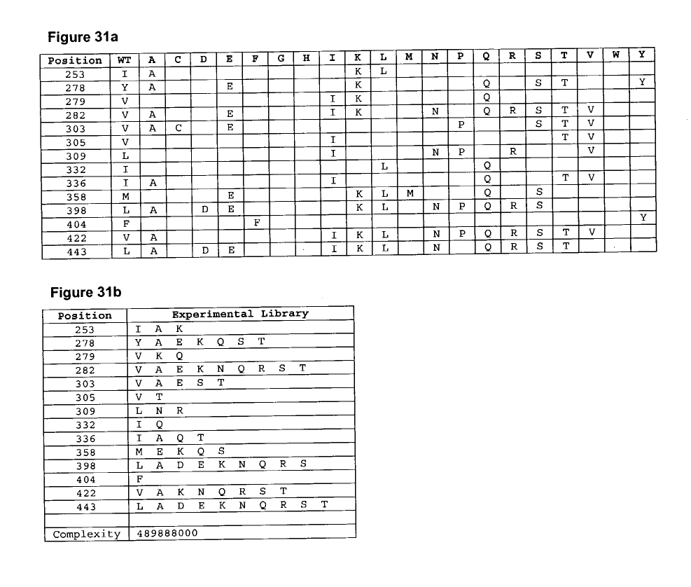

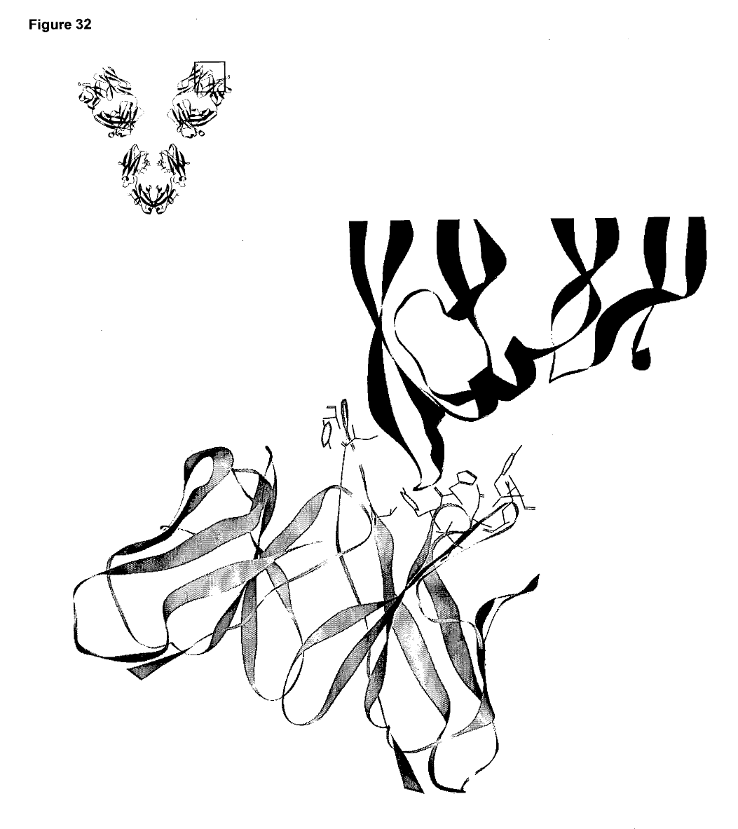

The Fc region was optimized for greater solubility using computational screening. Computational screening was applied to evaluate the replacement of all exposed nonpolar residues on the Cγ2 and Cγ3 domains with all 20 amino acids. Variable positions were chosen by visual inspection of the 1DN2 structure, and include exposed nonpolar residues which are not involved in binding an Fc receptor. For example Met252 and Met428 are involved in binding to FcRn (Martin et al., 2001, Mol. Cell 7:867-877), and Tyr296 and Tyr300 are close to the binding site for FcγRs (Sonderman et al., 2001, J. Mol. Biol. 309:737-749). Therefore these residues, despite being exposed nonpolars, were not included as variable positions. Variable positions are shown in FIG. 30 and listed in FIG. 31a. Each of the 20 amino acids was considered at each variable position.

The 1DN2 structure was used as the template for design calculations. For each variable position, each of the 20 amino acids was substituted and allowed to sample rotamer conformations derived from a backbone-independent rotamer library using a flexible rotamer model. A genetic algorithm was used to optimize the conformation of each amino acid substitution at each variable position, with energies being calculated during each round of evolution using a force field containing terms describing van der Waals, solvation, electrostatic, and hydrogen bond interactions. In this way, the lowest energy rotamer of each substitution was determined. This energy was defined as the energy of substitution for that amino acid at that variable position. Thus this design calculation provided an energy of substitution for each of the 20 amino acids at each variable position. FIG. 31a shows these results. At each variable position, the lowest energy substitution and all amino acid substitutions which are within 1 unit of energy of the lowest energy substitution are shown. Thus FIG. 31 a presents the most favorable substitutions for each of the variable positions. These results can be used to generate one or more experimental libraries which can be subsequently screened for improved antibody solubility. An experimental library was derived from this computational screening output by including the WT amino acid and all favorable polar amino acid substitutions at each variable position. As can be seen, no polar substitutions are predicted to be favorable for position 404, and so this position was left as the nonpolar WT phenylalanine in the library. This experimental library, which has a combinatorial complexity of 4.9×10⁸, is shown in FIG. 31b.

Affinity Maturation

As discussed above, a number of strategies can be applied for utilizing computational screening methodology to affinity mature antibodies.

### Example 14

rhumAb VEGF Affinity Maturation Using the Antibody/Antigen Complex Structure

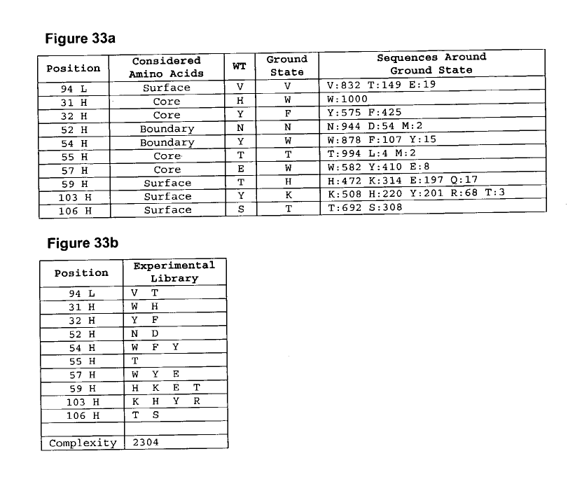

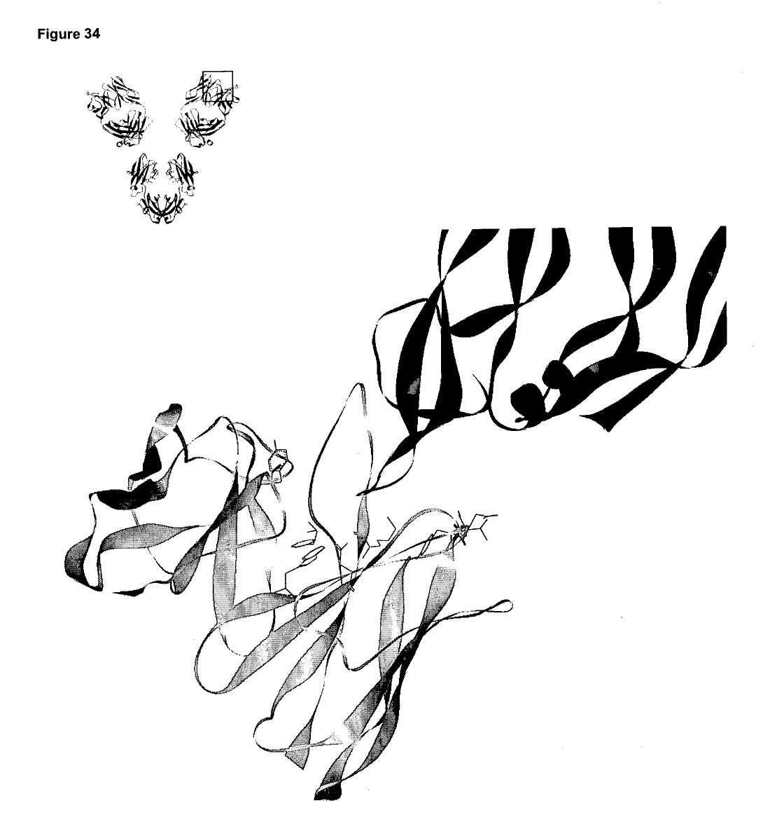

The availability of the bound antibody/antigen structure for rhumAb VEGF enables the affinity of this antibody to be enhanced directly using computational screening. More favorable interactions between the rhumAb VEGF antibody and its antigen were designed. Variable positions involved in mediating this interaction were chosen by visual inspection of the 1CZ8 structure, shown in FIG. 32 and listed in FIG. 33a. The set of amino acids allowed at variable positions was also chosen by visual inspection. Antigen residues which contact variable residue positions were floated. The conformations of amino acids at variable and floated positions were represented as a set of side chain rotamers derived from a backbone-independent rotamer library.

The 1CZ8 structure was used as the template for design calculations. The energies of all possible combinations of the considered amino acids at the chosen variable positions were calculated using a force field containing terms describing van der Waals, salvation, electrostatic, and hydrogen bond interactions, and the optimal (ground state) sequence was determined using a DEE algorithm. This ground state, and the WT rhumAb VEGF sequence, are shown in FIG. 33a. A diversity of sequences for an experimental library was generated by using a Monte Carlo algorithm to evaluate the energies of 1000 similar sequences around the predicted ground state. FIG. 33a shows the output sequence list from this Monte Carlo search.

These results can be used to generate one or more experimental libraries which can be screened for enhanced affinity for antigen. An experimental library, shown in FIG. 33b, was derived by applying a 5% cutoff of occupancy to the Monte Carlo output from each set of calculations, i.e. only amino acid substitutions which occur in 50 or greater variants out of the 1000 Monte Carlo output sequences are included in the library. Additionally, the WT amino acids at heavy chain positions 31, 54, 57, and 59 were added to the library so that the WT sequence is represented combinatorially in the library. This experimental library has a complexity of 2304.

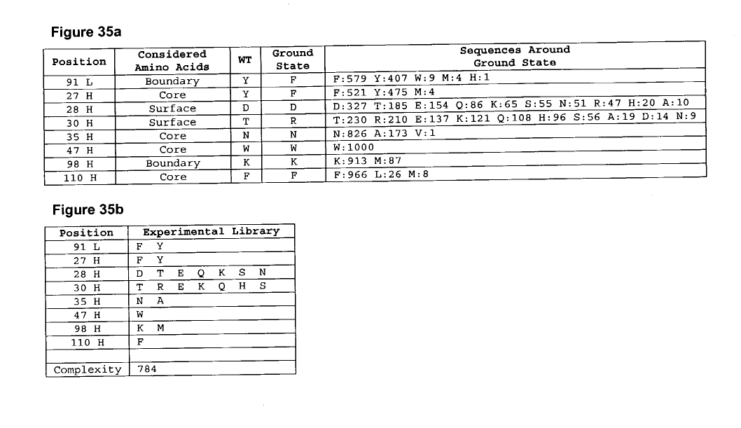

In another set of calculations, rhumAb VEGF was affinity matured by reengineering antibody residues which do not contact antigen. Here the variable positions in the design calculation were those residues which interact with contact residues, but are not themselves contact residues. As discussed above, by using computational screening to explore substitutions in the shell of residues which interact with contact residues, a quality diversity of new contact residue conformations can be sampled. Variable positions involved were chosen by visual inspection of the 1CZ8 structure, shown in FIG. 34 and listed in FIG. 35a. The set of amino acids allowed at variable positions was also chosen by visual inspection. The conformations of amino acids at variable positions were represented as a set of side chain rotamers derived from a backbone-independent rotamer library. The 1CZ8 structure was used as the template for design calculations. The energies of all possible combinations of the considered amino acids at the chosen variable positions were calculated using a force field containing terms describing van der Waals, salvation, electrostatic, and hydrogen bond interactions, and the optimal (ground state) sequence was determined using a DEE algorithm. This ground state, and the WT rhumAb VEGF sequence, are shown in FIG. 35a. A diversity of sequences for an experimental library was generated by using a Monte Carlo algorithm to evaluate the energies of 1000 similar sequences around the predicted ground state. FIG. 35a shows the output sequence list from this Monte Carlo search.

These results can be used to generate one or more experimental libraries which can be screened for enhanced affinity for antigen. An experimental library, shown in FIG. 35b, was derived by applying a 5% cutoff of occupancy to the Monte Carlo output from each set of calculations, i.e. only amino acid substitutions which occur in 50 or greater variants out of the 1000 Monte Carlo output sequences are included in the library. The WT is already represented in this library, and so no additional amino acids were added. This experimental library has a complexity of 784.

### Example 15

SM3 Affinity Maturation Using the Antibody/Antigen Complex Structure

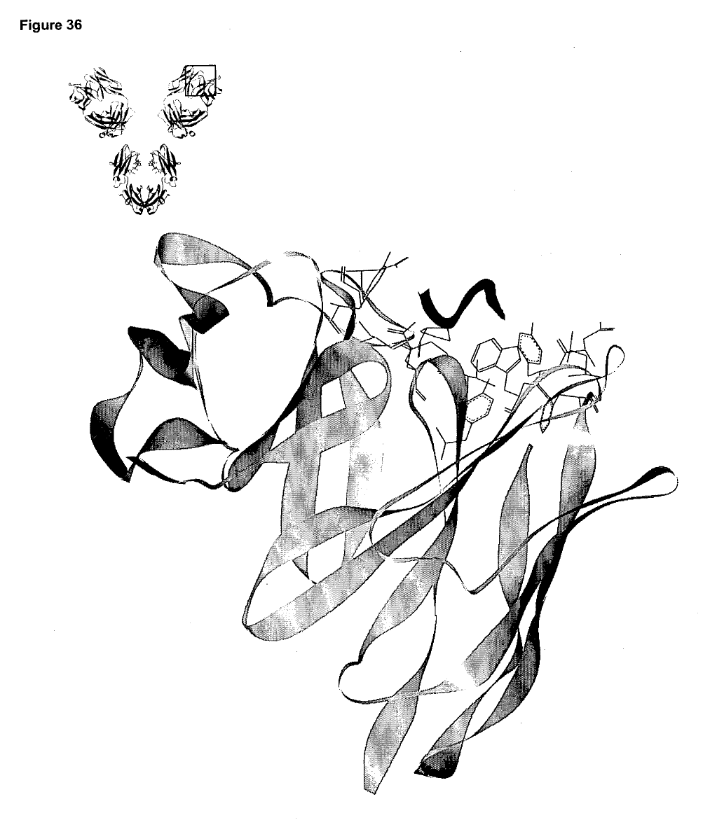

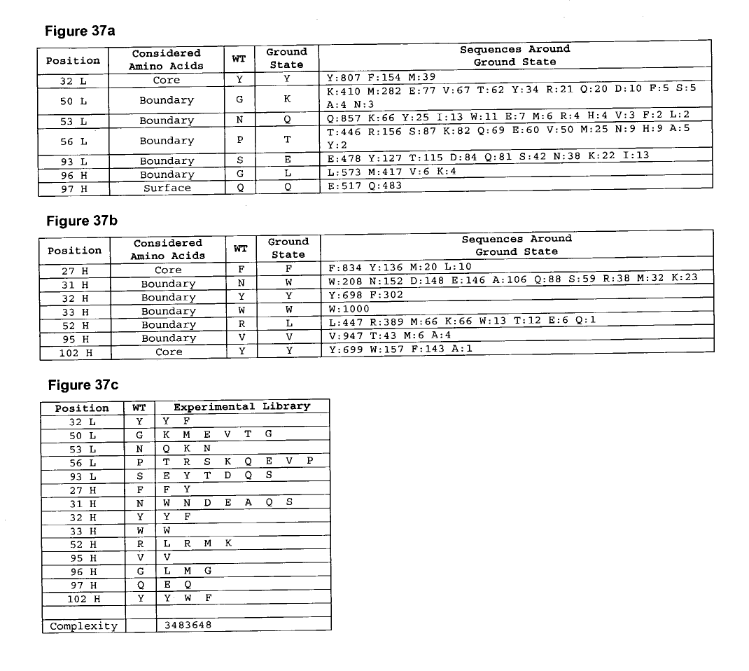

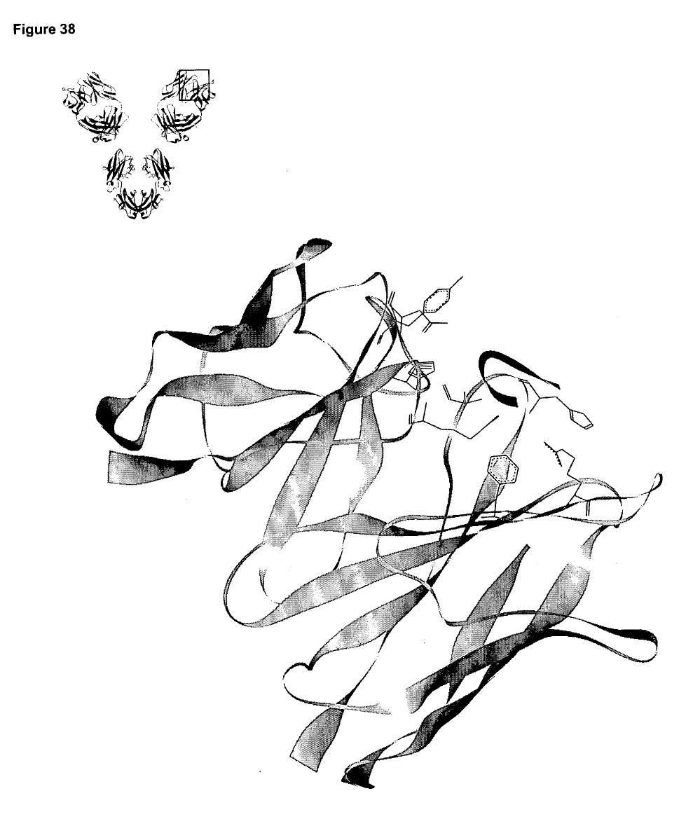

The availability of the bound antibody/antigen complex structure for SM3 enables the affinity of this antibody to be enhanced directly using computational screening. SM3 is a mouse antibody that is currently being developed as an anticancer agent. The high resolution structure is available of the complex of the SM3 Fab with its target antigen, a peptide from the cell surface mucin MUC1. This structure, PDB accession code 1SM3, served as the template for design calculations. More favorable interactions between the SM3 antibody and its antigen were designed. SM3 binds the MUC1 peptide using an extensive binding pocket which involves a large number or SM3 residues. The pocket can, however, be separated into two somewhat independent sets of residues, and thus in order to reduce the complexity of the computational screen, two separate sets of design calculations were carried out. Variable positions involved in mediating this interaction were chosen by visual inspection of the 1SM3 structure, shown in FIG. 36 and listed in FIGS. 37a and 37b. The set of amino acids allowed at variable positions was also chosen by visual inspection. Antigen residues were kept fixed in the two calculations. The conformations of amino acids at variable positions were represented as a set of side chain rotamers derived from a backbone-independent rotamer library.

The 1SM3 structure was used as the template for design calculations. For both sets of calculations, the energies of all possible combinations of the considered amino acids at the chosen variable positions were calculated using a force field containing terms describing van der Waals, salvation, electrostatic, and hydrogen bond interactions, and the optimal (ground state) sequences were determined using a DEE algorithm. These ground states, and the WT SM3 sequence, are shown in FIGS. 37a and 37b. A diversity of sequences for an experimental library was generated by using a Monte Carlo algorithm to evaluate the energies of 1000 similar sequences around the predicted ground states. FIGS. 37a and 37b show the output sequence lists from these Monte Carlo searches. These results can be used to generate one or more experimental libraries which can be subsequently screened for enhanced affinity for antigen. An experimental library, shown in FIG. 37c, was derived by applying a 5% cutoff of occupancy to the Monte Carlo output from each set of calculations, and then these primary libraries were subsequently combined to generate a secondary library with mutations at all positions. Additionally, the WT amino acids at light chain positions 50, 53, 56, and 93, and heavy chain position 96 were added to the library so that the WT sequence is represented combinatorially in the library. This may be particularly important here because some glycine and proline residues in the WT sequence were allowed to be variable in the calculations. These amino acids can be important determinants of protein backbone conformation, and therefore the benefit of their replacement with side chains which are capable of making favorable interaction with antigen may be outweighed by unfavorable potential backbone movements. This combinatorial library has a complexity of 3.5×10⁶.

### Example 16

Campath Affinity Maturation Using the Antibody/Antigen Complex Structure

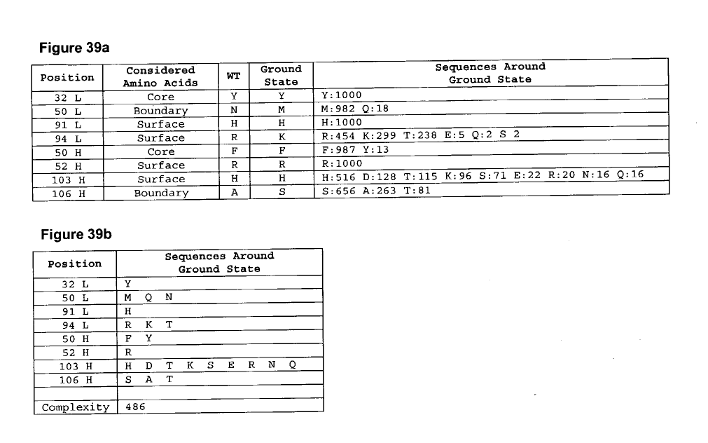

The availability of the bound antibody/antigen complex structure for Campath enables the affinity of this antibody to be enhanced directly using computational screening. More favorable interactions between the Campath antibody and its antigen were designed. Variable positions involved in mediating this interaction were chosen by visual inspection of the 1CE1 structure, shown in FIG. 38 and listed in FIG. 39a. The set of amino acids allowed at variable positions was also chosen subjectively by visual inspection. Antigen residues were floated. The conformations of amino acids at variable and floated positions were represented as a set of side chain rotamers derived from a backbone-independent rotamer library.

The 1CE1 structure was used as the template for design calculations. The energies of all possible combinations of the considered amino acids at the chosen variable positions were calculated using a force field containing terms describing van der Waals, solvation, electrostatic, and hydrogen bond interactions, and the optimal (ground state) sequence was determined using a DEE algorithm. This ground state and the WT Campath sequence are shown in FIG. 39a. A diversity of sequences for an experimental library was generated by using a Monte Carlo algorithm to evaluate the energies of 1000 similar sequences around the predicted ground state. FIG. 39a shows the output sequence list from this Monte Carlo search.

These results can be used to generate one or more experimental libraries which can be screened for enhanced affinity for antigen. An experimental library, shown in FIG. 39b, was derived by applying a 5% cutoff of occupancy to the Monte Carlo output from each set of calculations, i.e. only amino acid substitutions which occur in 50 or greater variants out of the 1000 Monte Carlo output sequences are included in the library. Additionally, the WT asparagine at light chain position 50 was added to the library so that the WT sequence is represented combinatorially in the library. This combinatorial library has a complexity of 486.

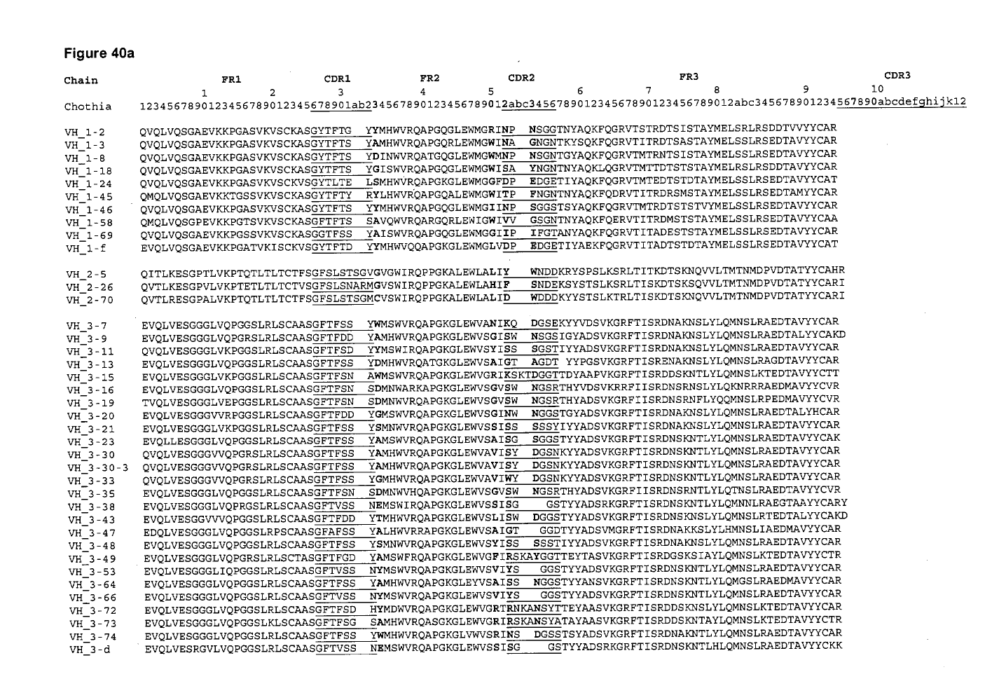

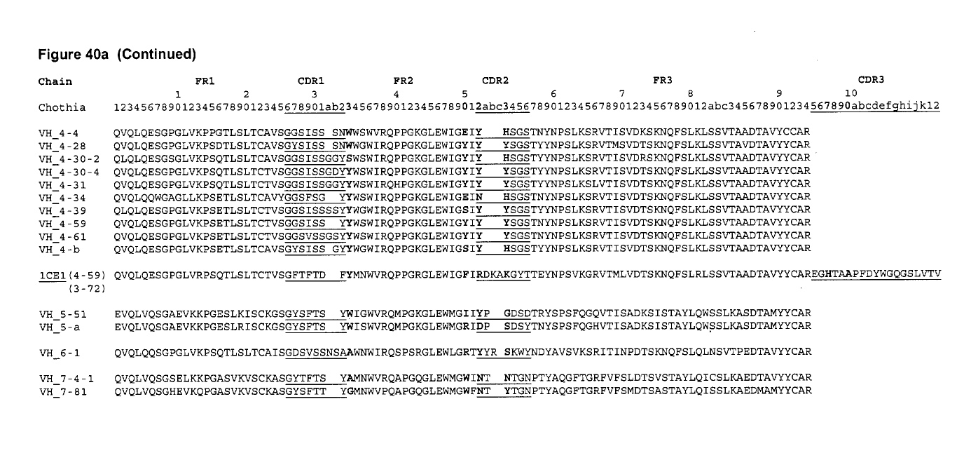

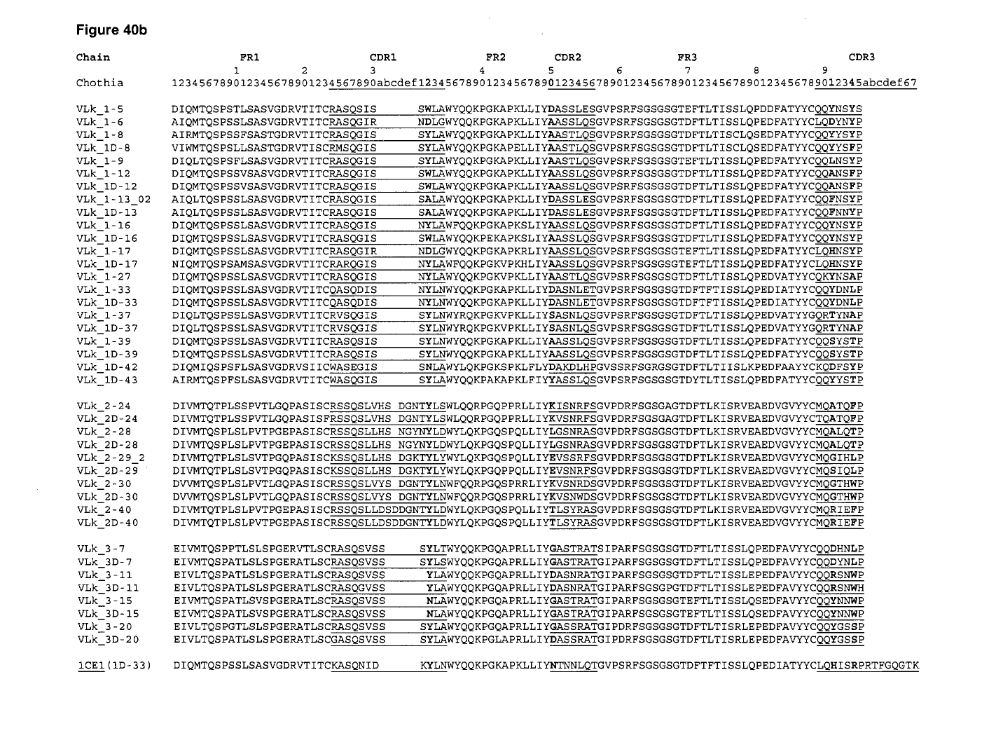

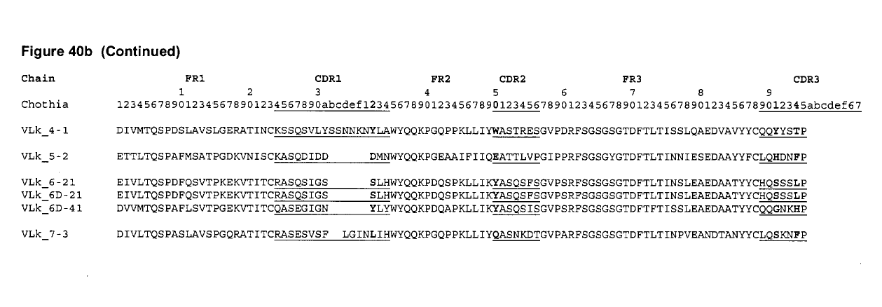

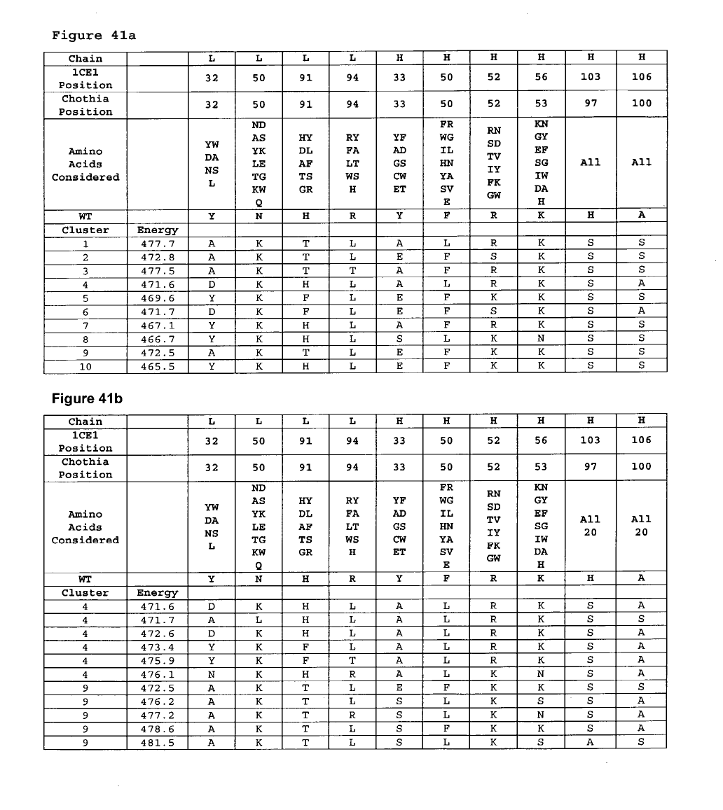

Because of the number of residues involved in mediating the interaction of Campath with its antigen, it may be beneficial to reduce the complexity of the design calculations. The use of sequence information here will enable the complexity of the computational problem to be reduced while ensuring that the remaining diversity sampled is of high quality, in terms of the structural, functional, and immunogenic fidelity of the antibody. Sequence information was used to guide the choice of variable positions and the set of amino acids considered at those positions for the Campath affinity maturation calculations. FIGS. 40a and 40b show the Campath heavy and light chain variable chain sequences aligned with the human V_{H} and V_{L} kappa germ line sequences. A new design calculation using this information was run to affinity mature Campath. The sequence information was first used to reevaluate the list of variable positions. A subset of the positions in FIG. 39a was chosen based on the degree of variability at each position in the germ line. The sequence information was used to choose the set of amino acids considered at variable positions in the new design calculation. All amino acids, and only those amino acids, which appear at each variable position in the germ line were considered in the new design calculation. For variable positions in CDR3, for which no sequence information is available, all 20 amino acids were considered. This set of amino acids is shown in FIG. 41a. Antigen residues were allowed to float during the calculations.

The 1CE1 structure was used as the template for design calculations. In this new calculation, energies of all possible combinations were not precalculated. Instead, a genetic algorithm was used to screen for low energy sequences, with energies being calculated during each round of “evolution” only for those sequences being sampled. The conformations of amino acids at variable and floated positions were represented as a set of side chain rotamers derived from a backbone-independent rotamer library using a flexible rotamer model. Energies were calculated using a force field containing terms describing van der Waals, salvation, electrostatic, and hydrogen bond interactions. This calculation generated a list of 300 sequences which are predicted to be low in energy. Clustering was performed to facilitate analysis of the results and library generation. The 300 output sequences were clustered computationally into 10 groups of similar sequences using a nearest neighbor single linkage hierarchical clustering algorithm to assign sequences to related groups based on similarity scores (Diamond, R., Coordinate-Based Cluster Analysis, Acta Cryst. 1995, D51, 127-135.). The 300 output sequences were clustered computationally into 10 groups of similar sequences. That is, all sequences within a group are most similar to all other sequences within the same group and less similar to sequences in other groups. The lowest energy sequence from each of these ten clusters, used here as a representative of each group, is presented in FIG. 41a.

These results can be used to generate one or more experimental libraries which can be subsequently screened for increased affinity for antigen. An experimental library can be derived directly from the representative cluster group sequences. Thus FIG. 41a provides a 10 sequence experimental library. To efficiently use experimental resources, this library size of 10 variants could be screened first, followed by subsequent screening of sequences or a subset of sequences within the group to which the experimentally determined most favorable variant belongs. For example, if variants 4 and 9 (i.e. the lowest energy sequences from cluster groups 4 and 9) were found experimentally to be most favorable, all of the sequences of cluster groups 4 and 9 could be subsequently screened. The 6 sequences in group 4 and 5 sequences in group 9 are presented in FIG. 41b as an example of such an experimental library.

### Example 17

D3H44 Affinity Maturation Using Complex and Uncomplexed Structures

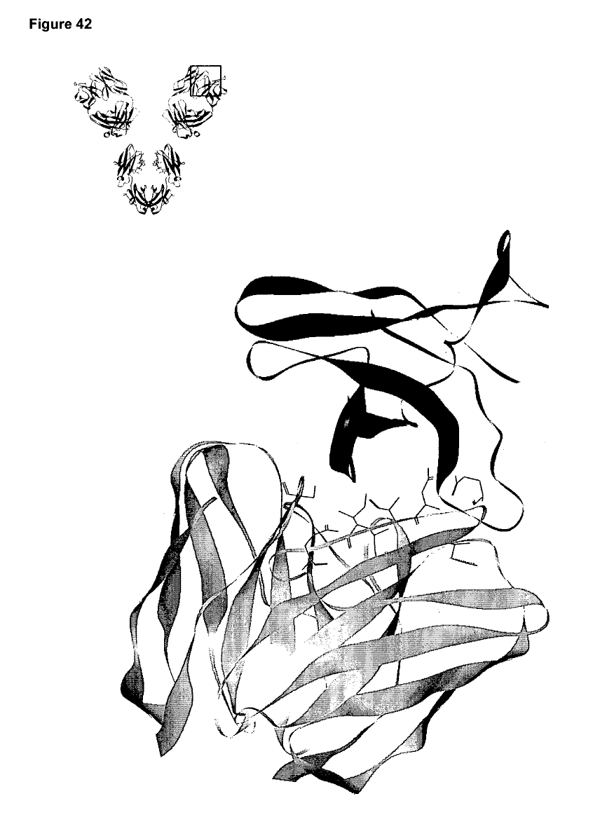

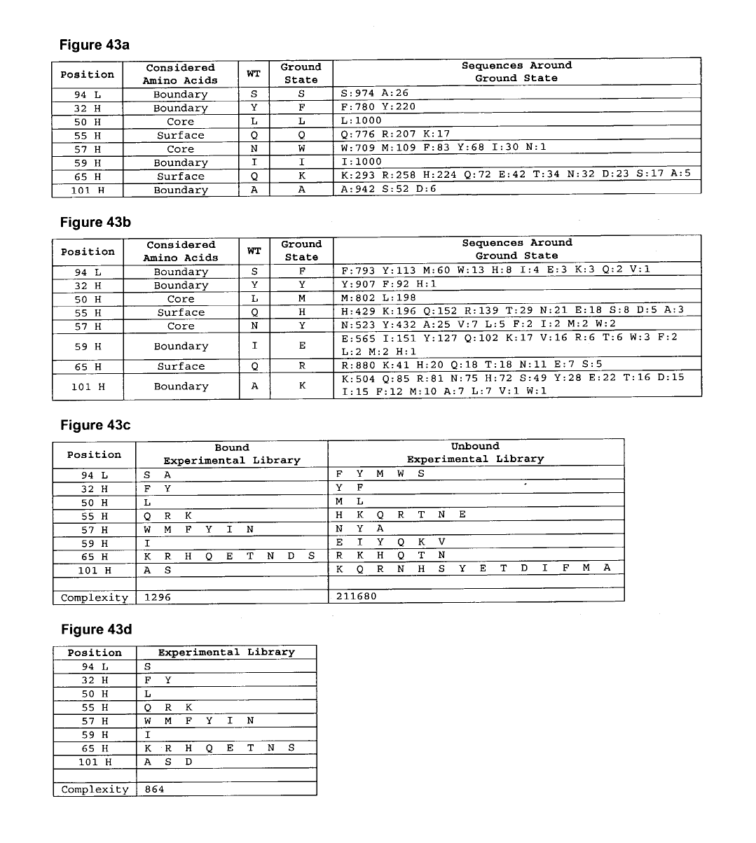

The availability of structural information for both the bound and unbound forms of the anti-tissue factor antibody D3H44 provide the opportunity to explore how both complexed and uncomplexed structural information can be used to computationally affinity mature an antibody. D3H44 is a humanized antibody that is currently being developed for treatment of thrombotic disorders. The high resolution structure of the D3H44 antibody/antigen complex, PDB accession code 1JPT, and the unbound antibody structure, PDB accession code 1JPS, served as templates in separate sets of design calculations aimed at designing more favorable interactions between the D3H44 antibody and its antigen. Variable positions involved in mediating this interaction were chosen by visual inspection of the 1JPT structure, shown in FIG. 42 and listed in FIG. 43a. The set of amino acids considered at variable positions was also chosen by visual inspection. Antigen residues which contact antibody variable position residues were floated in the bound structure calculation. The conformations of amino acids at variable and floated positions were represented as a set of side chain rotamers derived from a backbone-independent rotamer library.

The 1JPT and 1JPS structures were used as templates in two separate sets of design calculations. For both sets of calculations, the energies of all possible combinations of the considered amino acids at the chosen variable positions were calculated using a force field containing terms describing van der Waals, salvation, electrostatic, and hydrogen bond interactions, and the optimal (ground state) sequences were determined using a DEE algorithm. These ground states, and the WT D3H44 sequence, are shown in FIGS. 43a and 43b. A diversity of sequences for an experimental library was generated by using a Monte Carlo algorithm to evaluate the energies of 1000 similar sequences around the predicted ground states. FIGS. 43a and 43b show the output sequence lists from these Monte Carlo searches.

Notably, the diversity of sequences in the bound output is approximately a subset of the sequences in the unbound output. This result validates the use of using unbound structural information for affinity maturation, because it indicates that such calculations, while reducing sequence complexity for experimental screening, still produce quality antigen binding diversity. That is, experimental libraries derived from such calculations are enriched in sequences that favorably bind antigen. For example, experimental libraries were generated from the output of both bound and unbound calculations. These experimental libraries, shown in FIG. 43c, were derived by applying a 1% cutoff of occupancy to the Monte Carlo output from each set of calculations, i.e. only amino acid substitutions which occur in 10 or greater variants out of the 1000 Monte Carlo output sequences are included in the library. Additionally, WT amino acids were incorporated into the library if they were not already represented. The combinatorial complexities are 1296 and 211680 for the bound- and unbound-derived libraries respectively. As can be seen, a significant portion of the sequences present in the bound-derived library are present in the unbound-derived library, which is substantially reduced in complexity from random sequences.

The results from both sets of calculations can be combined to generate an experimental library. An experimental library, shown in FIG. 43d, was derived by including only those substitutions which are present in the Monte Carlo outputs of both bound and unbound design calculations. Additionally, the WT amino acid at light chain position 94 was added to the library so all of the WT amino acids are represented. This library provides a list of substitutions that are compatible with the antibody in both forms, ensuring that the derived library does not contain variants that are poorly behaved in the absence of antigen. Furthermore, substitutions which are favorable in the bound form but unfavorable in the unbound form may be due to the need for significant conformational changes for binding. Elimination of these substitutions may trim the library of unfavorable variants which lose entropy upon binding. This combinatorial library has a complexity of 864.

### Example 18

Herceptin Affinity Maturation Using the Uncomplexed Structure

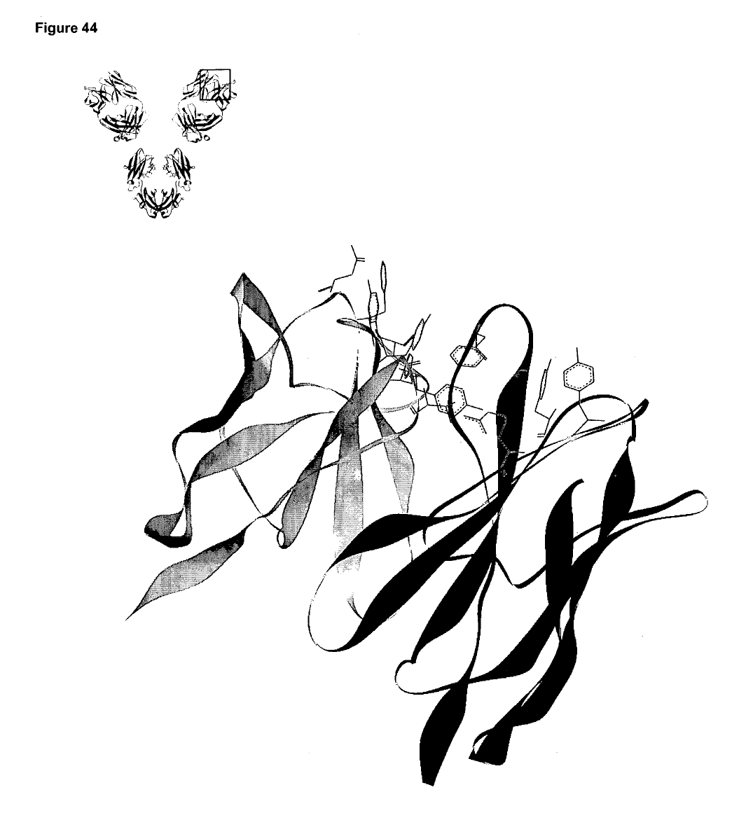

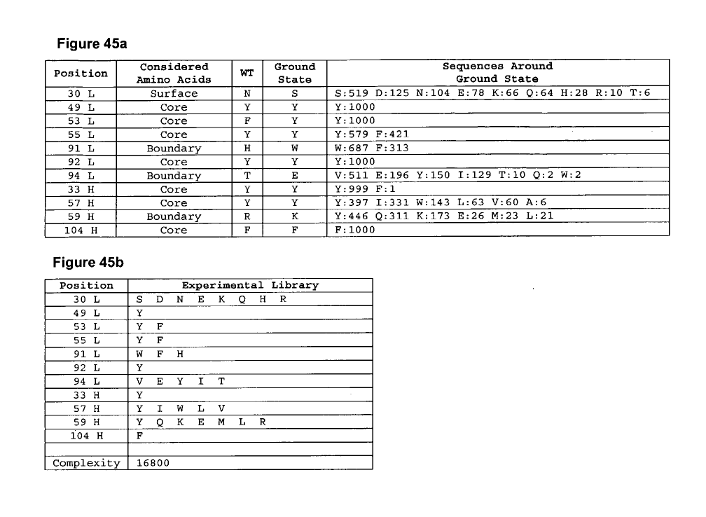

Although there is a structure available of the unbound Herceptin scFv antibody fragment, there is no available structure of the bound antibody/antigen complex. However, there is a wealth of experimental information available which can be used to guide affinity maturation design calculations. An alanine scanning mutagenesis study (Kelley et al., 1993, Biochemistry 32:6828-6835) showed that there are four central Herceptin residues, W, X, Y, and Z which are crucial for binding the Her2/neu antigen. A subsequent study used phage display to explore sequence diversity at these residues and residues proximal to them in the 1FVC structure (Gerstner et al., 2002, J. Mol. Biol. 321:851-862). The results from these studies were used to guide the choice of variable positions and amino acids considered at those positions in design calculations aimed at affinity maturing the Herceptin antibody. Here the goal is to utilize computational screening to generate a high quality library that is enriched for substitutions at antigen binding positions which are structurally compatible with the Herceptin antibody. Variable positions were chosen as those positions which show moderate variability in the phage display results. That is, positions that were very intolerant to mutation (one amino acid identity was observed in the majority of selected sequences), and positions that were very tolerant to mutation (no preference for amino acid identity was observed) were not chosen as variable positions. Mutations at these positions are expected to have a deleterious effect or no effect respectively on antigen binding. Positions that have some but not stringent amino acid requirements have the most value in terms of exploring diversity which may be more favorable for antigen binding. These positions are shown in FIG. 44 and listed in FIG. 45a. The set of amino acids considered at these variable positions was also guided by the experimental results. For a given position, if the diversity of substitutions observed was greater than 90% polar or nonpolar residues, the amino acids considered for that position were chosen as the set belonging to the surface or core classification respectively. If no trend was observed, the amino acids considered for that position were chosen as the set belonging to the boundary classification. The conformations of amino acids at variable positions were represented as a set of side chain rotamers derived from a backbone-independent rotamer library. The 1FVC structure was used as the template for design calculations. The energies of all possible combinations of the considered amino acids at the chosen variable positions were calculated using a force field containing terms describing van der Waals, solvation, electrostatic, and hydrogen bond interactions, and the optimal (ground state) sequence was determined using a DEE algorithm. This ground state, and the WT Herceptin sequence, are shown in FIG. 45a. A diversity of sequences for an experimental library was generated by using a Monte Carlo algorithm to evaluate the energies of 1000 similar sequences around the predicted ground state. FIG. 45a shows the output sequence list from this Monte Carlo search.

These results can be used to generate one or more experimental libraries which can be screened for enhanced affinity for antigen. An experimental library, shown in FIG. 45b, was derived by applying a 1% cutoff of occupancy to the Monte Carlo output from each set of calculations, i.e. only amino acid substitutions which occur in 10 or greater variants out of the 1000 Monte Carlo output sequences are included in the library. Additionally, the WT amino acids at light chain positions 53 and 91, and heavy chain positions 59 were added to the library so that the WT sequence is represented combinatorially in the library. This experimental library has a complexity of 16800.

All references cited herein are incorporated by reference in their entirety.

Whereas particular embodiments of the invention have been described above for purposes of illustration, it will be appreciated by those skilled in the art that numerous variations of the details may be made without departing from the invention as described in the appended claims.

We claim:

1. A method for optimizing at least one physico-chemical property of an antibody, said method executed by a computer under the control of a program, said computer including a memory for storing said program, said method comprising the steps of:
a. receiving a template antibody structure;
b. selecting at least one variable positions which belong to said template antibody structure;
c. selecting at least one amino acids to be considered at said variable positions;
d. analyzing the interaction of each of said amino acids at each variable position with at least part of the remainder of said antibody, including said amino acids at other variable positions; and
e. identifying a set of at least one antibody sequence with at least one optimized physico-chemical property.

2. A method according to claim 1, wherein at least one of the optimized physico-chemical properties is selected from the group consisting of stability, solubility, and antigen binding affinity.

3. A method according to claim 2, wherein at least one of the optimized physico-chemical properties is stability.

4. A method according to claim 3, wherein the stabilized portion of said antibody is selected from the group consisting of a domain and an interface between domains.

5. A method according to claim 4, wherein the stabilized portion of said antibody is a domain.

6. A method according to claim 4, wherein the stabilized portion of said antibody is an interface between domains.

7. A method according to claim 2, wherein the physico-chemical property is solubility.

8. A method according to claim 7, wherein at least one antibody sequence possesses an increase in polar character.

9. A method according to claim 7, wherein said selecting step further comprises selecting at least one nonpolar amino acid and substituting said nonpolar amino acid with a polar amino acid.

10. A method according to claim 7, wherein said selecting step further comprises altering the pI of the antibody.

11. A method according to claim 2, wherein at least one of the optimized physico-chemical properties is antigen binding affinity.

12. A method according to claim 11, wherein at least one of said variable positions is located in a framework region of the antibody.

13. A method according to claim 11, wherein at least one of said variable positions is located in a complementarity determining region (CDR) of the antibody.

14. A method according to claim 1, wherein each of said amino acids at each of said variable positions are represented as a group of potential rotamers.

15. A method according to claim 1, wherein at least two variable positions are selected and at least two amino acids are considered at each variable position.

16. A method according to claim 1, wherein said analyzing step further comprises a computational step utilizing at least two of the energy terms selected from the group consisting of van der Waals, electrostatics, hydrogen bonds and solvation.

17. A method according to claim 1, wherein said variable positions are chosen based on their level of variability in a set of aligned antibody sequences.

18. A method according to claim 1, wherein one said amino acids are chosen from a list of amino acids which occur at said position or positions in a set of aligned antibody sequences.

19. A method according to claim 1, wherein said analyzing step includes a Protein Design Automation program.

20. A method according to claim 1, wherein said analyzing step includes a Sequence Prediction Algorithm program.

21. A method according to claim 1, wherein said antibody is selected from the group consisting of a full-length antibody and an antibody fragment.

22. A method according to claim 1, wherein said antibody sequence is substantially encoded by at least one mammalian antibody gene.

23. A method according to claim 1, wherein said antibody is selected from the group consisting of a fully human antibody, a humanized antibody, a chimeric antibody, and an engineered antibody.

24. A method according to claim 1, further comprising f) generating a library from said set of at least one antibody sequence.

25. A method according to claim 24 wherein said library is a computational library.

26. A method according to claim 24 wherein said library is generated experimentally.

27. A method according to claim 24 further comprising g) experimentally screening said library.

28. A method according to claim 27, wherein said library is screened using at least one selection method.

29. A method according to claim 25, wherein said library is screened using at least one selection method selected from the group consisting of: phage display methods, cell surface display, in vitro display, and cytometric screening.

30. A method according to claim 25, wherein said selection method is a directed evolution method.

31. An antibody sequence from said library of claim 24.

32. An antibody sequence according to claim 28, wherein said antibody sequence is substantially encoded by a mammalian antibody gene.

33. An antibody identified from said screening of claim 24.

34. An antibody to claim according to claim 33, wherein said antibody is a full-length antibody or an antibody fragment.

35. An antibody according to claim 33, wherein said antibody is selected from the group consisting of a fully human antibody, a humanized antibody, a chimeric antibody, and an engineered antibody.

36. A method of treating a patient in need of said treatment, comprising administering an antibody of claim 28 to said patient.
